(19) United States
(12) Patent Application Publication
(10) Pub. No.: US 2025/0297332 A1
(43) Pub. Date: Sep. 25, 2025

(54) DEVICES, SYSTEMS, AND METHODS FOR CAPTURING TARGETS

(71) Applicant: The General Hospital Corporation, Boston, MA (US)

(72) Inventors: Shannon L. Stott, Winchester, MA (US); Daniel C. Rabe, Somerville, MA (US); Genevieve M. Boland, Somerville, MA (US); Seyed A. Rabi, Boston, MA (US)

(21) Appl. No.: 18/860,495

(22) PCT Filed: Apr. 26, 2023
(86) PCT No.: PCT/US2023/019973

§ 371 Date: Oct. 25, 2024

Related U.S. Application Data
(60) Provisional application No. 63/334,830, filed on Apr. 26, 2022.

(51) Int. Cl. C12Q 1/70; B01L 3/00; C07K 16/10; C07K 16/18; C12Q 1/6804; C12Q 1/6851

(52) U.S. Cl. CPC C12Q 1/701; B01L 3/502761; C07K 16/1003; C07K 16/18; C12Q 1/6804; C12Q 1/6851; B01L 2200/0647; B01L 2300/0819; B01L 2300/0877

(57) ABSTRACT

The present document relates to microfluidic devices and microfluidic systems for capturing a target of interest. Also described herein are methods of isolating or capturing such targets.

### CROSS-REFERENCE TO RELATED APPLICATION

This application claims the benefit of U.S. Provisional Patent Application No. 63/334,830, filed on Apr. 26, 2022, which is incorporated herein by reference in its entirety.

### STATEMENT OF GOVERNMENT INTEREST

This invention was made with Government support under Contract Nos. R01-CA226871 and F32-CA236417 awarded by National Cancer Institute and under Contract No. U18-TR003793 awarded by the National Center for Advancing Translational Sciences. The Government has certain rights in the invention.

### FIELD

The present document relates to devices, systems, and methods for capturing targets, e.g., target viruses, particles, vesicles, cells, and the like, from samples.

### BACKGROUND

Selective isolation of desired targets from a sample can simplify clinical diagnostics and basic medical research. Even still, recovery of targets in an intact form can be useful to understand their native structure, to elucidate clinically relevant metrics, and/or to perform analysis of purified, high quality test samples.

### SUMMARY

The present document relates to devices, systems, and methods for the selective capture and release of targets from a sample (e.g., a liquid sample). In some embodiments, a microfluidic device is employed to capture such targets. As described herein, arylene-containing linkers can be employed to provide a functionalized surface configured to capture such targets. Furthermore, the devices, systems, and methods herein can allow for capture of targets in an intact form. For instance, analysis of intact viruses or viral particles can provide additional information regarding infectivity, as compared to analysis of circulating viral RNA in a bulk sample.

Accordingly, in one aspect, the present document encompasses a microfluidic device comprising: a microchannel comprising an inner wall surface; a first linker covalently attached to the inner wall surface, or a portion thereof; a particle attached to the first linker; and a capture agent attached to the particle.

In some embodiments, the first linker comprises an arylene moiety. In some embodiments, the first linker comprises —Ar—NR^{N1}—, wherein Ar is optionally substituted arylene, and R^{N1} is hydrogen (H) or C₁₋₆ alkyl. In some embodiments, Ar is para-phenylene.

In some embodiments, the device further comprises a first binding pair disposed between first linker and the particle and/or a second binding pair disposed between the particle and the capture agent.

Any useful capture agent can be employed. In some embodiments, the capture agent is configured to interact with a surface of a virus in an intact form, a surface of a target cell, or a surface of a target vesicle (e.g., a target extracellular vesicle). In some embodiments, the capture agent comprises a molecule configured to bind a protein or a nucleic acid (e.g., DNA, RNA, or a modified form thereof). In some embodiments, the molecule comprises a protein or a nucleic acid. Other non-limiting examples of capture agents include angiotensin-converting enzyme 2 (ACE2), a mutant form thereof, or a recombinant form thereof; C—C chemokine receptor type 5 (CCR5), a mutant form thereof, or a recombinant form thereof; cluster of differentiation 4 (CD4), a mutant form thereof, or a recombinant form thereof; neutralizing antibody, KZ52, a mutant form thereof, or a recombinant form thereof; laminin-5, a mutant form thereof, or a recombinant form thereof; beparin sulfate proteoglycan, a mutant form thereof, or a recombinant form thereof; cluster of differentiation 46 (CD46), a mutant form thereof, or a recombinant form thereof; complement receptor type 2 (CR2), a mutant form thereof, or a recombinant form thereof, or a combination of any of these. Yet other examples of capture agents include an antibody, such as, e.g., an antibody that binds a spike protein of the virus or a receptor binding domain (RBD) of the virus; or an antibody that binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR); or an aptamer, such as, e.g., an aptamer that binds a spike protein of the virus. In some embodiments, the capture agent is configured to capture a virus (e.g., a cytomegalovirus, a coronavirus, an ebolavirus, an Epstein-Barr virus, a human immunodeficiency virus, an influenza virus, a hepatitis virus, or an oncovirus (e.g., a retrovirus, a herpesvirus, a papillomavirus, a polyomavirus, a hepatitis virus, and the like).

In some embodiments, the device further comprises; a second linker disposed between the particle and the capture agent. In some embodiments, the second linker comprise a flexible linker.

In some embodiments, the device further comprises: a binding pair between the particle and the flexible linker.

In another aspect, the present document encompasses a microfluidic system comprising: a first microchannel comprising a first inner wall surface, wherein at least one groove is defined in the first inner wall surface, and wherein the first microchannel comprises a first capture agent configured to interact with surface of a virus in an intact form.

In some embodiments, the system further comprises: a second microchannel comprising a second inner wall surface, wherein at least one groove is defined in the second inner wall surface, and wherein the second microchannel comprises a second capture agent configured to interact with a surface of a target cell or a surface of a target vesicle in an intact form.

In some embodiments, the system further comprises: a fluidic interconnect configured to provide fluidic communication between an outlet of the first microchannel to an inlet of the second microchannel.

In some embodiments of the system, the first microchannel is provided as the microchannel in the microfluidic device of any described herein. In some embodiments of the system, the second microchannel is provided as the microchannel in the microfluidic device of any described herein. In some embodiments, the first capture agent and the second capture agent are different.

In another aspect, the present document encompasses a microfluidic system comprising: a first microchannel comprising a first inner wall surface, wherein at least one groove is defined in the first inner wall surface, and wherein the first microchannel comprises a first capture agent configured to interact with a surface of a virus in an intact form; and a second microchannel comprising a second inner wall surface, wherein at least one groove is defined in the second inner wall surface, and wherein the second microchannel comprises a second capture agent configured to interact with a surface of a target cell or a surface of a target vesicle in an intact form.

In some embodiments, the system further comprises: a first fluidic interconnect configured to provide fluidic communication between an outlet of the first microchannel to an inlet of the second microchannel.

In some embodiments, the target cell comprises a B cell, an innate immune cell (e.g., a neutrophil, a macrophage, etc.), an epithelial cell, an endothelial cell, or a neural cell. In some embodiments, the target vesicle comprises a vesicle from a B cell, a vesicle from an innate immune cell, a vesicle from an epithelial cell, a vesicle from an endothelial cell, a vesicle from a neural cell, or a vesicle from a damaged cell.

In some embodiments, the system further comprises: a third microchannel comprising a third inner wall surface, wherein at least one groove is defined in the third inner wall surface, and wherein the third microchannel comprises a third capture agent configured to interact with a surface of a target cell or a surface of a target vesicle in an intact form. In some embodiments, the second capture agent and the third capture agent are different.

In some embodiments, the system further comprises: a second fluidic interconnect configured to provide fluidic communication between an outlet of the second microchannel to an inlet of the third microchannel.

In some embodiments of the system, the first microchannel is provided as the microchannel in the microfluidic device of any described herein. In some embodiments of the system, the second microchannel is provided as the microchannel in the microfluidic device of any described herein. In some embodiments of the system, the third microchannel is provided as the microchannel in the microfluidic device of any described herein.

In another aspect, the present document encompasses a method of isolating a virus in a sample, the method comprising: flowing the sample comprising the virus through a first microchannel comprising a first inner wall surface; capturing the virus in an intact form using a first capture agent in the first microchannel; lysing the intact form of the virus in the first microchannel, thereby providing a lysate; and analyzing the lysate to determine the presence of one or more markers of the virus.

In some embodiments, at least one groove is defined in the first inner wall surface of the first microchannel. In other embodiments, the first inner wall surface comprises the first capture agent configured to interact with a surface of the virus.

In some embodiments, said capturing comprises capturing at least one intact form of the virus in a microliter of the sample. In some embodiments, said capturing comprises contacting the virus in the intact form with the first capture agent. Any useful capture agent can be employed. In some embodiments, the first capture agent comprises a protein (e.g., angiotensin-converting enzyme 2 (ACE2), a mutant form thereof, or a recombinant form thereof; an antibody, such as an antibody that binds a spike protein of the virus or a receptor binding domain (RBD) of the virus; and the like) or an aptamer (e.g., an aptamer that binds a spike protein of the virus).

In some embodiments, said lysing comprises exposing the first microchannel to an elevated temperature, a lysing agent, or both.

In some embodiments, said analyzing comprises amplifying or sequencing the one or more markers. In particular embodiments, said amplifying comprises conducting an isothermal amplification reaction.

In some embodiments, the sample comprises a diluted sample, a stabilized sample, a preserved sample, or a combination thereof. In some embodiments, the sample comprises blood, plasma, stool, saliva, urine, sputum, or waste water.

In some embodiments, said flowing comprises flowing the sample through the microchannel of the microfluidic device of any described herein or through the first microchannel of the microfluidic system of any described herein.

In some embodiments, the method further comprises (e.g., prior to said flowing the sample): diluting the sample with a diluent to provide a diluted sample, wherein the diluted sample is used as the sample during said flowing of the sample through the first microchannel.

In some embodiments, the method further comprises (e.g., prior to said flowing the sample): stabilizing the sample with a stabilizer to provide a stabilized sample (e.g., by use of particular storage temperatures, platelet inhibitor cocktail, chemical additive, and the like). In some embodiments, the stabilized sample is used as the sample during said flowing of the sample through the first microchannel.

In some embodiments, the method further comprises (e.g., prior to said lysing the intact form of the virus): determining a concentration of the virus captured by the first capture agent in the first microchannel. In some embodiments, said determining comprises conducting one or more optical measurements, amplification reactions, sequencing, resistive pulse sensing, or particle analysis to measure a concentration of viral particles captured by the first capture agent in the first microchannel.

In some embodiments, the method further comprises (e.g., after said lysing the intact form of the virus): delivering one or more detection reagents to the first microchannel. In some embodiments, the one or more detection reagents are employed during said analyzing the lysate to conduct an isothermal amplification reaction within the first microchannel.

In some embodiments, the method further comprises (e.g., after said flowing the sample comprising virus though the first microchannel): collecting the sample after flowing through the first microchannel, thereby providing a collected sample comprising one or more target cells or target vesicles. In some embodiments, the method further comprises (e.g., after said collecting the sample after flowing through the first microchannel): flowing the collected sample through a second microchannel comprising a second inner wall surface. In some embodiments, at least one groove is defined in the second inner wall surface, wherein the second inner wall surface comprises a second capture agent configured to interact with a surface of the target cell or a surface of the target vesicle.

In some embodiments, the second capture agent comprises a protein (e.g., an antibody, optionally wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR)).

In yet another aspect, the present document encompasses a method of capturing targets in a sample, the method comprising: flowing the sample comprising a virus through a first microchannel comprising a first inner wall surface; capturing the virus in an intact form using a first capture agent in the first microchannel; and collecting the sample after flowing through the first microchannel, thereby providing a collected sample comprising one or more target cells or target vesicles. In some embodiments, the method further comprises (e.g., after said collecting the sample): flowing the collected sample through a second microchannel comprising a second inner wall surface; and capturing the target cell or the target vesicle in an intact form using the second capture agent in the second microchannel.

In some embodiments, at least one groove is defined in the first inner wall surface of the first microchannel. In some embodiments, the first inner wall surface comprises a first capture agent configured to interact with a surface of the virus.

In some embodiments, at least one groove is defined in the second inner wall surface of the second microchannel. In some embodiments, the second inner wall surface comprises a second capture agent configured to interact with a surface of the target cell or a surface of the target vesicle.

In some embodiments, the method further comprises: lysing the intact form of the virus in the first microchannel, thereby providing a first lysate; and analyzing the first lysate to determine the presence of one or more markers of the virus. In some embodiments, the method further comprises: lysing the target cell or the target vesicle in the second microchannel, thereby providing a second lysate; and analyzing the second lysate to determine the presence of one or more markers of the target cell or the target vesicle. In particular embodiments, said lysing comprises exposing the first microchannel and/or the second microchannel to an elevated temperature, a lysing agent, or both.

In some embodiments, said analyzing comprises amplifying or sequencing the one or more markers (e.g., comprising a nucleic acid). In some embodiments, said amplifying comprises conducting an isothermal amplification reaction.

In some embodiments, said flowing comprises flowing the sample through the microchannel of the microfluidic device of any described herein or through the first microchannel of the microfluidic system of any described herein.

In some embodiments, the method further comprises (e.g., prior to said flowing the sample): diluting the sample with a diluent to provide a diluted sample, wherein the diluted sample is used as the sample during said flowing of the sample through the first microchannel.

In some embodiments, the method further comprises (e.g., prior to said flowing the sample): stabilizing the sample with a stabilizer to provide a stabilized sample (e.g., by use of particular storage temperatures, platelet inhibitor cocktail, chemical additive, and the like). In some embodiments, the stabilized sample is used as the sample during said flowing of the sample through the first microchannel.

In some embodiments, the method further comprises (e.g., prior to said lysing the intact form of the virus): determining a concentration of the virus captured by the first capture agent in the first microchannel. In some embodiments, said determining comprises conducting one or more optical measurements, amplification reactions, sequencing, resistive pulse sensing, or particle analysis to measure a concentration of viral particles captured by the first capture agent in the first microchannel.

In some embodiments, the method further comprises (e.g., after said lysing the intact form of the virus): delivering one or more detection reagents to the first microchannel. In some embodiments, the one or more detection reagents are employed during said analyzing the lysate to conduct an isothermal amplification reaction within the first microchannel.

In some embodiments, the method further comprises (e.g., after said flowing the sample comprising virus though the first microchannel): collecting the sample after flowing through the first microchannel, thereby providing a collected sample comprising one or more target cells or target vesicles; and flowing the collected sample through a second microchannel comprising a second inner wall surface. In some embodiments, at least one groove is defined in the second inner wall surface, and the second inner wall surface comprises a second capture agent configured to interact with a surface of the target cell or a surface of the target vesicle.

In another aspect, the present document comprises a method of determining viral load in a sample, the method comprising: flowing the sample through a first microchannel comprising a first inner wall surface, wherein at least one groove is defined in the first inner wall surface, and wherein the first inner wall surface comprises a first capture agent configured to interact with a surface of a viral particle; capturing the viral particle in an intact form using the first capture agent in the first microchannel; and measuring a concentration of viral particle captured in the first microchannel.

In some embodiments, said measuring comprises conducting one or more optical measurements, amplification reactions, sequencing, resistive pulse sensing, or particle analysis to measure a concentration of viral particles captured by the first capture agent in the first microchannel.

In some embodiments, the method further comprises (e.g., after said capturing): lysing the viral particle in the first microchannel, thereby providing a lysate; and analyzing the lysate to determine the presence of one or more markers of the viral particle.

In some embodiments, the method further comprises (e.g., after said capturing): collecting the sample after flowing through the first microchannel, thereby providing a collected sample comprising one or more target cells or target vesicles; flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle; and capturing the target cell or the target vesicle in an intact form using the second capture agent in the second microchannel.

In some embodiments, the method further comprises: lysing the target cell or the target vesicle in the second microchannel, thereby providing a second lysate; and analyzing the second lysate to determine the presence of one or more markers of the target cell or the target vesicle. In some embodiments, said lysing comprises exposing the first microchannel and/or the second microchannel to an elevated temperature, a lysing agent, or both.

In some embodiments, said analyzing comprises amplifying or sequencing the one or more markers (e.g., a nucleic acid). In some embodiments, said amplifying comprises conducting an isothermal amplification reaction.

In some embodiments, In said flowing comprises flowing the sample through the microchannel of the microfluidic device of any described herein or through the first microchannel of the microfluidic system of any described herein.

In some embodiments, the method further comprises (e.g., prior to said flowing the sample): diluting the sample with a diluent to provide a diluted sample, wherein the diluted sample is used as the sample during said flowing of the sample through the first microchannel.

In some embodiments, the method further comprises (e.g., prior to said flowing the sample): stabilizing the sample with a stabilizer to provide a stabilized sample (e.g., by use of particular storage temperatures, platelet inhibitor cocktail, chemical additive, and the like), wherein the stabilized sample is used as the sample during said flowing of the sample through the first microchannel.

In some embodiments, the method further comprises (e.g., prior to said lysing the intact form of the virus): determining a concentration of the virus captured by the first capture agent in the first microchannel. In some embodiments, said determining comprises conducting one or more optical measurements, amplification reactions, sequencing, resistive pulse sensing, or particle analysis to measure a concentration of viral particles captured by the first capture agent in the first microchannel.

In some embodiments, the method further comprises (e.g., after said lysing the intact form of the virus): delivering one or more detection reagents to the first microchannel. In some embodiments, the one or more detection reagents are employed during said analyzing the lysate to conduct an isothermal amplification reaction within the first microchannel.

In some embodiments, the method further comprises (e.g., after said flowing the sample comprising virus though the first microchannel): collecting the sample after flowing through the first microchannel, thereby providing a collected sample comprising one or more target cells or target vesicles; and flowing the collected sample through a second microchannel comprising a second inner wall surface. In some embodiments, at least one groove is defined in the second inner wall surface, and the second inner wall surface comprises a second capture agent configured to interact with a surface of the target cell or a surface of the target vesicle.

In some embodiments, the second capture agent comprises a protein (e.g., an antibody, and optionally wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR)).

In another aspect, the present document encompasses a method of preparing a microfluidic device, the method comprising: forming an aryl-onium salt comprising an arylene moiety disposed between an onium group and a nucleophilic group; releasing the onium group to form an aryl radical; and exposing the aryl radical to a surface of a microfluidic device. In some embodiments, the method thereby provides a functionalized surface.

In some embodiments, the method further comprises (e.g., before said releasing): reacting the nucleophilic group of the aryl-onium salt with a first member of a first binding pair to form an aryl conjugate. In some embodiments, the aryl conjugate comprises the arylene moiety disposed between the onium group and the first member of the binding pair. In some embodiments, the aryl conjugate is employed during said releasing to provide the aryl radical.

In some embodiments, the method further comprises (e.g., after said exposing): reacting the nucleophilic group of the functionalized surface with a first member of a first binding pair.

In some embodiments, the method further comprises: providing a particle comprising a second member of the first binding pair, wherein the first and second members bind together to form a bond. In some embodiments, the method further comprises: attaching one or more capture agents to the particle.

In some embodiments, a linker (e.g., any described herein) is disposed between at least one of the one or more capture agents and the particle. In some embodiments, the linker comprises a flexible linker.

In some embodiments, a second binding pair is disposed between the particle and the flexible linker. In some embodiments, the second member of the first binding pair of the particle is employed as a first member of the second binding pair. In some embodiments, a second member of the second binding pair is attached to the flexible linker.

In some embodiments, the method further comprises: providing a linker comprising a second member of the first binding pair, wherein the first and second members bind together to form a bond.

In some embodiments, the method further comprises: attaching one or more capture agents to the linker (e.g., a flexible linker). In some embodiments, a second binding pair is disposed between the flexible linker and at least one of the one or more capture agents.

In some embodiments, said forming comprises exposing an arylene compound to an oxidant (e.g., nitrous acid or a nitrite salt) and an optional acid (e.g., hydrogen halide).

In some embodiments, the onium group and the nucleophilic group are in a para position. In some embodiments, the arylene moiety comprises phenylene; or wherein the onium group comprises diazonium, iodonium, bromonium, or sulfonium. In some embodiments, the nucleophilic group comprises amino (e.g., —NR^{N1}R^{N2}, wherein each of R^{N1} and R^{N2} is, independently, hydrogen or C₁₋₆ alkyl). In some embodiments, a terminal amino group (e.g., of the aryl-onium salt, the arylene moiety, the nucleophilic group, the aryl radical, the aryl conjugate, the particle, or the linker) is reacted prior to addition to the microfluidic device or exposure to the surface of the microfluidic device.

In some embodiments, said releasing comprises exposing the onium group to radiation (e.g., ultraviolet radiation), heat, or electric field. In particular embodiments, said releasing the onium group occurs in the presence of the surface of the microfluidic device.

In some embodiments, the surface of the microfluidic device comprises an inner wall surface of a microchannel. In particular embodiments, at least one groove is defined in the inner wall surface. In some embodiments, the inner wall surface comprises a plurality of grooves arranged and configured to generate chaotic flow within a fluid sample traveling through the microchannel.

In any embodiment herein (e.g., in a device, a system, or a method), the microchannel comprises at least one groove is defined in the inner wall surface of the microchannel (e.g., a first, second, third, or other microchannel). In some embodiments, the inner wall surface comprises a plurality of grooves arranged and configured to generate chaotic flow within a fluid sample traveling through the microchannel (e.g., a first, second, third, or other microchannel).

In any embodiment herein (e.g., in a device, a system, or a method), the target is a virus. In some embodiments, the virus is in an intact form. In other embodiments, the virus is a viral particle (e.g., a virus in an intact form that is a viral particle). Non-limiting examples of viruses include a cytomegalovirus, a coronavirus, an ebolavirus, an Epstein-Barr virus, a human immunodeficiency virus, an influenza virus, a hepatitis virus, or an oncovirus (e.g., a retrovirus, a herpesvirus, a papillomavirus, a polyomavirus, a hepatitis virus, and the like), as well as others described herein.

In any embodiment herein (e.g., in a device, a system, or a method), the target cell is an immune cell (e.g., a T cell, a B cell, or an innate immune cell), an epithelial cell, an endothelial cell, or a neural cell.

In any embodiment herein (e.g., in a device, a system, or a method), the target vesicle is an extracellular vesicle, a vesicle from an immune cell (e.g., a T cell, a B cell, or an innate immune cell), a vesicle from an epithelial cell, a vesicle from an endothelial cell, a vesicle from a neural cell, or a vesicle from a damaged cell.

In any embodiment herein (e.g., in a device, a system, or a method), the capture agent (e.g., a first, second, third, or other capture agent) comprises a protein (e.g., an antibody) or a nucleic acid (e.g., an aptamer). In some embodiments, the protein comprises angiotensin-converting enzyme 2 (ACE2), a mutant form thereof, or a recombinant form thereof. Other non-limiting examples of capture agents include C—C chemokine receptor type 5 (CCR5), a mutant form thereof, or a recombinant form thereof; cluster of differentiation 4 (CD4), a mutant form thereof, or a recombinant form thereof; neutralizing antibody, KZ52, a mutant form thereof, or a recombinant form thereof; laminin-S, a mutant form thereof, or a recombinant form thereof; heparin sulfate proteoglycan, a mutant form thereof, or a recombinant form thereof; cluster of differentiation 46 (CD46), a mutant form thereof, or a recombinant form thereof; complement receptor type 2 (CR2), a mutant form thereof, or a recombinant form thereof, or a combination of any of these. Yet other examples of capture agents include an antibody, such as, e.g., an antibody that binds a spike protein of the virus or a receptor binding domain (RBD) of the virus; or an antibody that binds to CD3, CD4, CD8, CD9, CD11b, CD14, CD16, CD19, CD20, CD31, CD45, CD63, CD66, CD66b, CD81, HLA-DR, TSG-101, epithelial cell adhesion molecule (EpCAM), epidermal growth factor receptor (EGFR), human epidermal growth factor receptor 2 (HER2), podoplanin, barrier-to-autointegration factor (BAF), platelet-derived growth factor receptor (PDGF), or ephrin receptor A2 (EphA2); an aptamer, such as, e.g., an aptamer that binds a spike protein of the virus; lectin; heparin; a glycoprotein; a deoxyribonucleic (DNA) fragment, and the like. Yet other capture agents are described herein.

In any embodiment herein (e.g., in a device, a system, or a method), one or more markers may be present (e.g., indicative of the target). In some embodiments, one or more markers may be analyzed. In some embodiments, the one or more markers comprises a nucleic acid.

In any embodiment herein (e.g., in a device, a system, or a method), a sample may be employed to provide the target. In some embodiments, the sample comprises a diluted sample, a stabilized sample, a preserved sample, or a combination thereof. In some embodiments, the sample comprises blood, plasma, stool, saliva, urine, sputum, or waste water.

### Definitions

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2(4):482-9) as incorporated into GeneMatcher Plus™ (Schwarz and Dayhof, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to an uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis., 53705). Such software may match similar sequences by assigning degrees of homology to various substitutions, deletions, and other modifications.

By “aliphatic” is meant a hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl, e.g., as described herein), alkenes (or alkenyl), alkynes (or alkynyl), including cyclic versions thereof, and further including straight- and branched-chain arrangements, and all stereo and position isomers as well. Such a hydrocarbon can be unsubstituted or substituted with one or more groups, such as groups described herein for an alkyl group.

By “alkyl” and the prefix “alk” is meant a branched or unbranched saturated hydrocarbon group of 1 to 24 carbon atoms, such as methyl (Me), ethyl (Et), n-propyl (n-Pr), isopropyl (i-Pr), cyclopropyl, n-butyl (n-Bu), isobutyl (i-Bu), s-butyl (s-Bu), t-butyl (t-Bu), cyclobutyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one or more alkenyl, alkoxy (e.g., —OR, in which R is optionally substituted aliphatic or optionally substituted alkyl), alkynyl, amino (e.g., —NR^{N1}R^{N2}, in which each of R^{N1} and R^{N2} is, independently, H, optionally substituted aliphatic, optionally substituted alkyl, optionally substituted aromatic, or optionally substituted aryl; or in which R^{N1} and R^{N2}, taken together, form an optionally substituted alkylene or heteroalkylene (e.g., as described herein)), aryl, carboxyaldehyde (e.g., —C(O) H), carboxyl (e.g., —CO₂H), cyano (e.g., —CN), halo, hydroxyl (e.g., —OH), nitro (e.g., —NO₂), oxo (e.g., ═O), and the like. The alkyl group can be a primary, secondary, or tertiary alkyl group substituted with one or more substituents (e.g., one or more halo or alkoxy). In some embodiments, the unsubstituted alkyl group is a C₁₋₃, C₁₋₄, C₁₋₆, C₁₋₈, C₁₋₁₀, C₁₋₁₂, C₁₋₁₆, C₁₋₁₈, C₁₋₂₀, C₁₋₂₄, C₂₋₆, C₂₋₈, C₂₋₁₀, C₂₋₁₂, C₂₋₁₆, C₂₋₁₈, C₂₋₂₀, C₂₋₂₄, C₃₋₈, C₃₋₁₀, C₃₋₁₂, C₃₋₁₆, C₃₋₁₈, C₃₋₂₀, or C₃₋₂₄ alkyl group.

By “alkylene” is meant a multivalent (e.g. bivalent) form of an aliphatic group or an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “alkaryl” is meant an aromatic group or an aryl group, as defined herein, attached to the parent molecular group through an alkylene group, as defined herein. Similarly, by the term “alkheteroaryl” is meant a heteroaryl group, as defined herein, attached to the parent molecular group through an alkylene group. Other groups preceded by the prefix “alk-” are defined in the same manner. The alkaryl group can be substituted or unsubstituted. For example, the alkaryl group can be substituted with one or more substitution groups, as described herein for alkyl and/or aryl. Exemplary unsubstituted alkaryl groups are of from 7 to 16 carbons (C₇₋₁₆ alkaryl), as well as those having an alkylene group with 1 to 6 carbons and an aryl group with 4 to 18 carbons (i.e., C₁₋₆ alk-C₄₋₁₈ aryl).

By “alkoxy” is meant-OR, where R is an optionally substituted alkyl group, as described herein. Exemplary alkoxy groups include methoxy, ethoxy, butoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “amino” is meant a —NR^{N1}R^{N2} group, in which each of R^{N1} and R^{N2} is, independently, H, optionally substituted aliphatic, optionally substituted alkyl, optionally substituted aromatic, or optionally substituted aryl; or R^{N1} and R^{N2}, taken together with the nitrogen atom to which each are attached, form a heterocyclyl (e.g., as described herein); or R^{N1} and R^{N2}, taken together, form an optionally substituted alkylene or heteroalkylene (e.g., as described herein).

By “ammonium” is meant a group including a protonated nitrogen atom N⁺. Exemplary ammonium groups include —N⁺R^{N1}R^{N2}R^{N3} where each of R^{N1}, R^{N2}, and R^{N3} is, independently, H, optionally substituted aliphatic, optionally substituted alkyl, optionally substituted aromatic, optionally substituted aryl, or optionally substituted alkaryl; or R^{N1} and R^{N2}, taken together with the nitrogen atom to which each are attached, form a heterocyclyl (e.g., as described herein); or R^{N1} and R^{N2}, taken together, form an optionally substituted alkylene or heteroalkylene (e.g., as described herein).

By “aromatic” is meant a cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Huckel rule (4n+2). The point of attachment to the parent structure typically is through an aromatic portion of the condensed ring system.

By “aryl” is meant an aromatic carbocyclic group comprising at least five carbon atoms to 15 carbon atoms (C₅₋₁₅), such as five to ten carbon atoms (C₅₋₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as alkyl, as well as any substitution groups described herein for alkyl. Exemplary aryl groups include, but are not limited to, benzyl, naphthalene, phenyl, biphenyl, phenoxybenzene, and the like. The term aryl also includes heteroaryl, which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term non-heteroaryl, which is also included in the term aryl, defines a group that contains an aromatic group that does not contain a heteroatom. In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylene” is meant a multivalent (e.g., bivalent) form of an aromatic or aryl group, as described herein. Exemplary arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for alkyl or aryl. The arylene group can include one or more heteroatoms, which include, but are not limited to, nitrogen, oxygen, sulfur, and phosphorus.

By “diazonium” is meant a group including —N₂⁺.

By “halo” is meant F, Cl, Br, or I.

By “halonium” is meant a group including —X⁺, where X is halo as defined herein. Exemplary halonium groups include an iodonium group (e.g., —I⁺), a bromonium group (e.g., —Br⁺), a chloronium group (e.g., —Cl⁺), or a fluoronium group (e.g., —F⁺).

By “heterocyclyl” is meant a 5-, 6- or 7-membered ring, unless otherwise specified, containing one, two, three, or four non-carbon heteroatoms (e.g., independently selected from the group consisting of nitrogen, oxygen, phosphorous, sulfur, or halo). The 5-membered ling has zero to two double bonds and the 6- and 7-membered rings have zero to three double bonds. The term “heterocyclyl” also includes bicyclic, tricyclic, tetracyclic, and other multicyclic groups.

By “heteroaliphatic” is meant an aliphatic group, as defined herein, containing one, two, three, or four non-carbon heteroatoms (e.g., independently selected from the group consisting of nitrogen, oxygen, phosphorous, sulfur, or halo). Heteroaliphatic groups may be substituted with one or more groups other than hydrogen, such as alkyl, as well as any substitution groups described herein for alkyl.

By “heteroalkyl” is meant an alkyl group, as defined herein, containing one, two, three, or four non-carbon heteroatoms (e.g., independently selected from the group consisting of nitrogen, oxygen, phosphorous, sulfur, or halo). Heteroalkyl groups may be substituted with one or more groups other than hydrogen, such as alkyl, as well as any substitution groups described herein for alkyl.

By “heteroalkylene” is meant a multivalent (e.g., bivalent form) of an aliphatic group or an alkyl group, as defined herein, containing one, two, three, or four non-carbon heteroatoms (e.g., independently selected from the group consisting of nitrogen, oxygen, phosphorous, sulfur, or halo).

By “hydroxyl” is meant a —OH group.

By “oxonium” is meant a group including a protonated oxygen atom O⁺. Exemplary oxonium groups include —O⁺R^{O1}R^{O2}, where each of R^{O1} and R^{O2} is, independently, H, optionally substituted aliphatic, optionally substituted alkyl, optionally substituted aromatic, optionally substituted aryl, or optionally substituted alkaryl; or wherein Rol and R^{O2}, taken together, form an optionally substituted alkylene or heteroalkylene (e.g., as described herein).

By “phosphinyl” is meant a —PR^{P1}R^{P2} group, in which each of R^{P1} and R^{P2} is, independently, H, optionally substituted aliphatic, optionally substituted alkyl, optionally substituted aromatic, optionally substituted aryl, or optionally substituted alkaryl; or R^{P1} and R^{P2}, taken together, form an optionally substituted alkylene or heteroalkylene (e.g., as described herein).

By “phosphonium” is meant a group including a protonated phosphorous atom P⁺. Exemplary phosphonium groups include —P⁺R^{P1}R^{P2}R^{R3}, where each of R^{P1}, R^{P2}, and R^{P3} is, independently, H, optionally substituted aliphatic, optionally substituted alkyl, optionally substituted aromatic, optionally substituted aryl, or optionally substituted alkaryl; or wherein two of R^{P1}, R^{P2}, and R^{P3}, taken together, form an optionally substituted alkylene or heteroalkylene (e.g., as described herein).

By “protecting group” is meant any group intended to protect a reactive group against undesirable synthetic reactions. Commonly used protecting groups are disclosed in “Greene's Protective Groups in Organic Synthesis,” John Wiley & Sons, New York, 2007 (4th ed., eds. P. G. M. Wuts and T. W. Greene), which is incorporated herein by reference. O-protecting groups include an optionally substituted alkyl group (e.g., forming an ether with reactive group O), such as methyl, methoxymethyl, methylthiomethyl, benzoyloxymethyl, t-butoxymethyl, etc.; an optionally substituted alkanoyl group (e.g., forming an ester with the reactive group O), such as formyl, acetyl, chloroacetyl, fluoroacetyl (e.g., perfluoroacetyl), methoxyacetyl, pivaloyl, t-butylacetyl, phenoxyacetyl, etc.; an optionally substituted aryloyl group (e.g., forming an ester with the reactive group O), such as —C(O)—Ar, including benzoyl; an optionally substituted alkylsulfonyl group (e.g., forming an alkylsulfonate with reactive group O), such as —SO₂—R^{S1}, where R^{S1} is optionally substituted C₁₋₁₂ alkyl, such as mesyl or benzylsulfonyl, an optionally substituted arylsulfonyl group (e.g., forming an arylsulfonate with reactive group O), such as —SO₂—R^{S4}, where R^{S4} is optionally substituted C₄₋₁₈ aryl, such as tosyl or phenylsulfonyl; an optionally substituted alkoxycarbonyl or aryl oxycarbonyl group (e.g., forming a carbonate with reactive group O), such as —C(O)—OR^{T1}, where R^{T1} is optionally substituted C₁₋₁₂ alkyl or optionally substituted C₄₋₁₈ aryl, such as methoxycarbonyl, methoxymethylcarbonyl, t-butyloxycarbonyl (Boc), or benzyloxycarbonyl (Cbz); or an optionally substituted silyl group (e.g., forming a silyl ether with reactive group O), such as —Si—(R^{T2})₃, where each R^{T2} is, independently, optionally substituted C₁₋₁₂ alkyl or optionally substituted C₄₋₁₈ aryl, such as trimethylsilyl, t-butyldimethylsilyl, or t-butyldiphenylsilyl. N-protecting groups include, e.g., formyl, acetyl, benzoyl, pivaloyl, t-butylacetyl, alanyl, phenylsulfonyl, benzyl, Boc, and Cbz. Such protecting groups can employ any useful agent to cleave the protecting group, thereby restoring the reactivity of the unprotected reactive group.

By “sulfonium” is meant a group including a protonated sulfur atom S⁺. Exemplary sulfonium groups include —S⁺R^{S1}R^{S2}, where each of R^{S1} and R^{S2} is, independently, H, optionally substituted aliphatic, optionally substituted alkyl, optionally substituted aromatic, optionally substituted aryl, or optionally substituted alkaryl; or R^{S1} and R^{S2} taken together with the sulfur atom to which each are attached, form a heterocyclyl; or R^{S1} and R^{S2}, taken together, form an optionally substituted alkylene or heteroalkylene (e.g., as described herein).

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt).

By “thio” is meant an —S— group

By “thioalkoxy” is meant an alkyl group, as defined herein, attached to the parent molecular group through a sulfur atom. Exemplary unsubstituted thioalkoxy groups include C₁₋₆ thioalkoxy.

By “thiol” is meant an —SH group.

Where values are described in the present document in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

Various embodiments of the features are described herein. However, it should be understood that such embodiments are provided merely by way of example, and numerous variations, changes, and substitutions can occur according to those skilled in the art without departing from the scope described herein. It should also be understood that various alternatives to the specific embodiments described herein are also within the scope described herein.

Other features and advantages will be apparent from the following detailed description, the figures, and the claims.

### BRIEF DESCRIPTION OF THE DRAWINGS

The following drawings illustrate certain embodiments of the features and advantages described herein. These embodiments are not intended to limit the scope of the appended claims in any manner. Like reference symbols in the drawings indicate like elements.

FIG. 1A-1F shows non-limiting examples of devices and components of devices. Provided are non-limiting schematics of (A) a device and a surface of a microchannel; (B) another device and another surface of another microchannel; (C) a system having at least four devices; (D) a surface 101 of a microchannel having a capture agent 130; (E) another surface 102 of a microchannel having a capture agent 132; and (F) a reaction scheme for providing a member 162a of a binding pair to a surface 165.

FIG. 2A-2C shows a non-limiting HB-Chip. Provided is (A) an image of an HB-Chip that is scaled and produced in plastic (left), in which the inner surface of the device includes staggered herringbone grooves that promote chaotic mixing of specimens as they flow through. Also shown is a scanning electron microscopy (SEM) image (middle) of the inner device surface with an inset of exosomes captured on the chip. The inner device surface can be modified, by use of capture agents (right). Also provided is (B) a graph showing optimization of the linker size in the surface coating to maximize capture of small particles. (C) When compared against the most frequently used technologies for extracellular vesicle (EV) isolation, the HB-Chip outperformed both magnetic bead EV isolation (using the same cocktail of antibodies) and ultracentrifugation.

FIG. 3A-3C shows a non-limiting example of device performance as being inversely correlated to target analyte abundance. (A) Microfluidic isolation resulted in >100 fold enrichment of tumor specific RNA using cocktail of antibodies, as compared to the field standard of ultracentrifugation. (B) As the amount of tumor exosomes spiked into plasma was decreased, device performance increased. The chip is highly efficient at capturing particles; and when target vesicles (or virus) are in over abundance, the surface will become saturated, lowering device performance. (C) Also provided are results showing the limit of detection (LOD) as being 100 extracellular vesicles (EVs) in 100 μL or about 1 EV in 1 μL.

FIG. 4A-4B shows the results of (A) a limit of detection (LOD) assay of viral N1 (circle) and N2 (square), as measured by ddPCR. Copies of viral DNA plasmid are shown on the x-axis, and measured copies of the N1 and N2 gene are shown on the y-axis. (B) Pseudoviral RNA extracted in solution were diluted in either PBS (circle) or plasma (square) from 0.1 to 1×10⁻⁶ in a 10× serial dilution

FIG. 5A-5E shows results from on-chip detection using samples spiked with pseudovirus. Provided is (A) a schematic of the experiment for “ACE2” analysis that employs capture of pseudovirus on an HB-Chip and for “Plasma” analysis that does not employ chip-based capture. (B) Capture of pseudoviral particles on the HB-Chip, as compared to bulk plasma extraction. A non-specific negative control (left) was compared to HB-Chip (ACE2) capture (center) as well as bulk extraction in plasma (right) in three independent tests per group. RNA copies were measured using ddPCR after extraction. (C) LoD of RNA copies extracted from bulk plasma (circle) or the same sample processed on the HB-Chip (square). (D) Comparison of two different sources of recombinant ACE2 (square versus circle). Diluted pseudovirus was measured from source 1 only. (E) RNA copies detected in samples isolated on the HB-Chip. Samples were either collected under standard protocol (square) or pre-incubated with capture protein before running on the chip (triangle). Chip-based capture included use of an ACE2 protein (labeled “ACE2”) or an antibody against the spike protein (labeled “αSpike”).

FIG. 6A-6B shows capture of pseudoviral particles on the HB-Chip in (A) plasma or (B) saliva. A non-specific negative control (square) was compared to ACE2 capture (triangle). RNA copies were measured using ddPCR after extraction.

FIG. 7A-7E shows SARS-COV-2 detection using plasma samples spiked with UV-C inactivated SARS-COV-2 viral particles. (A) Different strains (WA1 [circles], B. 1.17 (Alpha) [boxes], and B.1.351 (Beta) [triangles]) of SARS-COV-2 captured using a microfluidic chip. (B) Different strains (WA1 [boxes], Delta [triangles], and Omicron (BA.1) [circles]) of SARS-COV-2 captured using a microfluidic chip. (C) Copies of Delta (gray) and Omicron (black) detected are graphed on the y-axis versus copies spiked (on the x-axis). (D) Copies of Omicron (BA.1) [left] or (BA.5) [right] detected are graphed on the y-axis versus spiked copies from a blinded dilution series on the x-axis. (E) Delta or Omicron (BA.1) variants of SARS-COV-2 or hCoV-229E, hCoV-OC43, Flu-A, Flu-B, RSV-A, or RSV-B were separately spiked into normal plasma and captured by a microfluidic device. ddPCR was then performed to detect either SARS-COV-2 viral RNA or probes specific to each virus bound to the HB-Chip. Detected viral copies were normalized for amount loaded. SARS-COV-2 RNA copies were measured using the Bio-Rad 2019-nCOV CDC ddPCR Triplex Probe Assay after extraction.

FIG. 8A-8B shows use of (A) wild-type ACE2 (labeled “WT ACE2”) or engineered ACE2 (labeled “Eng ACE2”) as capture agents in a chip and (B) LAMP as a detection methodology. (A) Inactivated SARS-COV-2 was spiked into plasma and then flown through microfluidic chips with recombinant wild-type ACE2 (WT ACE2; squares) or an ACE2 variant optimized for higher binding of spike protein (Eng ACE2; triangles) to compare rates of binding. (B) Inactivated SARS-COV-2 was spiked into plasma and then flown through microfluidic chips at the dilutions shown (left). Isolated RNA was measured using the NEB SARS-COV-2 LAMP assay for captured viral samples (left) and with a positive and negative control (right).

FIG. 9A-9C shows SARS-COV-2 detection using a non-limiting HB-Chip. (A) Captured SARS-COV-2 viral particles were subjected to either PBS (squares) or RNase A (triangle) treatment at 37° C. for 30 minutes on chip to determine if RNA was contained inside RNase A protected whole viral particles. Microfluidic chips were rinsed with PBS, and then RNA was extracted and measured by ddPCR. (B) Capture of virus spiked into blood (circles) or from a volume corrected amount of plasma from a matched donor (diamonds) using a microfluidic device. (C) Plasma was collected from COVID+ patients at 3 days post-diagnosis (50 samples), 10-14 days post-diagnosis (14 samples), and 14-18 days post-diagnosis (20 samples).

FIG. 10A-10B shows SARS-COV-2 detection using saliva or stool samples. (A) Approximately 200 μl of saliva was flown through and detected using a microfluidic chip (circles). A corresponding flow through of each sample was also detected using ddPCR (diamonds). (B) Approximately 750 μl of stool diluted in Ficoll-PAQUE Premium was flown through and detected using a microfluidic chip (circles). A corresponding flow through of each sample was also detected using ddPCR (diamonds).

FIG. 11A-11D shows non-limiting examples of chip-based capture of immune vesicles. Provided is (A) a schematic of exosome (“EVs”) isolation from specific cell types using a non-limiting example of an HB-Chip. (B) T-cell exosomes were captured on an HB-Chip using either control antibody (IgG) or an anti-CD4 antibody. RNA was isolated from vesicles captured on the chip, and ACTB levels were measured using ddPCR. (C) Using clinical samples, exosomes were captured from 150 μL of patient plasma using an anti-CD4 antibody to measure T-cell vesicles. (D) Exosomes were captured from patient plasma using a cocktail of anti-EpCAM and anti-EGFR antibodies to capture epithelial exosomes.

FIG. 12A-12D shows non-limiting examples of chip-based capture of immune vesicles. (A) Schematic showing comparison of “Immune EV” capture antibody cocktail microfluidic devices compared to IgG “Negative Control” microfluidic devices. (B) Neutrophil EVs from HL-60 cells spiked into plasma and captured using a microfluidic devices. Shown are three independent chips with either an IgG negative control, CD11b, or CD66b capture. Neutrophil markers are measured by ddPCR and RNA copies are then z-score normalized across different capture chips. (C) T-Cell EVs from SupT-1 and Jurkat cells spiked into plasma and captured using microfluidic devices. Shown are four independent chips with either an IgG negative control or T-cell capture cocktail (CD3, CD4, and CD8 capture). T-cell markers are measured by ddPCR and RNA copies are then z-score normalized across different capture chips. (D) Macrophage EVs from THP-1 cells spiked into plasma and captured using microfluidic devices. Shown are either an IgG negative control, CD11b, or CD66b capture. Macrophage markers are measured by ddPCR and RNA copies are then z-score normalized across different capture chips.

FIG. 13A-13B shows non-limiting examples of chip-based capture of immune vesicles. Provided is (A) a schematic of exosome (“EVs”) isolation from specific cell types using a non-limiting example of an HB-Chip. (B) T-Cell EVs from Jurkat cells spiked into plasma and captured using microfluidic devices. Shown are four independent chips with IgG negative control (circles), CD3 (squares), CD4 (triangles), or CD8 (diamonds) capture. T-cell markers are measured by ddPCR.

FIG. 14 shows decrease in T-cell EVs in COVID+ patients. Four healthy donor plasma samples and four samples from COVID-19+ patients were flown through a microfluidic device for T-Cell exosome (EV) capture. RNA levels of CCL5, ACTB, CD3, and CD45 were measured for each chip using ddPCR.

FIG. 15 shows that severe COVID+ patients have a different EV signature. Samples were collected at 14-18 days post (+) from 20 patients, in which 250 μL of banked plasma was tested. Disease severity included a rating scale of 1 to 4, which included 1—Mild (patients who do not require inpatient hospitalization), 2—Moderate (hospitalized patients who do not require more than 15 LPM of supplemental oxygen and who do not have organ failure), 3—Severe (hospitalized patients who have organ failure, typically in an ICU, such as high flow oxygen, NIPPV, mechanical ventilation, vasopressors, renal replacement therapy), and 4—Deceased. RNA copies were z-score normalized across patients and arranged by outcome severity scale. T-Cell EVs show higher signal in patients with higher severity scores.

FIG. 16A-16C shows that severe COVID+ patients have a different EV signature. (A) For the same set of patients in FIG. 15 and with an additional six healthy donor samples, T-Cell, Epithelial, and Innate Immune microfluidic chips were used to capture these different EV types from each single patient sample. RNA levels were z-score normalized across patients and healthy donors. T-cell EVs show much higher RNA levels in healthy donors compared to COVID patients. By comparison Epithelial and Innate EVs show higher signal only in severe outcome (severity 3-4) patients. (B) Four saliva samples from healthy donors and nine from COVID+ patients were run across T-Cell, Epithelial, and Innate Immune EV capture chips. RNA levels were z-score normalized across individuals showing increased levels in COVID-19+ patient saliva EVs compared to healthy saliva EVs. (C) Four stool samples from COVID+ patients were run across T-Cell, Epithelial, and Innate Immune EV capture chips. RNA levels were z-score normalized across individuals showing some EV RNA signatures in stool similar to saliva and plasma derived EVs.

FIG. 17A-17C shows that severe COVID+ patients have a different EV signature. Provided is (A) a schematic of exosome (“EVs”) isolation from specific cell types using a non-limiting example of an HB-Chip. (B) Fourteen plasma samples from COVID+ patients were run across T-Cell, Epithelial, and Innate Immune EV capture chips. RNA levels were z-score normalized across individuals, showing higher EV signatures across all three types in severe outcome patients specifically. (C) Twenty plasma samples from COVID+ patients were run across T-Cell, Epithelial, and Innate Immune EV capture chips. RNA levels were z-score normalized across individuals, showing higher EV signatures across all three types in severe outcome patients specifically.

FIG. 18A-18C shows viral load compared to clinical metrics. (A) SARS-COV-2 copies detected in plasma are shown for patients with (squares) or without (circles) obesity, hypertension, admitted to the ICU, or given supplemental oxygen (from left to right). (B) SARS-COV-2 copies detected in plasma are shown for patients with (squares) or without (circles) subsequent treatment with dexamethasone or remdesivir. On the right is a contingency table for the predictive power of positive COVID-19 plasma levels and whether they would receive remdesivir. (C) SARS-COV-2 copies detected in saliva are shown for patients with (squares) or without (circles) subsequent treatment with remdesivir

FIG. 19A-19C shows the use of cell-specific EV capture for determining response to immunotherapy drugs. (A) Schematic showing serial capture of melanoma as well as immune EVs from the same plasma sample to derive gene enrichment signatures from patients that do or do not respond to immunotherapy. (B) CIBERSORT-inferred deconvolution estimates for all pretreatment patient tumor and pretreatment patient plasma-derived EV samples using LM22 immune reference profiles. Technical replicates were averaged, and biological replicates were considered independently. The data are segregated into three categories based on the results of a Mann-Whitney U test between EV- and tumor-inferred CIBERSORT fractions for each deconvolved cell type. (C) Five plasma samples from melanoma patients and one healthy donor (HD) were run across T-Cell, B-Cell, and Innate Immune EV capture chips. RNA levels were z-score normalized across individuals, showing higher EV signatures in two patients across all three EV subtypes.

FIG. 20 shows a non-limiting aryl-diazonium reaction strategy. (A) Reaction strategy for aryl-diazonium functionalization. (B-E) Schematic comparing different methods of functionalizing the surface of herringbone devices to allow binding of biotinylated antibodies. (B) Biotin-aryl-diazonium coated plastic devices with streptavidin nanoparticles. (C) Physisorption of NeutrAvidin to plastic devices. (D) Silane-GMBS treatment of PDMS devices with NeutrAvidin (E) Biotin-aryl-diazonium coated PDMS devices with streptavidin nanoparticles.

FIG. 21 shows that aryl-diazonium functionalization decreases with concentration. (A) Early experiments were conducted by first reacting aryl diazonium with the surface of the chip using various concentrations ranging from 10 to 500 μM. Following deposition of aryldiazonium to the chip surface, NHS-ester biotin was reacted within the chip to the surface, and the binding capacity measured using an R-PE assay. Increasing amounts of aryl-diazonium led to a decrease in binding capacity. N=1 chip per concentration and 9 measurements per chip. (B) A visual inspection of the chips following treatment showed a large deposition of aryl-diazonium (brown, 500 μM, top) that clogged the chips and leading to less deposition of biotin on the surface. Images taken with an iPhone.

FIG. 22 shows optimization of a non-limiting aryl-diazonium reaction. (A) Schematic depicting the Biotin-(RPE) assay for determining binding capacity of functionalized devices. (B-F) Average fluorescent RPE intensity is shown for nine areas per device functionalized. (B) Devices were functionalized for 15 minutes using a UV light bed at high (red) or a UV light box with differing energies: 100 mJ cm⁻², 200 mJ cm⁻², or 400 mJ cm⁻²; (C) UV light box set at high for 5 minutes, 10 minutes, or 15 minutes; (D) a biotin to aryl-diazonium ratio of 1:2, 1:1, or 2:1; (E) with streptavidin nanoparticles or neutravidin; or (F) reaction volume of 200 μL flown through the device twice, 200 μL flown through the device once, or 100 μL flown through the device once. For all experiments (N=3), devices were used per condition. Average fluorescent intensity values are shown for (N=9) images across each device surface. P-values were determined using a two-way ANOVAs, with correction for multiple comparisons (B-D,F) or a one-way ANOVA (E).

FIG. 23 shows representative images from UV light bed versus UV light box functionalization. Devices were functionalized for 15 minutes using a UV light bed at high (top left) or a UV light box with differing energies: 100 mJ cm⁻² (top right), 200 mJ cm⁻² (bottom left), or 400 mJ cm⁻² (bottom right). All images were taken for 100 ms using a 10× lens. UV light box treatment, particularly at higher energies, showed many dark patches of un-functionalized surface. Because the UV light bed showed better more consistent quality, it was used moving forward.

FIG. 24 shows representative images for different times exposed to UV light. Devices were functionalized for 5 minutes (top), 10 minutes (middle), or 15 minutes (bottom) using a UV light bed set to high. No difference was seen between 10 and 15 minutes, so 10 minutes was used moving forward. All images were taken for 100 ms using a 10× lens.

FIG. 25 shows representative images for different NHS-biotin to aryldiazonium ratios. Devices were functionalized for 15 minutes using a UV light bed using either a reaction ratio of 1:2 (A), 1:1 (B), or 2:1 (C) of biotin to aryl-diazonium. All images were taken for 100 ms using a 20× lens. No difference was seen between 1:1 and 2:1 ratio, so 1:1 ratio was used moving forward.

FIG. 26 shows representative images from chips with either Streptavidin nanoparticles or NeutrAvidin. Devices were functionalized as described herein, followed by addition of Streptavidin nanoparticles or NeutrAvidin (five device volumes of 20 μg/mL NeutrAvidin through the inlet, then the outlet). All images were taken for 100 ms using a 20× lens. Streptavidin nanoparticles showed an increase fluorescent signal compared to NeutrAvidin and were used for all subsequent experiments.

FIG. 27 shows representative images from chips functionalized with different amounts of biotin aryl-diazonium solution of twice with 200 μL (A), once with 200 μL (B), and once with 100 μL (C). All images were taken for 100 ms using a 20× lens. One device volume resulted in dark spots with no biotin (likely where NO₂ bubbles formed during initial UV treatment). Two device volumes (200 μL) of solution flown through the chips twice showed the most consistent results and was used moving forward.

FIG. 28 shows device stability. For each device, average fluorescent intensity values are shown for (N=9) images across the device surface. RPE intensity is shown for devices functionalized and stored (A) in PBS at 4° C. (circle), PBS at 25° C. (square), or dry in desiccant at 25° C. (triangle) (N=1 device per time point, per condition), (B) dry in desiccant at 25° C. for up to 29 days (N=6 per time point), and (C) dry in a vacuum desiccator at 25° C. for up to six months (N=5 per time point). P-values were determined using a two-way ANOVA with correction for multiple comparisons.

FIG. 29 shows representative images from devices stored in desiccant at 25° C. for up to four weeks. Devices were stored in a desiccator box over four weeks. All images were taken for 100 ms using a 10× lens. A 29% drop is seen in chips stored at week one versus week zero. No additional degradation was seen in the following weeks. Devices were stored in desiccant (for shorter term storage) or in a vacuum desiccator (for longer term storage).

FIG. 30 shows representative images from devices stored in a vacuum desiccator at 25° C. for up to six months. Devices were stored in a vacuum desiccator box until use for up to six months. All images were taken for 100 ms using a 10× lens. No degradation was seen over a six-month period when stored in a vacuum desiccator.

FIG. 31 shows that aryl-diazonium devices have similar binding compared to other methods. For each device, average fluorescent intensity values are shown for (N=9) images across the device surface. RPE intensity is shown for (A) plastic devices functionalized by physisorption of neutravidin (left) or by aryl-diazonium and streptavidin nanoparticles (right); (B) PDMS devices functionalized with silane, N-(γ-maleimidobutyryloxy) succinimide ester (GMBS), and neutravidin (squares) or with aryl-diazonium and streptavidin nanoparticles (triangles); or (C) PDMS devices functionalized with silane, GMBS, and neutravidin (squares) or plastic devices functionalized aryl-diazonium and streptavidin nanoparticles (circles). For all experiments (N=4), devices were used per condition; and P-values were determined using an unpaired t-test.

FIG. 32 shows representative images from different device types and functionalization strategies. All images were taken for 100 ms using a 10× lens.

FIG. 33 shows that aryl-diazonium devices bind tumor EVs at a higher rate. (A) Schematic of experimental setup. Concentrated tumor EVs are flown through the device with a syringe pump. EVs captured on microfluidic devices are then detected by ddPCR following RNA extraction. (B-C) Concentrated serum-free conditioned media containing tdTomato labelled EVs were flown through and captured on devices containing an IgG antibody (B) or an anti-EGFR antibody (Cetuximab). (C) RNA was extracted and measured by ddPCR with (N=3) devices per condition.

FIG. 34A-34B shows results of determining fluorescent intensity of EVs captured on the HB-Chip. Concentrated serum-free conditioned media containing palm-tdTomato labelled EVs from either MDAMB-231-BM1 were flown through and captured on devices containing an IgG antibody or an anti-EGFR antibody (Cetuximab). (A) Fluorescent intensity of captured palm-tdTomato+ EVs was measured by taking nine images at 10× zoom for one second. P-values were calculated using a two-way ANOVA with correction for multiple comparisons. (B) Representative images of each chip type with IgG or EGFR capture.

FIG. 35 shows that aryl-diazonium devices specifically capture tumor EVs from plasma. (A) Schematic of experimental setup. Concentrated tumor EVs or PBS are spiked into normal plasma and then flown through the device with a syringe pump. EVs captured in microfluidic devices are then detected by ddPCR following RNA extraction. (B-C) Concentrated serum-free conditioned media containing tdTomato labelled EVs from either MDA-MB-231-BM1 (B) or MDA-MB-468 (C) tumor cells were spiked into normal plasma, then flown through and captured on devices containing an IgG antibody or an anti-EGFR antibody (Cetuximab). RNA was extracted and measured by ddPCR.

FIG. 36 shows results of determining fluorescent intensity of EVs in plasma captured on the HB-Chip. Concentrated serum-free conditioned media containing palm-tdTomato labelled EVs from either MDA-MB-231-BM1 (A) or MDA-MB-468 (B) tumor cells or PBS (Control) were spiked into normal plasma, then flown through and captured on devices containing an IgG antibody or an anti-EGFR antibody (Cetuximab). Fluorescent intensity of captured palmtdTomato+ EVs was measured by taking nine images at 10× zoom for one second. P-values were calculated using a two-way ANOVA with correction for multiple comparisons. *These devices were measured using a 20× lens resulting in lower fluorescent intensity compared to other devices.

FIG. 37 shows representative images of fluorescent intensity of EVs in plasma captured on the chip. Concentrated serum-free conditioned media containing palm-tdTomato labelled EVs from either MDA-MB-231-BM1 (A) or MDA-MB-468 (B) tumor cells or PBS (Control) were spiked into normal plasma, then flown through and captured on devices containing an IgG antibody or an anti-EGFR antibody (Cetuximab). Fluorescent intensity of captured palm-tdTomato+ EVs was measured by taking nine images using a 10× lens with one second capture.

FIG. 38A-38B shows a comparison of surfaces treated with gelatin or aryl diazonium. Concentrated serum-free conditioned media (with or without tdTomato labelled EVs) were flown through and captured on devices treated with gelatin or aryl diazonium, in which the surface can further contain an IgG antibody or an anti-EGFR antibody. (A) Fluorescent intensity was measured for each surface. (B) RNA was extracted and measured by ddPCR for the provided conditions.

### DETAILED DESCRIPTION

The present document relates to devices, systems, and methods of capturing a target. In some embodiments, the target is captured in an intact form (e.g., an intact particle, vesicle, cell, and the like) from a liquid sample. Capture can include the use of a microfluidic device, which in turn can include a microchannel, as well as an inlet and an outlet in fluidic communication with the microchannel. The inlet can be configured to deliver a sample to the microchannel, and the outlet can be configured to provide a captured target or a portion of the sample in which the target has been depleted (e.g., as compared to the sample provided to the inlet).

The microchannel can include any surface, including a structure disposed on the surface to modify fluid flow within the channel or a functionalized surface to capture the target. For example and without limitation, the structure can include at least one groove, which is defined in the inner wall surface of the microchannel. In some embodiments, a plurality of grooves can be arranged and configured to generate chaotic flow within a fluid sample traveling through the microchannel. The functionalized surface can include one or more capture agents configured to capture the target. Such capture agents, in turn, can be attached directly or indirectly to a surface of the microchannel. Indirect attachment can include the use of linkers, particles, and/or binding pairs (e.g., any described herein).

The microchannel can be disposed within a microfluidic device. A device can include a monolithic structure or a modular structure, in which a microchannel is defined in at least one substrate. In some embodiments, the device can include a first substrate having a conduit defined therein and a second substrate having a planar surface. In turn, the planar surface can include a groove, a plurality of grooves, or other structures to provide chaotic flow when the first and second substrates are contacted together to form a microchannel. For instance, the microchannel can be formed when the conduit and the planar surface, together, form an enclosed region that can transport fluid.

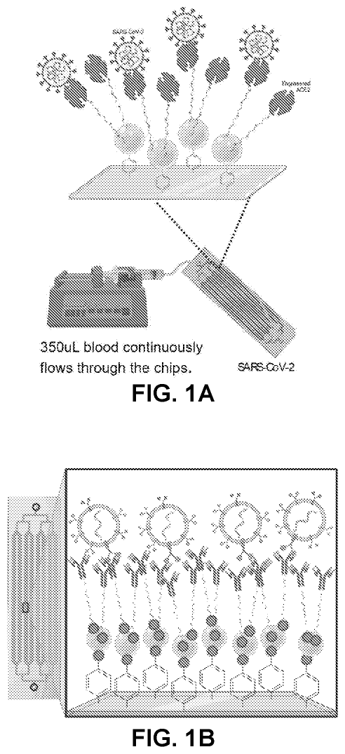

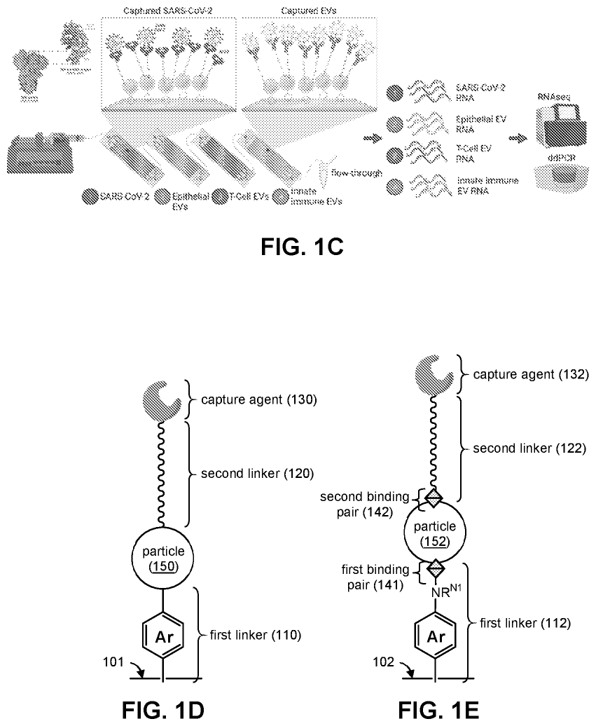

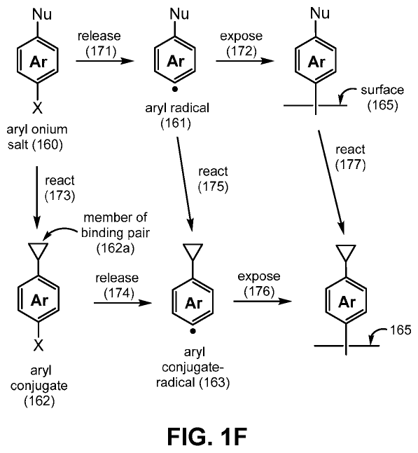

FIG. 1A shows a schematic of a non-limiting device. As can be seen, the microchannel can include an inner wall surface having a plurality of capture agents (here, e.g., the capture agents include engineered ACE2). The capture agent can be used to capture a target (here, e.g., the targets include severe acute respiratory syndrome coronavirus 2 (SARS-CoV-2)). Furthermore, the capture agent can be attached the inner wall surface using a first linker (e.g., a phenylene group), a particle, and a second linker (e.g., a poly (ethylene glycol) (PEG) linker). As can be seen, a plurality of capture agents can be attached to each particle. In this way, the density of capture agents can be increased, as compared to using a single linker attached a single capture agent.

FIG. 1B shows a schematic of another non-limiting device. Here, the capture agent can include an antibody, which can be used to capture a vesicle displaying a target that binds the antibody. Here too, linkers and particles can be used to control the density of the capture agents provided within the inner surface of the microchannel. Any useful binding pairs may be used to form bonds between any combination of the capture agent, linker, and particle. Additional capture agents, targets, linkers, particles, and binding pairs are described herein.

A system can include one, two, three, or more microfluidic devices. When a plurality of microfluidic devices are present, fluidic interconnects can be used to contact an outlet of a first device to an inlet of a second device. In this way, devices can be serially connected to provide a fluidic network. In other embodiments, devices can be connected in parallel, in which a sample can be divided into separated samples of smaller volumes. Such separated samples can then be delivered to other devices. The system can include other components to control fluid flow (e.g. to transport a sample into one or more microchannels), release targets from the microchannels, collect fluid samples from microchannel(s), and/or analyze targets (e.g., on-chip or off-chip).

FIG. 1C shows a schematic of a non-limiting system. As can be seen, each device can be configured to capture a particular target, and a plurality of devices can be fluidically connected in a serial manner. Each device includes an inlet and an outlet, and a fluidic interconnect can be provided between an inlet and an outlet of different devices. For instance, a first device can include a first inlet and a first outlet, in which the inner wall surface of a first microchannel of the first device is configured to capture a first target. In addition, a second device can include a second inlet and a second outlet, in which the inner wall surface of a second microchannel of the second device is configured to capture a second target. A first fluidic interconnect can be present between the first outlet and the second inlet to provide fluidic communication between the first and second microchannels. In this way, the first target-depleted sample from the first outlet is delivered to the second inlet of the second device. After passage through the second microchannel, the resultant sample will be second target-depleted sample. In a similar manner, further devices can be used to capture further targets.

As further shown in FIG. 1C, the inner wall surface of the first device can include a capture agent configured to capture a first target (here, e.g., the first target includes severe acute respiratory syndrome coronavirus 2 (SARS-COV-2)). After passage through the first device, the resulting sample can be a first target-depleted sample. In turn, the first target-depleted sample can be used provided to an inlet of a second device (e.g., a second inlet of the second device). In this way, a second target can be captured from the sample (here, e.g., the second target includes epithelial extracellular vesicles). Further devices can be used to capture further targets. For example, a third device is configured to capture a third target (here, e.g., the third target includes T-cell extracellular vesicles), and a fourth device is configured to capture a fourth target (here, e.g., the fourth target includes innate immune extracellular vesicles). For each device, a different capture agent can be used to capture different targets, and any useful combination of linkers, particles, and/or binding pairs may be used to attach capture agents and provide a functionalized surface within the microchannel.

After capture, the targets can be released from the microchannel in any useful manner. For instance, release can include lysing or otherwise breaking apart intact viruses, particles, cells, or vesicles to access internal components (e.g., nucleic acid, proteins, etc.). Lysing can include the use of lysing agents (e.g., detergents, salts, chaotropes, etc.), heat, pressure (e.g., acoustic pressure), ultrasound, and the like. If desired, a solvent can be used to collect the lysed components. Any resulting lysate can be further analyzed (e.g., by amplification, sequencing, and the like). When a system includes a plurality of devices, each device can be optionally fluidically disconnected prior to release of the captured targets.

Also described herein are methods of making a device or a system. In some non-limiting embodiments, the device can include a particular linker, which can be attached to a substrate for forming the microchannel. When the linker includes an arylene moiety, aryl-onium salts may be used to functionalize the surface. Reactions that employ such salts can be readily applied to various substrate, including glass and even plastic. In some embodiments, the functionalized surfaces can exhibit improved stability and capture ability, as compared to surfaces that do not employ such chemistry. Details of using aryl-onium salts are described herein, and the present document encompasses the use of such chemistry to provide functionalized surfaces in microchannels.

Yet also described herein are methods that employ any device or system described herein. Non-limiting methods include methods of isolating a virus in a sample, methods of determining a viral load in a sample, and methods of capturing a target. These methods can include providing any device or system described herein, as well as one or more other operations, such as, e.g., flowing or delivering a sample (e.g., a test sample, a collected sample, or other sample) to a microchannel, preparing a sample (e.g., diluting and/or stabilizing the sample) prior to delivery of the sample to the microchannel, capturing a target in the microchannel, lysing the captured target within the microchannel, analyzing a lysate (e.g., including one or more lysed components from the captured target), collecting a sample (e.g., after flowing through a microchannel), determining a characteristic (e.g., a concentration, a sequence, and the like) of the captured target (e.g., on-chip or off-chip), and/or determining a characteristic (e.g., a concentration, a sequence, and the like) of the released target (e.g., on-chip or off-chip).

### Devices and Systems

The present document relates to devices, as well as systems having one or more devices. The device can include one or more structures defined in an inner wall surface of the microchannel. The structure can include any arranged and configured to generate chaotic flow within a fluid sample traveling through the microchannel. In some embodiments, the structure can include one or more (e.g., or a plurality of) grooves (e.g., V-shaped grooves), ridges, posts, staggered herringbones, or a combination of any of these. Non-limiting devices, microchannels, and structures are described in U.S. Pat. Nos. 11,548,002, 10,551,376, 10,126,218, and 10,0186,32, each of which is incorporated herein by reference in its entirety.

The device can include one or more capture agents. In some embodiments, the capture agent is disposed on the inner wall surface having the one or more structures described herein (e.g., grooves, staggered herringbones, etc.). In other embodiments, the capture agent is disposed on the inner wall surface lacking such structures (e.g., a flat inner wall surface).

The capture agent can be attached to the surface by way of one or more linkers, particles, binding pairs, or a combination of any of these. FIG. 1D shows a schematic of a non-limiting functionalized surface having a capture agent. Non-limiting examples of capture agents include proteins, aptamers, as well as any described herein. As can be seen, a first linker 110 can be attached to the inner wall surface (or a portion thereof) 101 (herein, e.g., the first linker comprises an arylene moiety Ar). A first linker can attached directly or indirectly to a capture agent.

In one embodiment, the first linker is indirectly attached to the capture agent. As seen in FIG. 1D, a particle 150 can be attached to the first linker 110. Optionally, a second linker 120 can be attached to the particle 150 and to the capture agent 130. In some embodiments, the particle can include a plurality of second linkers, in which each second linker can be attached to a capture agent. Examples of linkers are described herein.

In some embodiments, the linker (e.g., a first linker, a second linker, or another linker) is a covalent bond, oxy (e.g., —O—), thio (—S—), imino (e.g., —NR^{N}—, wherein R^{N1} is hydrogen (H), optionally substituted aliphatic, or optionally substituted alkyl (e.g., C₁₋₆ alkyl)), carbonyl (e.g., —C(O)—), optionally substituted aliphatic, optionally substituted alkylene, optionally substituted heteroaliphatic, optionally substituted heteroalkylene, optionally substituted aromatic, or optionally substituted arylene.

A linker can be attached to an inner wall surface, or a portion thereof, of the microchannel. In some embodiments, the linker can include an arylene moiety, as described herein. In some embodiments, the arylene moiety is phenylene (e.g., -Ph-). The arylene moiety can be attached to the surface in any useful manner. In some embodiments, the arylene moiety is provided in a para position on the aromatic ring. In some embodiments, the arylene moiety includes a benzene ring, in which the ring is attached to the inner wall surface at a 1-position within the ring, and in which the ring is attached (directly or indirectly) to a capture agent at a 4-position within the ring. In other embodiments, the arylene moiety is or includes para-phenylene.

A linker can also be attached to the capture agent. In one example, a single linker can be attached between the inner wall surface and the capture agent. In another example, a first linker is attached to the inner wall surface, and a second linker is attached to a capture agent. The first and second linkers can be directly attached to each other or indirectly attached to each other (e.g., by way of another linker, a particle, a bead, or another component).

The linker (e.g., a first linker, a second linker, or another linker) can include one or more arylene moieties. In some embodiments, the linker includes —Ar—, wherein Ar is optionally substituted arylene (e.g., as described herein). In other embodiments, the linker includes —Ar—Z—, wherein Ar is optionally substituted arylene, and wherein Z is a covalent bond, hydrogen (H), oxy (e.g., —O—), thio (—S—), imino (e.g., —NR^{N}—, wherein R^{N1} is hydrogen (H), optionally substituted aliphatic, or optionally substituted alkyl (e.g., C₁₋₆ alkyl)), carbonyl (e.g., —C(O)—), optionally substituted alkylene, or optionally substituted heteroalkylene. In yet other embodiments, the linker includes —Ar—NR^{N1}—, wherein Ar is optionally substituted arylene, and wherein R^{N1} is hydrogen (H), optionally substituted aliphatic, or optionally substituted alkyl (e.g., C₁₋₆ alkyl).

In some embodiments, the linker includes —Ar—, wherein Ar is optionally substituted arylene (e.g., as described herein). In other embodiments, the linker includes —Ar—Z—, wherein Ar is optionally substituted arylene, and wherein Z is a covalent bond, hydrogen (H), oxy (e.g., —O—), thio (—S—), imino (e.g., —NR^{N}—, wherein R^{N1} is hydrogen (H), optionally substituted aliphatic, or optionally substituted alkyl (e.g., C₁₋₆ alkyl)), carbonyl (e.g., —C(O)—), optionally substituted alkylene, or optionally substituted heteroalkylene. In yet other embodiments, the linker includes —Ar—NR^{N1}—, wherein Ar is optionally substituted arylene, and wherein R^{N1} is hydrogen (H), optionally substituted aliphatic, or optionally substituted alkyl (e.g., C₁₋₆ alkyl).

The linker (e.g., a first linker, a second linker, or another linker) can include optionally substituted aliphatic, alkylene, heteroaliphatic, heteroalkylene, aromatic, or arylene. In particular embodiments, the linker is a flexible linker. Non-limiting examples of linkers include a bond (e.g., a covalent bond), optionally substituted alkylene, optionally substituted heteroalkylene (e.g., poly(ethylene glycol)), optionally substituted arylene, and optionally substituted heteroarylene. Other non-limiting examples of linkers include dextran. Yet other non-limiting examples of linkers can include an ethylene glycol group, e.g., —OCH₂CH₂—, including a poly (ethylene glycol) (PEG) group —(OCH₂CH₂)ₙ—, a four-arm PEG group (such as C[CH₂O(CH₂CH₂O)ₙ—]₄ or C[CH₂O(CH₂CH₂O)ₙCH₂—]₄ or C[CH₂O(CH₂CH₂O)ₙCH₂CH₂—]₄), an eight-arm PEG group, or a derivatized PEG group (e.g., methyl ether PEG (mPEG), a propylene glycol group, etc.); including dendrimers thereof, copolymers thereof (e.g., having at least two monomers that are different), branched forms thereof, start forms thereof, comb forms thereof, etc., in which n is any useful number in any of these (e.g., any useful n to provide any useful number average molar mass Mn). In some embodiments, the linker has a molecular weight of about 1.8 to 4.8 kDa.

In some embodiments, the flexible linker has a molecular weight between approximately 1.0 to 5.0 kDa, e.g., 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.75, 4.0, 4.25, 4.5, 4.75, or 5.0 kDa. The flexible linker can be made of PEG, dextran, or any other suitable polymer that is capable of binding to the inner wall surface and the linker, binding pair, particle, and/or capture agent.

Optionally, a particle can be present between the inner wall surface and the capture agent. The particle can have any useful shape or geometry. Furthermore, the particle can include a bead, a fiber, a core-shell structure, a nanoparticle, a microparticle, a quantum dot, and the like. The particle can be formed of any useful material, including a polymer (e.g., any described herein), a semiconductor material, a metal, a glass, a ceramic, a protein, a saccharide, and the like, as well as combinations thereof. In some embodiments, the particle can include a member of a binding pair.

One or more binding pairs can be used between components to form an attachment. Non-limiting binding pairs include biotin and avidin, biotin and streptavidin, biotin and neutravidin, desthiobiotin and avidin (or a derivative thereof, such as streptavidin or neutravidin), hapten and an antibody, an antigen and an antibody, a primary antibody and a secondary antibody, a carbohydrate binding protein and a carbohydrate, and lectin and a glycoprotein. Other binding pairs can include, e.g., histidine and nickel, glutathione S-transferase (GST) and glutathione, maltose binding protein (MBP) and maltose, fluorescein isothiocyanate (FITC) and anti-FITC, c-myc-tag and anti-c-myc, human influenza hemagglutinin (HA) and anti-HA. Each component within a binding pair can be considered a member (e.g., a first member and a second member within a binding pair).

In some embodiments, the binding pair includes a click-chemistry reaction pair. Each component within a binding pair can be considered a member (e.g., a first member and a second member within a binding pair). Non-limiting click-chemistry reaction pairs include those selected from the group consisting of a Huisgen 1,3-dipolar cycloaddition reaction between an alkynyl group and an azido group to form a triazole-containing linker; a Diels-Alder reaction between a diene having a 47 electron system (e.g., an optionally substituted 1,3-unsaturated compound, such as optionally substituted 1,3-butadiene, 1-methoxy-3-trimethylsilyloxy-1,3-butadiene, cyclopentadiene, cyclohexadiene, or furan) and a dienophile or heterodienophile having a 27 electron system (e.g., an optionally substituted alkenyl group or an optionally substituted alkynyl group); a ring opening reaction with a nucleophile and a strained heterocyclyl electrophile; and a splint ligation reaction with a phosphorothioate group and an iodo group; and a reductive amination reaction with an aldehyde group and an amino group.

In other embodiments, binding pairs can include the following: antibodies, antibody fragments (e.g., Fc fragments), oligo- or polypeptides, nucleic acids, cellular receptors, ligands, aptamers, MHC-peptide monomers or oligomers, biotin, avidin, oligonucleotides, coordination complexes, synthetic polymers, and carbohydrates. Members of binding pairs can be attached to linkers, particles, and/or capture agents using methods known in the art. For example, biotinylation of antibodies can be accomplished through multiple routes by one skilled in the art, by reacting with the various moieties present, including but not limited to primary amines, sulfhydryl groups, and carboxyl groups. These routes can be either chemical or enzymatic and are typically mediated by a reactive group attached to the binding agent, e.g., biotin. The methods employed will depend on the binding pair, reactive groups, and the like.

Binding pairs can be employed to attach one or more linkers. FIG. 1E shows a schematic of a non-limiting functionalized surface using binding pairs. As can be seen, a first linker 112 can be attached to the inner wall surface (or a portion thereof) 102 (herein, e.g., the first linker comprises an arylene moiety Ar). In addition, a particle 152 can be attached to the first linker 112, and a second linker 122 can be attached to the particle 152 and to the capture agent 132. Furthermore, a first binding pair 141 can be present between the first linker 112 and the particle 152, and a second binding pair 142 can be present between the second linker 122 and the particle. The first and second binding pairs can be same or different. The first and second binding pairs can be any described herein.

For the device, the substrate can be formed from any useful material. In one instance, the substrate or device includes a semiconductor material (e.g., silicon, silicon oxide, silicon nitride, etc.). In another instance, the substrate or device includes a polymer (e.g., a functionalized polymer). Exemplary polymers includes cyclic olefin polymer (COP), cyclic olefin copolymer (COC), polynorbornene, off-stoichiometry thiol-ene (OSTE), off-stoichiometric thiol-ene-epoxy (OSTE+), polymethylmethacrylate (PMMA), polycarbonate (PC), poly (bisphenol A carbonate), poly (propylene carbonate), polystyrene (PS), styrene copolymer, polyethylene terephthalate (PET, e.g., biaxially-oriented PET or bo-PET), an acrylic polymer, poly (dimethylsiloxane) (PDMS), polyethylene terephthalate glycol (PETG), polyethylene (PE, such as branched homo-polymer PE), polyvinylchloride (PVC), polyimide (PI), polypropylene (PP), polyester, polytetrafluoroethylene (PTFE), poly (4-methyl-1-pentene), silicone, and combinations or co-polymers thereof. Polymers can include any useful additive, such as, e.g., photoinitiators, curing agents, fillers (e.g., mica, talc, or calcium carbonate), plasticizers (e.g., dioctyl phthalate), heat stabilizers (e.g., organo-tin compounds), antioxidants (e.g., phenols or amines), and/or UV stabilizers (e.g., benzophenones or salicylates).

The device can include one or more other components, such as a coating (e.g., including a surface layer of gelatin), and the like. Non-limiting devices, microchannels, components, and structures are described in U.S. Pat. Nos. 11,548,002, 10,551,376, 10,126,218, and 10,0186,32, each of which is incorporated herein by reference in its entirety.

The systems herein can include one or more devices, in which each device can include one or more microchannels. When a plurality of devices are present, the devices can be connected in series or in parallel. Fluidic communication between devices can be provided by way of fluidic interconnects. Non-limiting examples of fluidic interconnects include a tubing, a pipe, a channel, and the like.

### Methods of Preparing a Device

The present documents encompasses methods of preparing a device. In one embodiment, the method employs aryl-onium salts to form covalent bonds with a surface of a device, microchannel, or substrate. An aryl-onium salt is an intermediate having reactive groups, which in turn can react with a surface and another component (e.g., a linker, a particle, or a capture agent) to form a covalent bond. The intermediate also includes an aromatic moiety (e.g., an optionally substituted aromatic, aryl, or arylene, as described herein) to which the reactive groups are attached.

The aryl-onium salt can be characterized as having a first reactive group and a second reactive group. In use, the first reactive group can include an onium group, which can be easily removed to form a free radical. This free radical can then react with another functional group, such as those present on a surface of the microchannel or on a surface of the substrate. The second reactive group can include a functional group that can react with a linker, a particle, or a capture agent. In some embodiments, the second reactive group can include a nucleophilic group or others described herein.

Non-limiting examples of reactive groups (e.g., a first reactive group, a second reactive group, or another reactive group) include an onium group, such as an ammonium cation, a diazonium cation, a halonium cation, an oxonium cation, a phosphonium cation, or a sulfonium cation, as described herein; a nucleophilic group, such as an amino (e.g., —NR^{N1}R^{N2}, wherein each of R^{N1} and R^{N2} is, independently, hydrogen or C₁₋₆ alkyl), a thio group (e.g., —SH), a hydroxyl group (e.g., —OH), an anion, and the like.

In some embodiments, the aryl onium salt has a structure of formula (Ia):

X₁—Ar—X₂ (I) or a salt thereof, wherein:
Ar is or comprises an optionally substituted aromatic or optionally substituted arylene; and
each of X₁ and X₂ is, independently, a reactive group (e.g., any described herein).

In some embodiments, at least one of X₁ and X₂ is or comprises an onium group.

In some embodiments, the aryl onium salt has a structure of formula (Ia):

Nu—Ar—X (I) or a salt thereof, wherein:
Nu is or comprises a nucleophilic group (e.g., any described herein);
Ar is or comprises an optionally substituted aromatic or optionally substituted arylene; and
X is or comprises an onium group.

In some embodiments, the onium group (X) and the nucleophilic group (Nu) are provided in a para position.

The method can include forming an aryl-onium salt comprising an arylene moiety disposed between an onium group and a nucleophilic group; releasing the onium group to form an aryl radical; and exposing the aryl radical to a surface of a microfluidic device, thereby providing a functionalized surface. The forming, releasing, and exposing operations can be conducted in any useful manner and in any useful order to provide a desired attachment between the arylene moiety and the surface of the microchannel.

The aryl-onium salt can be formed in any useful manner. In one non-limiting embodiment, the aryl-onium salt is formed by exposing an arylene compound to reactant. In some embodiments, the reactant can be an oxidant (e.g., nitrous acid, a nitrite salt, a peroxycarboxylic acid, etc.), an acid (e.g., hydrogen halide), an alkylating agent, and the like. The arylene compound can include one or more functional groups, which in turn can be reacted to form the onium group and the nucleophilic group.

In some embodiments, the arylene compound has a structure of formula (II):

Y₁—Ar—Y₂ (II) or a salt thereof, wherein:
Ar is or comprises an optionally substituted aromatic or optionally substituted arylene; and
each of Y₁ and Y₂ is, independently, a reactive group or a functional group configured to provide a reactive group (e.g., any described herein) upon reaction with a reactant.

In some embodiments, the arylene compound salt has a structure of formula (IIa):

Nu—Ar—X′ (IIa) or a salt thereof, wherein:
Nu is or comprises a nucleophilic group (e.g., any described herein);
Ar is or comprises an optionally substituted aromatic or optionally substituted arylene; and
X′ is or comprises a functional group configured to provide an onium group upon reaction with a reactant.

In some embodiments, Nu and X′ are provided in a para position.

Y₁ and Y₂ can include any reactive group (e.g., as described herein) or any functional group configured to provide a reactive group (e.g., any described herein) upon reaction with a reactant. In some embodiments, the functional group includes a nucleophilic group (e.g., any described herein) that further includes a protecting group (e.g., as described herein). Non-limiting examples of Y₁ and Y₂ include, independently, amino, halo, alkoxy, hydroxyl, thioalkyoxy, thiol, phosphinyl, and the like.

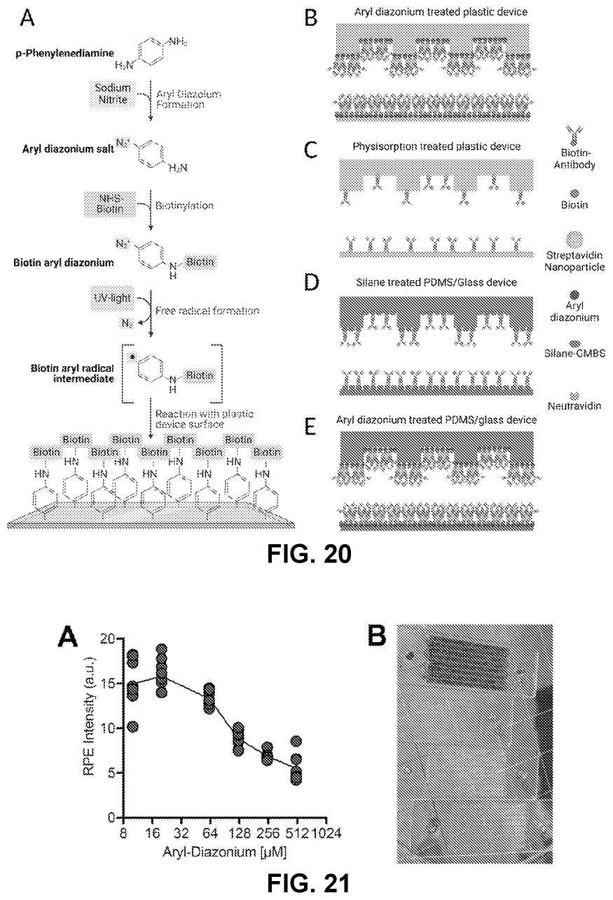

X′ can include any functional group configured to provide an onium group upon reaction with a reactant. Non-limiting examples of X′ include, independently, amino, halo, alkoxy, hydroxyl, thioalkyoxy, thiol, phosphinyl, and the like. Any of these X′ groups can be reacted (e.g., with a reactant provided herein) to provide an onium group (e.g., ammonium, a diazonium, a halonium, oxonium, phosphonium cation, or sulfonium, as described herein). FIG. 20 provides a schematic of a non-limiting method to produce and use an aryl onium salt.

The aryl-onium salt includes an onium group, which can be released, thereby forming an aryl compound having a free radical. Release can include exposing the onium group to radiation (e.g., ultraviolet radiation), heat, or electric field. Furthermore, release of the onium group can occur in the presence of the surface of the microfluidic device, thereby facilitating a reaction between the free radical and the surface.

FIG. 1F is schematic of a non-limiting method for using an aryl onium salt to provide a functionalized surface. As can be seen, the aryl onium salt 160 can include an arylene moiety (Ar), an onium group (X), and a nucleophilic group (Nu). Examples of arylene moieties, onium groups, and nucleophilic groups can include any described herein. In some embodiments, the onium group can be released 171 to provide an aryl radical 161, which in turn can be exposed 172 to a surface 165 to provide an attached arylene moiety (Ar). The remaining nucleophilic group (Nu) can be further reacted to attach a member of a binding pair, linker, a particle, or a capture agent.

Alternatively, the functionalized surface can include the use of a member of a binding pair. This member can be attached to the aryl onium salt, in which reactions can be conducted in solution or at the surface. In some embodiments, the method can include reacting 173 the aryl onium salt 160 to provide an aryl conjugate 162 having first member of a binding pair 162a. Such reactions can include the use of a reagent having the first member and an electrophilic group (e.g., an alkenyl group, an alkynyl group, a carbonyl group, an ester group, an imido group, an epoxide group, an amido group, a carbamido group, a cation, etc.), such that the electrophilic group can react with Nu to form a covalent bond.

Accordingly, in some embodiments, methods of functionalizing a surface can further include reacting a nucleophilic group of the aryl-onium salt with a first member of a first binding pair to form an aryl conjugate. In some embodiments, the aryl conjugate can include the arylene moiety disposed between the onium group and the first member of the binding pair.

As further seen in FIG. 1F, the onium group from the aryl conjugate 162 can be released 174, thereby forming an aryl conjugate-radical (163). This radical can be exposed to a surface 165 to provide an attached arylene moiety (Ar) having the first member 162a. The remaining first member 162a can be further reacted to attach a member of a binding pair, linker, a particle, or a capture agent.

The nucleophilic group (Nu) of the aryl onium salt can be reacted at any time. For instance, the nucleophilic group (Nu) on the aryl onium salt 160 can be reacted 173. Alternatively, the aryl onium salt can be in the form of a free radical. For instance, the nucleophilic group (Nu) on the aryl radical 161 can be reacted. Furthermore, surface-based reactions can be performed. For instance, the nucleophilic group (Nu) attached to the surface 165 can be reacted 177.

Accordingly, the method of functionalizing a surface can further include: reacting the nucleophilic group of the functionalized surface with a first member of a first binding pair. In some embodiments, the method can include: providing a particle comprising a second member of the first binding pair, wherein the first and second members bind together to form a bond. In other embodiments, the method can include: providing a linker comprising a second member of the first binding pair, wherein the first and second members bind together to form a bond.

In some embodiments, one or more capture agents can be attached to the particle. In some embodiments, a linker is disposed between at least one of the one or more capture agents and the particle.

In some embodiments, a second binding pair is disposed between the particle and the linker (e.g. a flexible linker). For example, a first member of the second binding pair can be provided on a surface of the particle, and a second member of the second binding pair can be attached to a flexible linker. The first and second members can form a bond, thereby providing a flexible linker extending from the particle. In some embodiments, a capture agent can be attached to the flexible linker. Such an attachment can include a covalent bond or a further binding pair. For instance, a third binding pair can be employed, in which a first member of the third binding pair is provided at an end of the flexible linker, and the second member of the third binding pair is directly or indirectly attached to the capture agent.

### Capture Agents and Targets

The devices, systems, and methods herein can employ one or more capture agents. In some embodiments, the capture agent is configured to interact with a surface of a virus in an intact form, a surface of a target cell, or a surface of a target vesicle (e.g., a target extracellular vesicle).

Non-limiting examples of capture agents include a protein, e.g., angiotensin-converting enzyme 2 (ACE2), a mutant form thereof, or a recombinant form thereof; an antibody, e.g., an antibody that binds a spike protein of the virus or a receptor binding domain (RBD) of the virus, or an antibody that binds to CD3, CD4, CD8, CD9, CD11b, CD14, CD16, CD19, CD20, CD31, CD45, CD63, CD66, CD66b, CD81, HLA-DR, TSG-101, epithelial cell adhesion molecule (EpCAM), epidermal growth factor receptor (EGFR), human epidermal growth factor receptor 2 (HER2), podoplanin, barrier-to-autointegration factor (BAF), platelet-derived growth factor receptor (PDGF), or ephrin receptor A2 (EphA2); an aptamer, e.g., an aptamer that binds a spike protein of the virus; lectin; heparin; a glycoprotein; a deoxyribonucleic (DNA) fragment, and the like.

Yet other non-limiting examples of capture agents include C—C chemokine receptor type 5 (CCR5), a mutant form thereof, or a recombinant form thereof (e.g., to bind human immunodeficiency virus (HIV)); cluster of differentiation 4 (CD4), a mutant form thereof, or a recombinant form thereof (e.g., to bind HIV); neutralizing antibody, KZ52, a mutant form thereof, or a recombinant form thereof (e.g., to bind ebolavirus (EBV)); laminin-5, a mutant form thereof, or a recombinant form thereof (e.g., to bind herpesvirus (HPV)); heparin sulfate proteoglycan, a mutant form thereof, or a recombinant form thereof (e.g., to bind HPV); cluster of differentiation 46 (CD46), a mutant form thereof, or a recombinant form thereof (e.g., to bind cytomegalovirus); and complement receptor type 2 (CR2), a mutant form thereof, or a recombinant form thereof (e.g., to bind EBV).

Capture agents can be directly or indirectly attached to an inner wall surface of a microchannel. Furthermore, a plurality of capture agents can be provided in same channel, in which each capture agent is same or different. In other embodiments, different capture agents can be provided in different channels. Each device can include one or more microchannels. When a plurality of devices is employed, each channel in each device can have same or different capture agents, and each device in a system can have same or different capture agents.

Capture agents can be selected based on the desired type of target to be captured. Non-limiting examples of targets include a virus, e.g., such as a coronavirus, an ebolavirus, an influenza virus, a hepatitis virus, a lentivirus (e.g., human immunodeficiency virus), a herpesvirus (e.g., Epstein-Barr virus), a cytomegalovirus, or an oncovirus (e.g., a retrovirus, a herpesvirus, a papillomavirus, a polyomavirus, a hepatitis virus, and the like); a cell, such as an immune cell (e.g., a T cell, a B cell, or an innate immune cell), an epithelial cell, an endothelial cell, or a neural cell; a vesicle, such as an extracellular vesicle, a vesicle from an immune cell (e.g., a T cell, a B cell, or an innate immune cell), a vesicle from an epithelial cell, a vesicle from an endothelial cell, or a vesicle from a neural cell; a vesicle from a damaged cell; or a combination of any of these. In some embodiments, the target is a virus in an intact form. In other embodiments, the target is a viral particle. Yet other examples of targets include living cells or microvesicles, e.g., leucocytes, CD4+ T-cells, fetal cells in maternal blood, or circulating tumor cells (CTC).

The target can include any vesicle present in circulation, which in turn can be characterized to identify the source of the vesicle (e.g., a specific cell type or a specific organ) and/or to determine the extent of organ-specific damage. For instance, damaged cells or cells in distress can release vesicles, e.g., for cardiac damage, vesicles can be released from activated endothelial cells; for liver, vesicles can be released from hepatocytes and/or endothelial cells; for lung, vesicles can be released from epithelial cells; for brain, vesicles can be released from neural cells, etc.

Cargoes within targets can be analyzed. Non-limiting examples of cargoes include deoxyribonucleic acid (DNA), ribonucleic acid (RNA, including, e.g., mRNA), proteins, lipids, and cytokines. Any of these cargoes can serve as a marker.

Extracellular vesicles (EVs) can be detected using the devices, systems, and methods herein. EVs can include exomeres (e.g., having a size of less than or equal to about 50 nm), supermeres (e.g., having a size of less than or equal to about 50 nm), exosomes (e.g., having a size from about 40 to 200 nm), large EVs or microvesicles (e.g., having a size from about 200 to 1000 nm), oncosomes (e.g., having a size greater than about 1000 nm), or a combination of any of these. EVs can be characterized by having one or more surface markers, which in turn can be captured by a capture agent that binds to that surface marker. Non-limiting examples of surface markers include CD3, CD4, CD8, CD9, CD11b, CD19, CD20, CD31, CD37, CD41, CD44, CD45, CD56, CD62p, CD63, CD66b, CD73, CD81, CD82, TSG-101, Alix, flotillin-1, clathrin, Hsp60, Hsp70, Hsp90, syntenin-1, Rab27a, MMP-9, natural killer group 2 member D (NKG2D/CD314), HLA-DR, extracellular matrix metalloproteinase inducer (EMMPRIN/CD147), epithelial cell adhesion molecule (EpCAM), or epidermal growth factor receptor (EGFR), and the like.

EVs can be derived from any cell type. Different types of target EVs can captured and isolated depending on the clinical application. For instance, isolation and capture of tumor-derived EVs can be used to identify the presence of tumor cells producing the tumor-derived EVs in a biological sample. As an example, target EVs can represent EVs produced by tumor cells that are associated with different pathological conditions, such as brain, pancreatic, prostate, lung, breast, bladder, liver, and head and neck cancers. Target EVs can also be derived from cells associated with the tumor or tumor microenvironment, such as macrophages, neutrophils, immune cells, and T-cells. Isolation of these cell-specific EVs can help in the identification of patients that will respond to specific treatments, with a direct interest in immunotherapy. Further, these EVs can help to identify patients that are responding to the treatment already administered. Other non-cancer disease states (or injuries) would include cardiac events, stroke, neurological conditions (Parkinson's, Huntington's, Alzheimer's, Schizophrenia, Traumatic Brain Injury) as well as monitoring mental health and treatment response.

In other instances, target EVs can represent EVs produced by other types of cells of interest. For example, EVs released from putative donor organs can be used to monitor the ‘fitness’ of the organs for transplant. All biological cells release EVs, and as such, they can represent a biomarker for overall organ health and state. Examples include cardiac, kidney, and liver EVs. Immune response and allergic reactions could also be monitored through EV release from specific cells, while their production in animal products (e.g., cow's milk) help to identify both fertility states as well as a means for quality control of the food source.

### Samples

The devices, systems, and methods herein can be employed with a sample (e.g. a test sample) to capture desired targets. In some embodiments, the sample is or includes a diluted sample, a stabilized sample, a preserved sample, or a combination thereof.

Any clinical biofluid or specimen matrix may be employed. In some embodiments, the specimen is mixed to a solvent (e.g., water, a buffer, an aqueous solvent, an organic solvent, or a combination thereof) to provide a solution or a suspension. In other embodiments, the biofluid or specimen can be diluted and/or stabilized. Non-limiting examples of samples can include blood, plasma, serum, stool, saliva, urine, sputum, or waste water.

The emergence of cutting-edge clinical technologies has created a significant demand for the biostabilization of blood and other bodily fluids during transportation and storage. Although strategies to preserve purified components of whole blood have been relatively successful, whole blood stabilization remains elusive. Lack of a preservation method can hinder the wide-spread dissemination of blood based analytical and diagnostic technologies, which rely on viable cells with intact RNA. Hemolysis, platelet activation, cytokine and oxidative bursts, and neutrophil extracellular trap formation can occur within hours of blood collection. This deterioration can interfere with microfluidic applications and enrichment technologies, such as cell sorting. Accordingly, samples for use with the microfluidic devices and systems herein may be stabilized.

Stabilization of samples can include any combination of strategies. In one approach, a combination of storage temperatures, platelet inhibitor cocktails, preservatives, and chemical additives aimed at biochemical and biophysical stabilization during transport can be used. In another approach, a preservation formulation in combination with a caspase inhibitor and/or a platelet inhibitor can be used. Other approaches can employ a Ficoll polymer (e.g., Ficoll 70). Preservatives can be employed, such as a buffer (e.g., HEPES), adenine, mannitol, acetyl-L-cysteine, dextrose, salt (e.g., NaCl), F68 (e.g., a triblock copolymer of the form polyethylene oxide-polypropylene oxide-polyethylene oxide (PEO—PPO—PEO)), lactobionate, trisodium citrate, citric acid, Trolox (6-hydroxy-2,5,7,8-tetramethylchroman-2-carboxylic acid), ascorbic acid, or a combination thereof. Cryoprotectants and ice nucleating agents can be employed, such as, e.g., dimethylsulfoxide, ethylene glycol, propylene glycol, propane-1,2-diol, 3-O-methyl-glucose, glycerol, sucrose, trehalose, stachyose, raffinose, silver iodide, a polymer, a protein, a carbohydrate, a phospholipid, a hydrogel particle, or a combination thereof. Yet other approaches can employ an anti-coagulant. Other approaches and strategies are described in U.S. Pat. Pub. Nos. 2020/0305415, 2021/0007348, and 2022/0104482, each of which is incorporated herein by reference in its entirety.

### Uses

The devices and systems herein can be employed in a method that would benefit from specific capture of desired targets. Such methods can be used to treat, diagnose, monitor, or otherwise manage a disease, a syndrome, a condition, or a symptom from a disease or syndrome, such as in a viral infection (e.g., a SARS-COV-2 infection, an Epstein Barr infection, an Ebola infection, an HIV infection, etc.); conditions related to infections (e.g., post-acute sequelae SARS-COV-2 infection (PASC) or multisystem inflammatory syndrome in children (MISC-C), and the like); cancer treatment, such as for treatment selection and/or for monitoring in immunotherapy, e.g., immune-related adverse events (irAE); cardiovascular disease, such as for monitoring or managing acute cardiac events; and autoimmune disease, such as monitoring of type 1 diabetes biomarkers for pancreatic organoids. Such methods can also include those for continued monitoring based on a certain patient population, e.g., stool monitoring for pediatric patients, immunotherapy monitoring for cancer patients, etc.

In particular embodiments, the present document encompasses methods of isolating a target in a sample. In one aspect, the target can be a virus. A non-limiting method can include: flowing a sample comprising the virus through a microchannel (e.g., any described herein); capturing the virus in an intact form using a capture agent in the microchannel; lysing the intact form of the virus in the microchannel, thereby providing a lysate; and analyzing the lysate to determine the presence of one or more markers of the virus.

In some embodiments, the microchannel includes a inner wall surface, at least one groove is defined in the inner wall surface, and a capture agent configured to interact with a surface of the virus. In some embodiments, the capture agent can be attached to the inner wall surface and/or the groove. In other embodiments, the microchannel includes a first inner wall surface and a second inner wall surface that is different than the first inner wall surface, in which a groove is defined in the first inner wall surface and in which the capture agent is disposed on the second inner wall surface. In yet other embodiments, the groove and the capture agent is disposed on the same first inner wall surface.

In some embodiments, the method can include collecting the sample after flowing through the microchannel, thereby providing a collected sample. If the captured targets remain attached within the microchannel, then the collected sample can include a target-depleted sample. If the captured targets are lysed or released from the microchannel, then the collected sample can include a target-enriched sample. In some embodiments, the collected sample can include one or more target cells or target vesicles. In other embodiments, the collected sample can include a lysate.

The target-enriched or target-depleted sample can be collected and analyzed. Any desired markers can be analyzed. For instance, the one or more markers can include a nucleic acid (e.g., RNA, DNA, or both), and analysis can include amplifying or sequencing the one or more markers. Amplification can include the use of an isothermal amplification reaction. Optionally, one or more detection reagents can be delivered to the microchannel. Analysis can be performed on-chip or off-chip.

Analysis can include other determined characteristics. In some embodiments, analysis can include determining a concentration of the virus captured by the capture agent in the microchannel. Such determinations can include one or more optical measurements, amplification reactions, sequencing, resistive pulse sensing, or particle analysis to measure a concentration of viral particles captured by the capture agent. For instance, a method can include determining viral load in a sample. A non-limiting method can include: flowing the sample through a first microchannel comprising a first inner wall surface, wherein the first inner wall surface comprises a first capture agent configured to interact with a surface of a viral particle; capturing the viral particle in an intact form using the first capture agent in the first microchannel; and measuring a concentration of viral particle captured in the first microchannel.

In another aspect, the target can include a plurality of targets, in which a first target is a virus and in which a second target is a cell (e.g., a B cell, an innate immune cell (e.g., a neutrophil, a macrophage, etc.), an epithelial cell, an endothelial cell, or a neural cell) or a vesicle (e.g., an extracellular vesicle or others described herein).

A non-limiting method can include: flowing the sample comprising a virus through a first microchannel comprising a first inner wall surface, wherein the first inner wall surface comprises a first capture agent configured to interact with a surface of the virus; capturing the virus in an intact form using the first capture agent in the first microchannel; collecting the sample after flowing through the first microchannel, thereby providing a collected sample comprising one or more target cells or target vesicles; flowing the collected sample through a second microchannel comprising a second inner wall surface, wherein the second inner wall surface comprises a second capture agent configured to interact with a surface of the target cell or a surface of the target vesicle; and capturing the target cell or the target vesicle in an intact form using the second capture agent in the second microchannel.

In some embodiments, the method can include collecting the sample after flowing through the first microchannel and then through the second microchannel, thereby providing a first collected sample and a second collected sample, respectively. In some embodiments, the first collected sample can include one or more target viruses, and the second collected sample can include one or more target cells or target vesicles. Collected sample(s) can be analyzed for markers, such as viral markers (e.g., viral nucleic acid), immune markers, and the like. Optionally, one or more detection reagents can be delivered to the microchannel. Again, analysis can be performed on-chip or off-chip.

### EXAMPLES

### Example 1: Microfluidic SARS-CoV-2 Platforms

Novel coronavirus (SARS-Coronavirus-2: SARS-COV-2) is thought to be a systemic disease, impacting multiple organs with major syndromic complexity. As the virus continues to spread internationally, there is a need for both diagnosis at the earliest stages of infection, as well as methods to better stratify COVID-19 patients at the time of presentation. As yet, most virus detection assays rely on the detection of viral RNA obtained through a nasal swab. Recent analysis of nasal pharyngeal PCR tests reports a 100% probability of receiving a false negative result one day post-exposure to a COVID+ individual. Waiting four days post-exposure, the probability of a false negative rate is still alarmingly high at 67%. Even eight days post-exposure, the likelihood of receiving a false negative is 20%. Towards providing reliable, earlier testing for SARS-COV-2 detection, the present document describes a microfluidic-based technology that can capture and isolate even rare cell-specific exosomes with high sensitivity and specificity. Having a herringbone (HB) structure, the platform is referred herein as the HB-Chip.

SARS-COV-2 viral levels are thought to be extremely low in whole blood, with early data reporting detection rates as low as 1% in blood. The HB-Chip can be used to find even the rarest events in blood. Furthermore, other sample types could exhibit higher detection rates, depending on the route or progress of viral infection. The present document also describes the use of the microfluidic platform for alternative sample sources, such as saliva and stool. Regardless of biospecimen source, the microfluidic platform can be optimized to provide a highly sensitive (95% sensitivity) and specific (95% specificity) test for SARS-COV-2 diagnosis that enables detection at the earliest time of infection.

Microfluidics is an enabling technology for rare particle isolation, including particles such as rare circulating cells and exosomes from patient blood. In particular, the microfluidic platform can be designed to preferentially capture the desired targets. Due to the heterogeneity inherent in extracellular vesicles (EVs), the approach for EV isolation is not dependent upon size, nor should it rely on a generic marker for capture. Rather, the capture techniques described herein employ capture agents that bind to desired targets to enrich certain cells, particles, or cell-specific EVs.

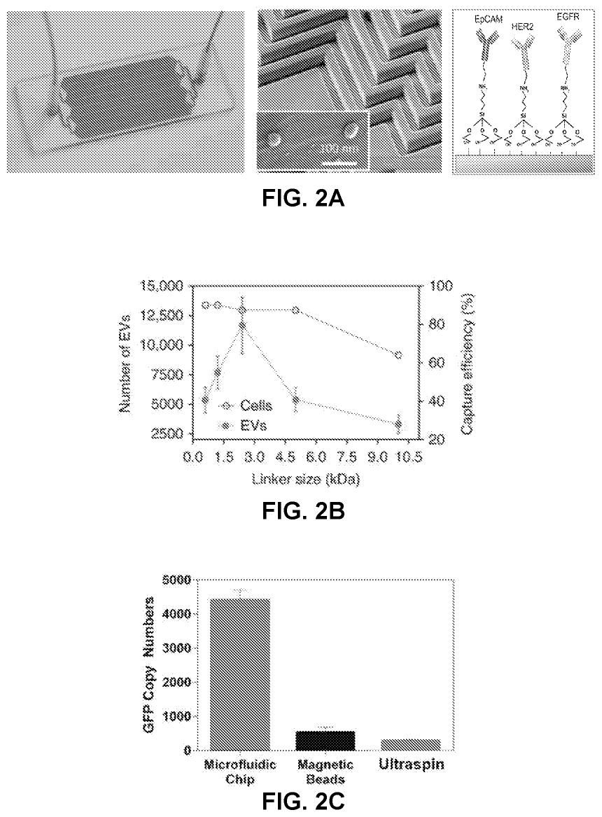

In addition to use of capture agent, the HB-Chip employs certain structural features to optimize interactions between the fluid sample and the capture agent. FIG. 2A shows a non-limiting HB-Chip. Briefly, the geometry of the chip induces chaotic mixing of the fluid by disrupting the streamlines and maximizing collisions between particles and the walls of the device. By providing capture agents (e.g., antibodies, proteins, or other capture agents described herein) near the walls, the geometry facilitates increased interactions between the capture agent and the desired target. To further maximize capture of rare particles such as EVs, the chip can be modified to reduce steric hinderance effects that limit small particles from interacting with the chip surface (where the capture agents are located). As seen in FIG. 2B, the linker size is one parameter that can be optimized to provide desired capture efficiency. In particular, >100 fold enrichment of tumor EVs was achieved (FIG. 2C), which was about 25 times greater than other microfluidic approaches with the same goal.

The microfluidic platform can be optimized to isolate the rarest of events in complex fluids. For instance, viral detection may be limited by the number of virus present in blood. Of note, blood is thought to not have much virus. Yet, while circulating cancer cells may also be considered rare events, such cells were effectively captured when interrogating samples with microfluidic devices. Upon capture of rare tumor exosomes, nucleic acid (e.g., RNA and DNA) from nanoscale vesicles can be analyzed to inform on the molecular content of the tumor.

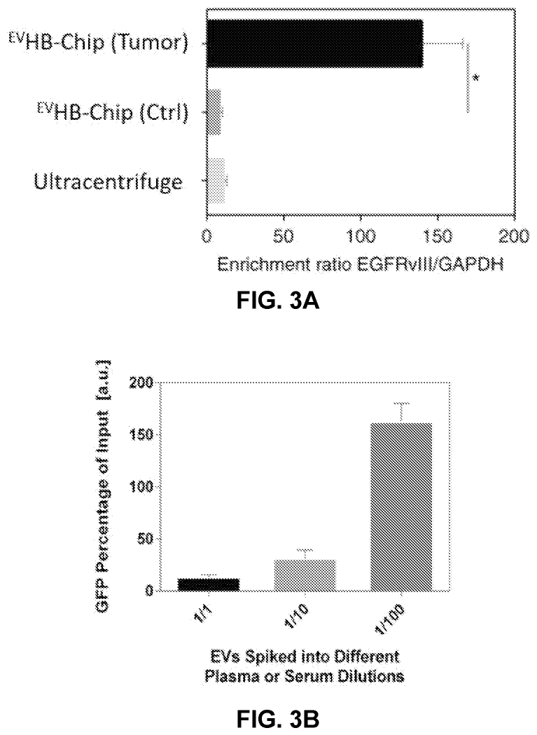

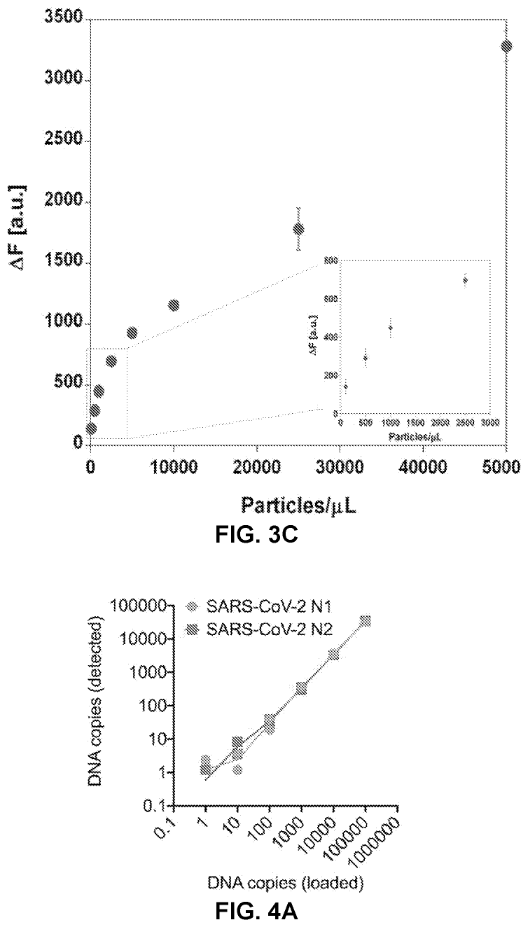

As more is understood about SARS-COV-2, data are suggesting that viral levels may be higher in blood than previously thought. As such, the microfluidic platforms herein (e.g., devices, systems, channels, etc.) can be adapted for SARS-COV-2 isolation. Further, the performance of this technology only improves as the target analyte becomes increasingly rarer in plasma (see, e.g., FIG. 3B). Due to the exquisite sensitivity of the HB-Chip, one cell in a billion or 1 EV in 1 μL of plasma can be identified (FIG. 3C).

The microfluidic platform exhibits significant advantages, including for example and without limitation: (1) processing of minimal amounts of sample (e.g., as low as 100 μL), which can be critical when COVID-19 specimens are in high demand; (2) a limit of detection of 100 EVs in 100 μL of plasma (or 1 EV per μL), allowing for isolation of very rare populations of particles, including viral particles, cells, or cell-specific EVs; and (3) capture of intact particles, which allow for high quality RNA and protein analysis and better assessment of infectivity from RNA measurements of biofluids. In addition, capture of intact particles can allow for counting or measuring the concentration of particles within a sample, which can be used to test whether patients are infectious depending on the viral load detected. Capture of intact particles and subsequent lysis of such particles can allow for efficient extraction of markers (e.g., nucleic acids, proteins, peptides, etc.) from the particle, as compared to bulk extraction without using chip-based capture. Thus, as described herein and in the following Examples, the microfluidic platforms herein can allow for effective isolation and capture of virus and for rapid diagnosis of viral infection.

### Example 2: On-Chip Capture of Plasma Spiked with SARS-CoV-2 Pseudovirus

Pseudovirus particles were employed assay optimization. In particular, the tested SARS-COV-2 pseudoviral particles express the spike protein. Briefly, the SARS-COV-2 pseudovirus is generated by transfecting HEK 293T cells with a psPAX2 lentiviral packaging vector, pSin-RFP (RFP-expressing vector), and a plasmid encoding SARS-COV-2 Spike protein with a signaling peptide to package the spike protein on the surface of the pseudovirus. These pseudoviral particles enter ACE2-expressing cells via the spike protein and induce expression of RFP. The particles are not able to propagate, making them ideal candidates for initial characterization of viral capture by the HB-Chip.

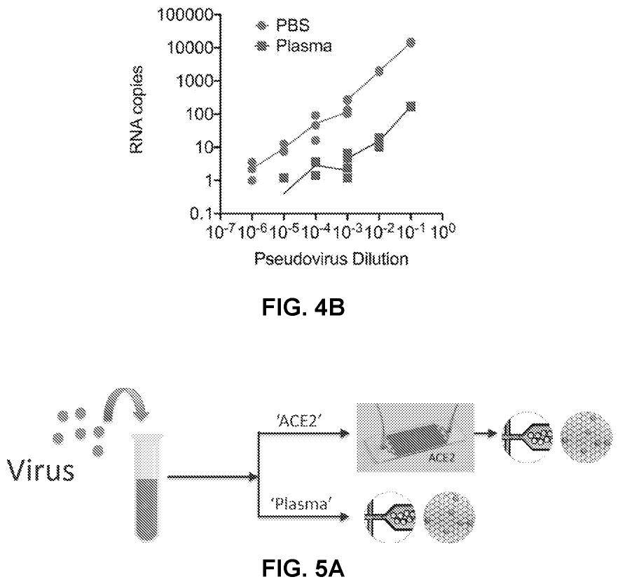

Knowing that we wanted to push the limits of detection for SARS-COV-2 capture, tests were conducted to verify that the downstream RNA assay could detect as little as one viral RNA copy. Using the FDA-approved SARS-COV-2 probes (Integrated DNA Technologies, Inc., Coralville, IA) and digital droplet PCR (ddPCR), the downstream quantification of SARS-Cov-2 RNA was benchmarked. Using simple bulk analysis of viral DNA plasmids, the LOD for the quantification assay was established (FIG. 4A). In particular, one viral copy of both the N1 and N2 probes was detected. Next, the effect of the biofluid on ddPCR detection sensitivity was characterized. Here, pseudovirus was spiked into plasma and PBS in a 10× dilution series. The LoD for plasma capture is a 1×10⁻³ dilution, but pseudovirus is detectable down to 1×10⁻⁶ in PBS (FIG. 4B). Therefore, without the benefit of microfluidic isolation, plasma drastically decreases the ability of ddPCR to detect viral particles from bulk extraction in plasma.

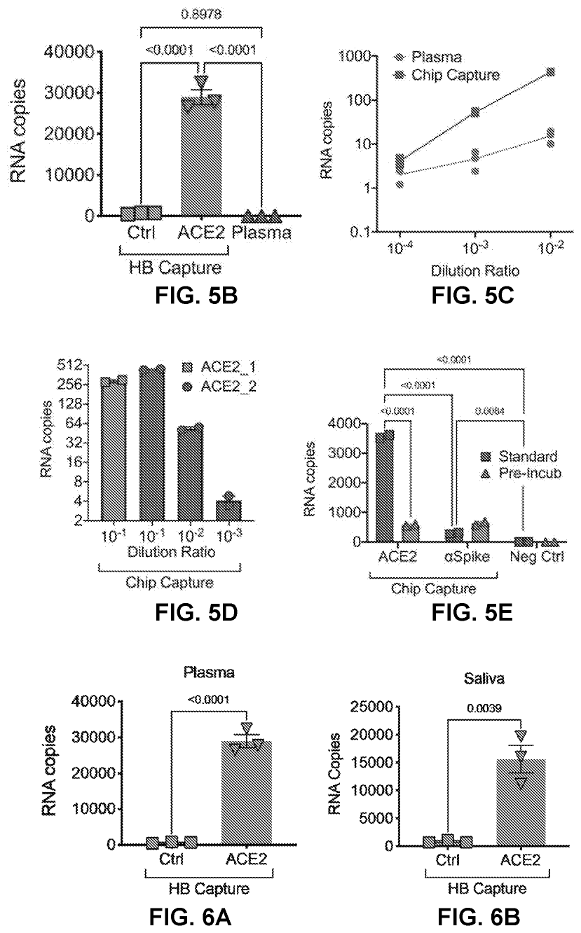

Based on these data, the HB-Chip for viral isolation was developed by using an ACE2 protein attached to the inner surface of the chip for capture. A set volume of plasma was spiked with pseudovirus, again using a 10× dilution series. For the “ACE2” or “Chip Capture,” an aliquot of the same prepared spiked plasma was flowed through the chip (FIG. 5A). For the “Plasma” analysis, the sample was analyzed by a bulk ddPCR assay. Following capture, the virus was lysed and RNA extracted followed by analysis using the ddPCR assay. As seen in FIG. 5C, the HB-Chip was able to detect SARS-COV-2 with a much lower LOD than the traditional bulk analysis. These early experiments showed a 25-fold ability to detect pseudovirus in plasma, as compared to isolation in solution (FIG. 5C).

To isolate the virus from any biofluid, a capture agent was used. Such agents can include an antibody, protein, or aptamer that binds to the surface of an intact virus. Different capture agents were tested. Different sources of ACE2 proteins for viral capture were tested, and no difference in binding was observed (FIG. 5D). While the standard protocol calls for attaching the antibody or another capture agent to the chip surface prior to loading the sample, another approach can include binding of ACE2 to viral particles in solution, as compared to the standard surface-based capture protocol. Both approaches were tested (FIG. 5E), and the standard capture strategy outperformed ‘in solution labeling’ of SARS-COV-2. The standard method had a 6-fold increase in signal, as compared to pre-incubation (FIG. 5E). Lastly, capture of SARS-COV-2 was compared by using ACE2 protein or an antibody against the spike protein (FIG. 5E, data labeled “ACE2” versus “αSpike”).

### Example 3: Benchmarking the Use of HB-Chip in Plasma and Saliva

Further tests were performed to benchmark the efficiency of chip-based capture using the HB-Chip, as compared to bulk plasma analysis that did not employ the HB-Chip. In three independent experiments, the HB-Chip was able to show a drastically higher efficiency at identifying viral RNA in plasma from intact viral particles, as compared to bulk plasma (FIG. 5B and FIG. 6A). Additionally, compared to a non-specific control chip, very low amounts of non-specific binding of pseudovirus was observed, as compared to the ACE2 capture chip.

The specificity of the capture strategy was tested in saliva (FIG. 6B). Again, pseudovirus was spiked into saliva from a healthy donor at the 1×10⁻³ dilution used. In a run of three independent chips, the HB-Chip showed a high efficiency at identifying viral RNA in saliva from intact viral particles. Additionally, compared to a non-specific control chip, very low amounts of non-specific binding of pseudovirus was observed, as compared to the ACE2 capture chip. Overall, these data suggest that the HB-Chip can isolate virus from complex biofluids and warrants further development as a highly sensitive point-of-care assay for detection of SARS-COV-2, other future viral outbreaks, or other rare circulating particles suspected to be present in a biofluid.

### Example 4: Capture of SARS-CoV-2 Variants and Other Viruses

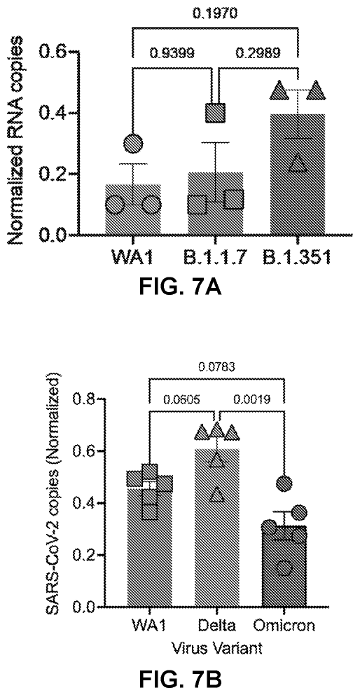

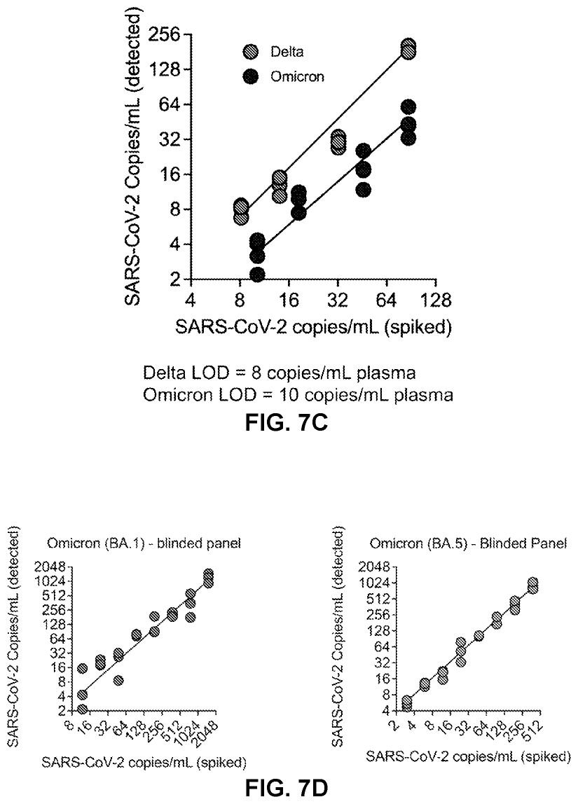

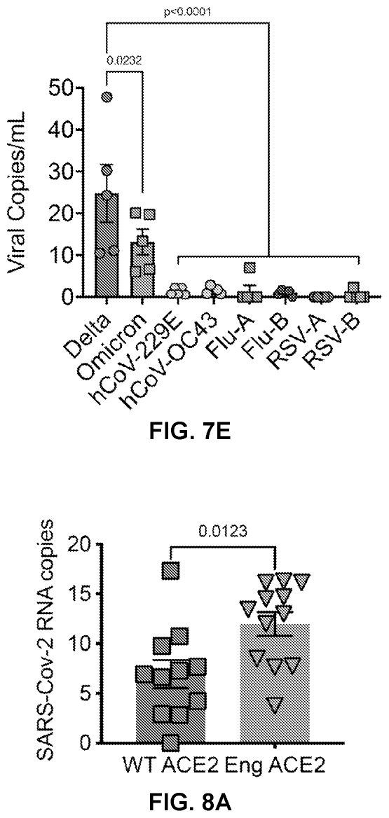

The assay can be developed to capture known SARS-COV-2 sequences, as well as variants that may include one or more mutations for such sequences. FIG. 7A-7E provides detection of various variants for SARS-COV-2 using the chip-based assays described herein.

Further analysis can include testing for cross-reactivity with other common or related viruses (e.g., MERS-COV or SARS-COV-1). Cross-reactivity for assay primers and probes can be evaluated through in silico analysis. For a selected sample type, flora and other viral pathogens specific to the matrix (saliva, stool, etc.) can be identified and tested. Probes to target SARS-COV-2 can be analyzed to ensure they possess less than 80% homology (e.g., using standard homology modeling approaches) to probes that target common or related viruses. FIG. 7E provides cross-reactivity results of the chip-based assay for SARS-COV-2 variants and six other viruses: human coronavirus 229E (hCoV-229E), human coronavirus OC43 (hCoV-OC430, respiratory syncytial virus A and B (RSV A and B), and influenza virus A and B (Flu A and B). Viruses were spiked into plasma, run with the HB-Chip, and tested for the virus pushed through the device.

In addition, the assays herein can be designed for multi-analyte capture (e.g., using a respiratory panel). For instance, when a patient is tested for COVID-19 symptoms, they can typically be simultaneously tested for other virus infections (e.g., respiratory syncytial virus (RSV), influenza virus, and the like). The optimization work for processing saliva, plasma, stool, and other matrices in an HB-Chip for SARS-COV-2 capture can be transferrable for a multi-analyte capture platform. Furthermore, marker detection can include an amplification reaction that is optimized for multiplexed detection of various markers. Capture agents can be selected based on the other desired targets in the multi-analyte capture platform.

### Example 5: Optimization of Viral Capture in the HB-Chip

The assay can be optimized for cost efficiency, maximum detection of viral RNA from samples, or both. Optimization approaches can include testing the limit of detection, assessing capture strategy, diluting of biofluid(s) or biospecimen(s), and/or determining the effect of viscoelastic force on capture in the chip.

The capture of viral particles with the HB-Chip can be assessed with pseudoviral particles. Such particles can be spiked into plasma, saliva, stool, and other specimens from 0 to 1×10⁶ particles/mL in a 2-fold dilution series. Pseudoviral specimens can be flowed through the HB-Chip to establish the sensitivity and limit of detection (LOD) of devices for all fluids. Binding strategies can be compared through two readouts: (1) palm-GFP tagged pseudoviral particles captured on chip or (2) RFP RNA amount isolated from the chips. To determine the fluorescent intensity of bound pseudovirus, sections of the chip can be imaged and compared to negative controls. For these assays, negative controls can include either: (1) a sample alone (e.g., plasma/saliva/stool alone) or (2) a non-specific IgG chip with pseudovirus-spiked sample (e.g., pseudovirus-spiked plasma/saliva/stool). RNA can be extracted from the chips (e.g., using the Direct-zol kit from Zymo Research Corp., Irvine, CA), and cDNA can be created using cDNA synthesis protocols (e.g., using the PrimeScript first strand cDNA Synthesis kit from Takara Bio USA, Inc., San Jose, CA). The number of RFP copies present can be determined by ddPCR to assess how many pseudoviral particles containing RFP RNA were bound to the chip. Pseudoviral particles can be titrated down, and RFP RNA copies can be correlated to the number of viral particles added to the chip, thereby determining percentage of bound particles and sensitivity of the assay for detecting low numbers of virus. To additionally assess the specificity of the HB-Chip to capture SARS-CoV-2 spike protein, VSV-G pseudotyped lentiviral particles containing GFP can be used as a negative control to determine non-specific binding to the HB-Chip.

Capture efficiency may be influenced by dilution of the sample and the effect of viscoelastic forces that may be present within the HB-Chip. Previous studies from our group have shown that dilution of biofluids can increase the ability to capture rare molecules. In particular, the HB-Chip can be optimized for isolation of rare cells and vesicles in complex biofluids like blood and plasma with high viscosity and, therefore, high viscoelastic forces. Because the volume of samples can be often limited, the effect of dilution on virus isolation from biofluids can be assessed. Ficoll® (a polysaccharide) has a similar viscosity to blood and plasma and can serve as an attractive material for dilution of samples, while increasing likelihood of binding in the HB-Chip. Polymers with similar physiochemical properties, such as dextran and polyethylene glycol, e.g., dextran 40 kDa polymers, can also be used. Each biofluid can be tested in an undiluted form or a diluted form (e.g., 1:1, 1:2, and 1:4 with Ficoll (e.g., Ficoll® 70)). The biofluid can be spiked with pseudovirus at a determined LOD to assess the effect of dilution on the ability to capture rare viral particles.

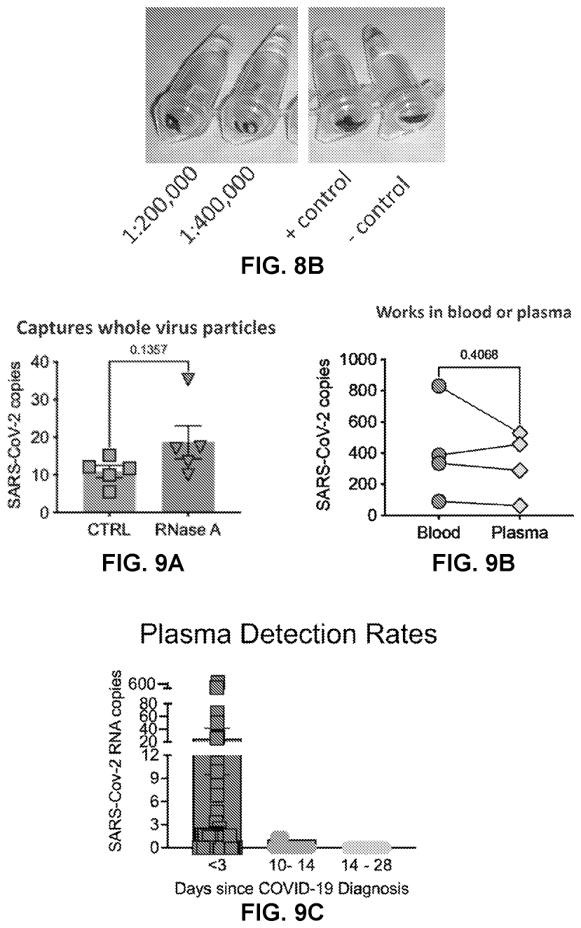

Other capture strategies can be implemented. In one instance, the capture strategy can use ACE2 capture, which has shown a demonstrated ability to capture SARS-COV-2. Other strategies include the use of mutant ACE2 forms, engineered ACE2 forms (see, e.g., FIG. 8A) as well as aptamers. In one non-limiting embodiment, the engineered ACE2 can include or is a polypeptide sequence having at least 90% sequence identity to SEQ ID NO: 30 or a polypeptide sequence that is substantially identical to the sequence of SEQ ID NO: 30:

(SEQ ID NO: 30)
STIEEQAKTFLDFFDSQAEDLFYQSSLASWNYNTNITEENVQNMNNAGD
KWSAFLKEQSTLAQMYPLQEIQNLTVKLQLQALQQNGSSVLSEDKSKRL
NTILNTMSTIYSTGKVCNPDNPQECLLLEPGLNEIMANSLDYNERLWAW
ESWRSEVGKQLRPLYEEYVVLKNEMARANHYEDYGDYWRGDYEVNGVDG
YDYSRGQLIEDVEHTFEEIKPLYEHLHAYVRAKLMNAYPSYISPIGCLP
AHLLGDMWGRFWTNLYSLTVPFGQKPNIDVTDAMVDQAWDAQRIFKEAE
KFFVSVGLPNMTQGFWENSMLTDPGNVQKAVCLPTAWDLGKGDFRILMC
TKVTMDDFLTAHHEMGHIQYDMAYAAQPFLLRNGANEGFHEAVGEIMSL
SAATPKHLKSIGLLSPDFQEDNETEINFLLKQALTIVGTLPFTYMLEKW
RWMVFKGEIPKDQWMKKWWEMKREIVGVVEPVPHDETYCDPASLFHVSN
DYSFIRYYTRTLYQFQFQEALCQAAKHEGPLHKCDISNSTEAGQKLFNM
LRLGKSEPWTLALENVVGAKNMNVRPLLNYFEPLFTWLKDQNKNSFVGW
STDWSPYAD.

Other capture agents can be employed, such as those binding to certain regions of the spike(S) protein (e.g., the receptor binding domain (RBD) of the S protein). These molecules can serve as clinical decoy receptors. If such molecules provide higher binding affinity for the spike protein, then such capture agents can be used to capture pseudovirus in the HB-Chip. Capture chips can be tested for each molecule at a determined LOD of pseudovirus, and a cocktail of capture agents (e.g., three ACE2 and one aptamer) could also be tested. Further testing can include titrating down pseudovirus in a 2-fold dilution series below a determined LOD to compare differing capture strategies.

Limit of Detection (LOD) can be used to assess the HB-Chip. LOD analysis can be performed using inactivated SARS-COV-2 viral particles spiked into each patient biofluid. For testing, inactivated SARS-COV-2 may be employed. The early range of spike-in concentrations can be determined based on the range observed from pseudoviral LOD experiments outlined above. Starting at approximately 10-20 times higher than the LOD of pseudovirus, inactivated SARS-COV-2 in a 2-fold dilution series can be added to patient biofluid to determine LOD.

Optimization can include the use of preserved and stored samples. While nasopharyngeal swabs are a sensitive method of testing, they can present a risk to health care workers collecting the samples as well as increase the need to personal protective equipment use. If samples could be preserved and collected at home, it would increase the ability of chip-based tests to be used broadly in outbreaks of zoonotic coronaviruses. Preservation approaches can include those used for preserving RNA in complex biofluids. One approach can include the use of preservatives (e.g., as provided in RNA Complete BCT® from Streck Inc., La Vista, Nebraska; or ethylenediaminetetraacetic acid (EDTA)) for preserving pseudovirus spiked into healthy donor blood. Samples can be stored (e.g., at 4° C. or 25° C. for a period of one day, three days, five days, and seven days). Pseudovirus can be captured on-chip, and viral load can be measured (e.g., as described herein through ddPCR of RFP).

Viral load assays can be developed. In the infectious disease community, viral concentrations are typically reported by assays such as viral plaque assays, focus forming assays, endpoint dilution assays, or others. These assays indirectly report the viral concentration by their ability to either form plaques (e.g., as plaque forming units, PFU) or produce a cytopathic effect in a fraction of cells in a culture plate (e.g., as fifty percent tissue culture infective dose, TCID₅₀). The HB-Chip can be used to directly measure the concentration of physical viral particles captured on the device by optical measurements, amplification reactions, sequencing, resistive pulse sensing, downstream nanoparticle analysis, or other methodologies. This enables quantification of viral infection that is currently infeasible with existing methods of nucleic acid extraction and amplification. Importantly, the chip-based technology can differentiate between intact viral particles from circulating viral RNA. This could be one consideration to determine if a patient has cleared the infection and is likely no longer contagious. Possibly, a measure of infectivity can be determined using the assays described herein.

FIG. 7A-7D and FIG. 9A-9C shows non-limiting capabilities of the chip-based assay. As can be seen, the assay can be used across variants (FIG. 7A-7B) and works to capture whole viral particles (FIG. 9A). In particular, limit of detection (LOD) can provide a measure of the sensitivity of the assay (FIG. 7C-7D), and various sample matrices can be employed (FIG. 9B). Plasma detection rates can also be determined (FIG. 9C).

### Example 6: Point-of-Care Virus Detection

Digital droplet PCR (ddPCR) provides a sensitive methodology to detect nucleic acid. Yet, other methodologies can be implemented for point-of-care (POC) virus detection. In one instance, a loop-mediated isothermal amplification (LAMP) based assay can be employed. By using LAMP or reverse transcription loop-mediated isothermal amplification (RT-LAMP), multiple thermal cycles can be avoided, thereby simplifying integration of the detection module with the capture module provided by the HB-Chip. In this way, a sample can be analyzed on a unified device at POC.

To determine if the sensitivity of the assay translates from ddPCR to LAMP, RNA from patient samples can be tested using a LAMP assay targeting the nucleocapsid (N) gene and envelope (E) gene of SARS-COV-2 (e.g., such as the assay for WarmStart® LAMP Kit from New England BioLabs, Inc., Ipswich, MA). In one non-limiting instance, the assay can avoid the use of RNA extraction operations or kits. After capture, viral particles can be released and lysed (e.g., thermal lysing at about 95° C. or chemical lysing) on the chip in a monolithic device, thereby avoiding nucleic acid isolation. Reagents for LAMP or RT-LAMP reagents can be directly injected into the HB-Chip, and LAMP or RT-LAMP can be performed directly on the device. Once amplification has been performed, a colorimetric readout can be observed directly on the device. FIG. 8B shows results from a non-limiting LAMP assay.

### Example 7: Identifying Biofluids for COVID-19 Detection

Differing clinical samples (or biofluids) can provide differing levels of viral particles available for detection. Methodologies herein can include determining a biofluid useful for clinical validation. For instance, the sample can include plasma, saliva, stool, and other specimens. For clinical validation, differing samples can be obtained from symptomatic COVID positive (COVID+) patients, asymptomatic COVID+ patients, and confirmed COVID negative (COVID−) patients. For capture, the HB-Chip can be functionalized using a strategy that provides enrichment of intact viral particles (e.g., any strategy described herein). Table 1 provides detection for various samples using an HB-Chip. For this data set, tested samples included the following: COVID+ plasma (200 μL, n=12), stool (300 μL raw stool, n=4) and saliva (100 μL, n=2). For this analysis, a universal positive threshold was determined for the ddPCR assay and applied to all samples. The results in Table 1 provided a 42% detection rate in plasma samples, and further optimization strategies may be employed (e.g., any strategies described herein). Furthermore, this approach can be applied to other types of specimens (e.g., whole blood, interstitial fluid, nasopharyngeal samples, and the like).

TABLE 1
Detection of SARS-CoV-2 in COVID-19
Patients using the HB-Chip

| Sample Type | Positive | Negative | Detection [%] |
|---|---|---|---|
| Plasma (COVID+, n = 12) | 33 | 17 | 66% |
| Plasma (COVID−, n = 3) | 0 | 0 | 0% |
| Stool (COVID+, n = 4) | 7 | 3 | 70% |
| Stool (COVID−, n = 4) | 0 | 4 | 0% |
| Saliva (COVID+, n = 2) | 6 | 4 | 60% |
| Saliva (COVID−, n = 6) | 0 | 6 | 0% |

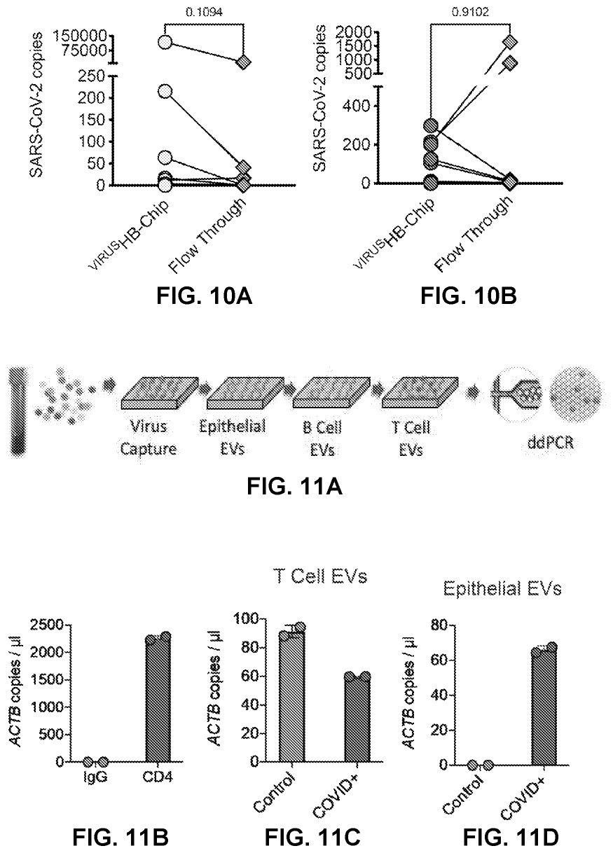

Further data are provided for virus isolation from saliva or stool samples (FIG. 10A-10B).

### Example 8: Analysis of Immune Vesicles from Plasma of Patients

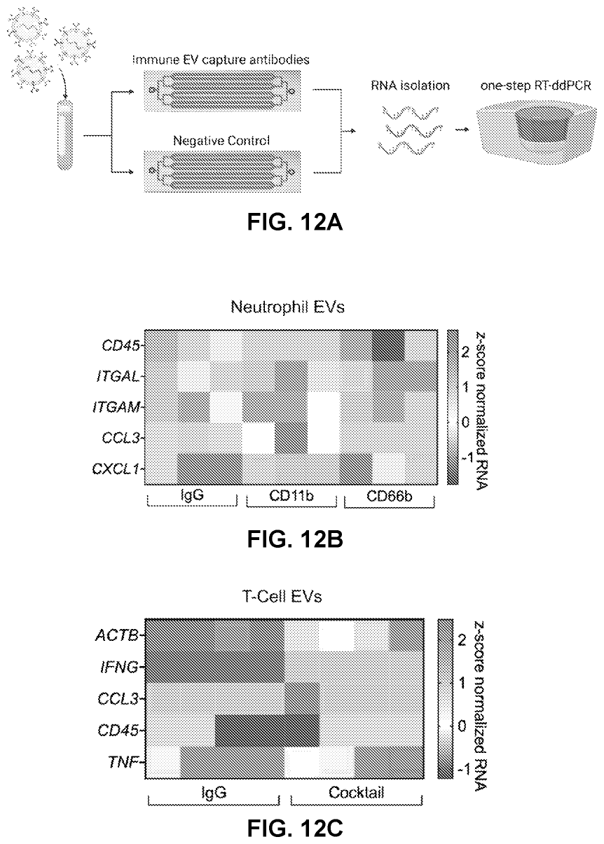

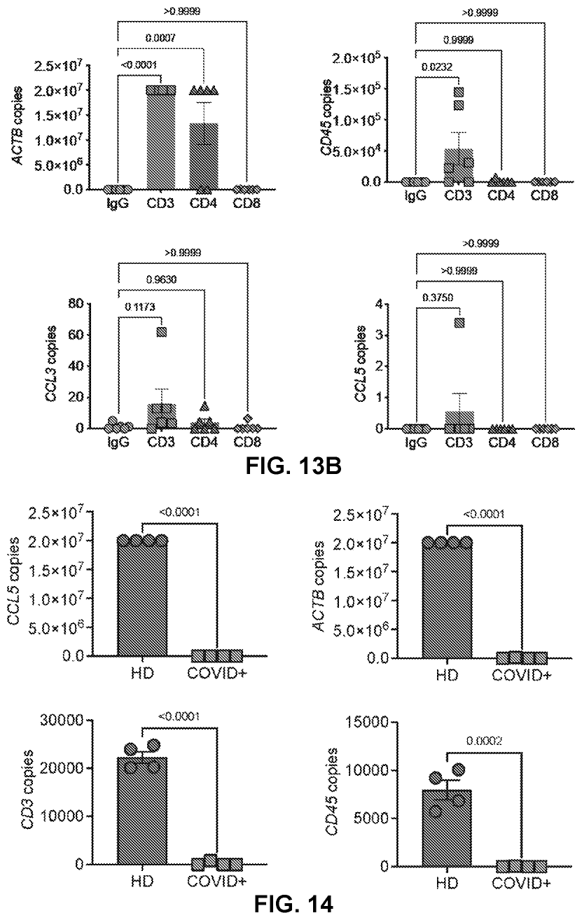

Through modification of the capture agents on the device surface, EVs can be selectively captured from unique cell populations. Further, when using an HB-Chip, the outlet (or exit tubing) of the device can be connected to an inlet (or input) of another chip with minimal sample loss (see, e.g., FIG. 11A). By attaching different capture agent to each chip, a single patient sample can be processed through multiple devices, with specific EVs captured on each chip (see, e.g., FIG. 11A or FIG. 13A). Towards identifying an EV-based signature for COVID patients, one or more of the following immune cell/antibody targets can be employed: T cells (e.g., SUPT1 cells with CD3/CD4 capture), B cells (e.g., JY cells with CD19/CD20 capture), and epithelial cells (e.g., SkBr3 and B5/589 with EpCAM/EGFR capture). EVs can be isolated from each cell line to test the specificity and sensitivity of the antibodies selected. Protein expression on the parental cells and EVs can be confirmed (e.g., by flow cytometry) prior to conducting capture experiments. EVs can be spiked into control plasma at varying concentrations (e.g., 1×10⁴, 1×10⁶, 1×10⁸ EVs/mL), and samples can be processed on individual chips, as well as in a system in which chips are placed in series.

Performance can be characterized using droplet digital PCR (e.g., by confirming enrichment of cell specific miRNAs and mRNAs), mass balance of EV counts (e.g., using qNANO or Exoid from Izon Science Ltd., Christchurch, New Zealand), and/or imaging of EVs on chip. Processing conditions (e.g., flow rate, dilution factor), capture agent selection, and capture agent concentration can be optimized (e.g., to achieve 80% capture efficiency) to provide desired enrichment (e.g., a minimum of 10× enrichment). Plasma from healthy controls can be processed to confirm the specificity and sensitivity of the assay prior to evaluating samples from the SARS-COV-2 cohort. For all conditions, EVs can be captured on the individual device surface (e.g., with a device ‘dead volume’ of 100 μL). Processing operations can be performed, such as one or more wash steps (e.g., to remove excess protein and surface bound nucleotides), on-chip RNA and protein extraction, cDNA creation, and amplification. ddPCR can be performed to analyze whether SARS-COV-2 viral RNA can be detected in infected patient extracellular vesicles, as well as cell markers for each cell type to determine if numbers of vesicles are altered between groups.

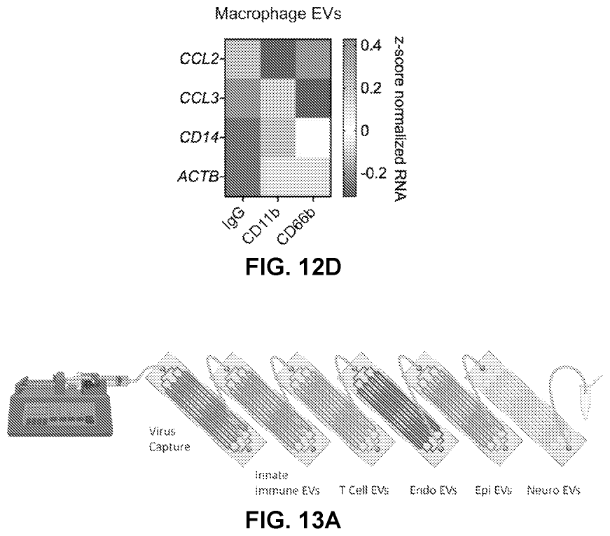

Cell line EVs were spiked into healthy plasma and captured on-chip (FIG. 12A). Cell-specific EVs were captured using antibodies as capture agents. RNA was isolated with the MagMAX mirVana total RNA kit and quantified using one-step RT-ddPCR (FIG. 12B-12D). A capture cocktail of CD3, CD4, and CD8 was used to target the T-cell population of interest (FIG. 13A-13B).

Preliminary ddPCR of EV RNA has shown higher levels of CD14, CD45, CXCL1, and IL1B in innate immune EVs of severe COVID patients compared to less severe patients. Additionally, T-cell EVs show higher levels of CCL5, CD3, and CD45 in patients with severe COVID. Using COVID-19 infection related EVs in addition to intact SARS-CoV-2 viral detection, on-chip assays have the potential to provide further insight into the potential infectivity and outcome for COVID-19 patients. These results indicate that EV analysis of COVID-19 patients has the potential to help predict disease severity and determine which patients are more likely to need intensive care and intervention.

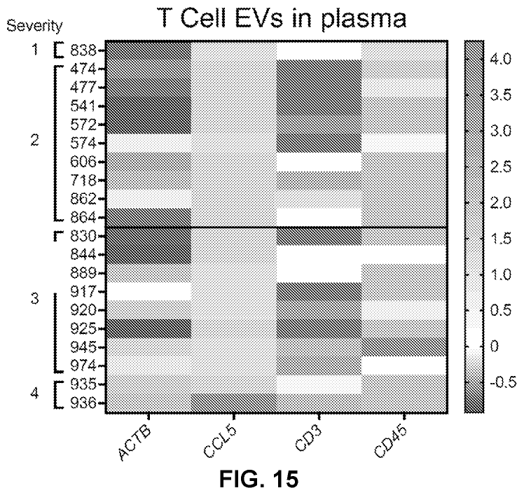

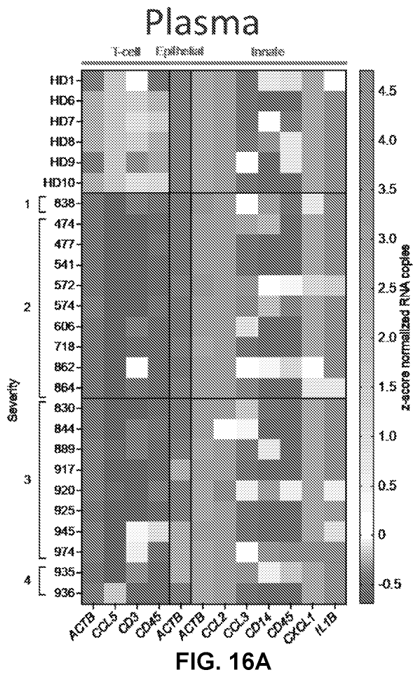

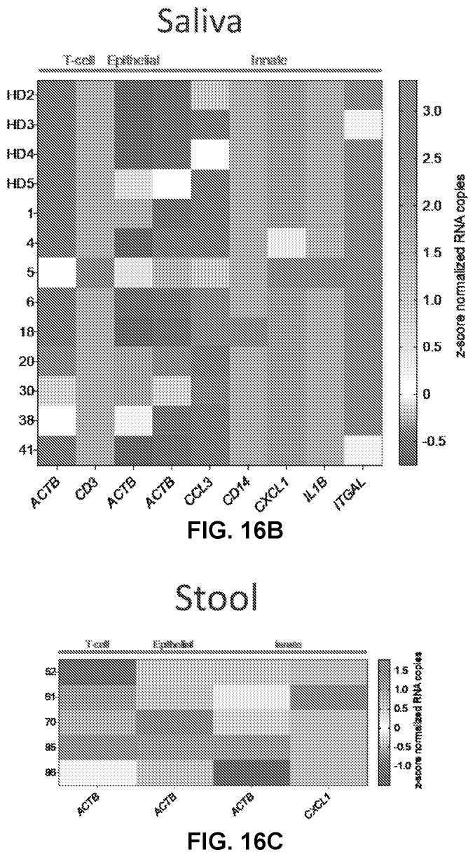

Studies have shown that T-cell EVs decrease while epithelial cell EVs increase between COVID− and COVID+ patients. By studying if these changes are observed in patient sample, validation studies can be performed to determine if EVs can serve as either a factor in SARS-COV-2 diagnosis when viral particles are present in very low number or if EVs are predictive of outcome in patients with SARS-COV-2 infection (see, e.g., FIG. 11B-11D). In some non-limiting instances, exosomal viral signatures may be employed to discriminate between diagnoses, as well as identify patients that may develop acute symptoms (e.g., such as Acute Respiratory Distress Syndrome (ARDS)) or that may require certain treatment (e.g., support by a ventilator). T-cell EVs were observed to be decreased in COVID+ patients (FIG. 14). Furthermore, severe COVID+ patients had a different EV signature (FIG. 15 and FIG. 16A-16C).

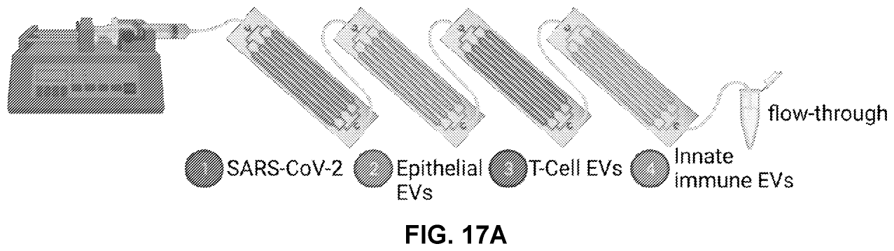

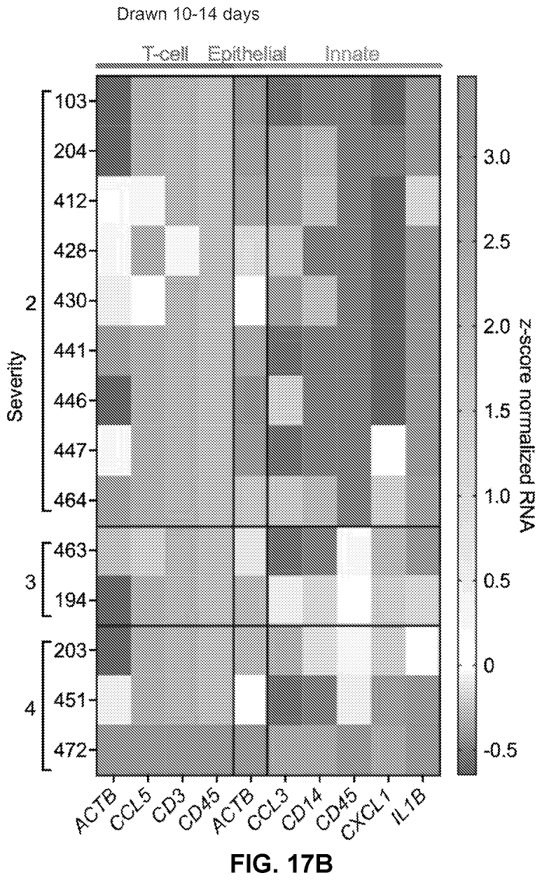

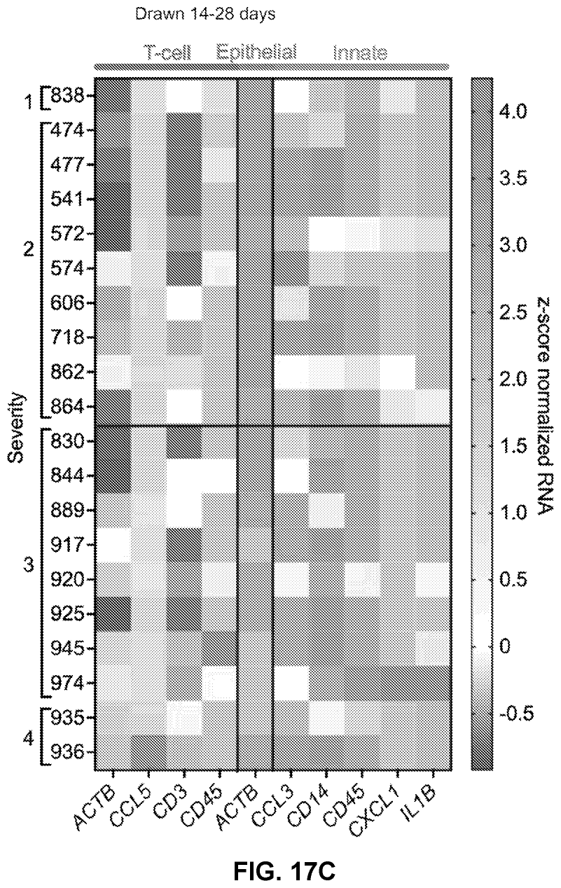

EVs were isolated using serial chip capture of T cell, epithelial, and innate immune cells using serially connected HB-Chips (FIG. 17A). Data are provided for samples drawn 10-14 days post-diagnosis (n=14) (FIG. 17B) or 14-28 days post-diagnosis (n=20) (FIG. 17C). RNA was reverse transcribed, pre-amplified using the SMART-Seq HT kit, and quantified through ddPCR. Severe outcome 3 (ICU admission) or 4 (death) were associated with increased T cell, epithelial, and innate immune EV signature. These results indicate that testing of immune cell EVs could be used to identify patient populations at risk of certain outcomes or certain disease progression.

### Example 9: Viral Load Compared to Clinical Metrics

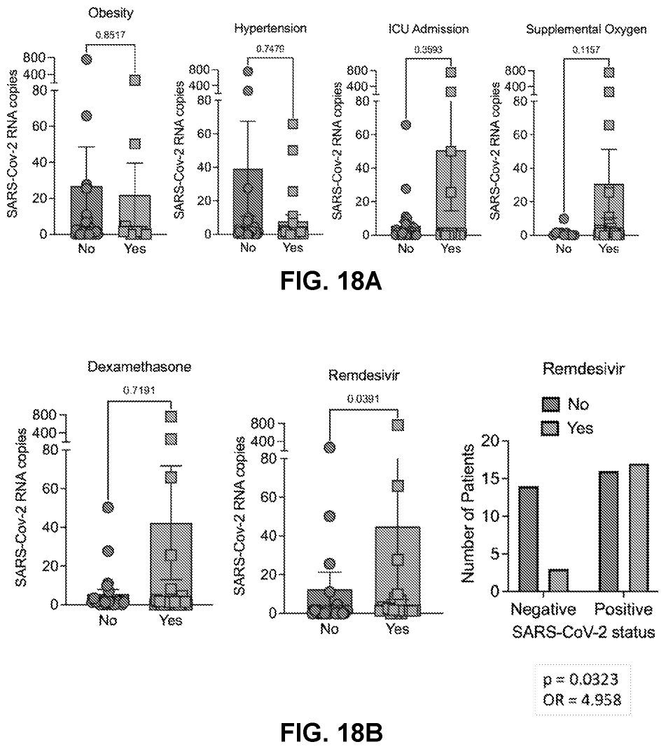

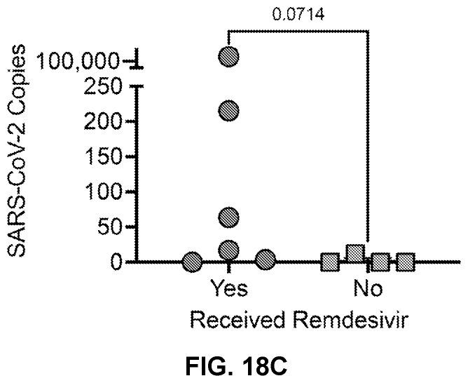

In a set of 50 plasma samples collected within two days of COVID-19 diagnosis, high viral load was predictive of remdesivir treatment, separate from other COVID-19 comorbidities. Viral load was compared to clinical characteristic and outcome (FIG. 18A-18C). Comorbidities associated with poor outcome (e.g., obesity or hypertension) were not correlated to high viral load in the tested patient cohort; and ICU admission and supplemental oxygen use have higher viral load, but not significant in the tested patient cohort (FIG. 18A). When comparing viral load to determine whether certain therapy is used, dexamethasone was not predicted in the patient set, and future remdesivir treatment seen in patients with a higher viral load with an odds ratio of 4.958 (FIG. 18B). Higher saliva viral load was observed in patients that later received remdesivir (FIG. 18C).

### Example 10: Use of Cell-Specific EV Capture for Immunotherapy

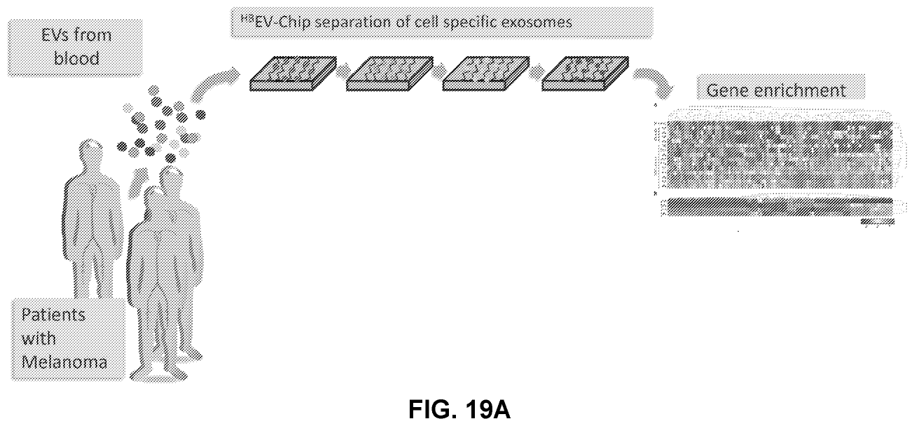

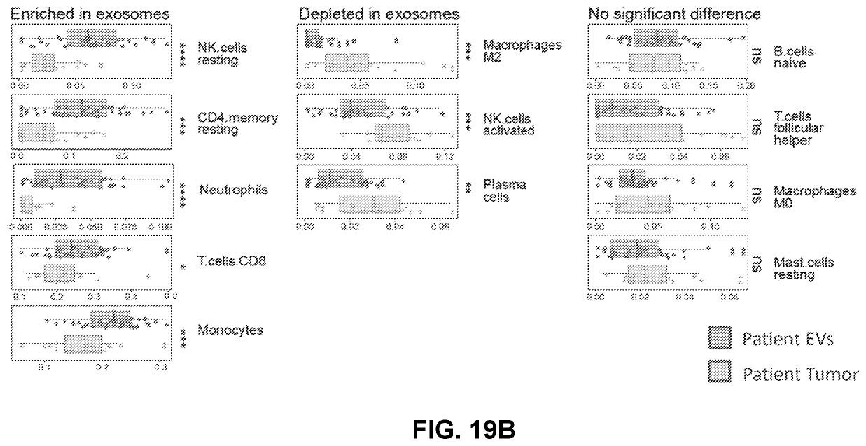

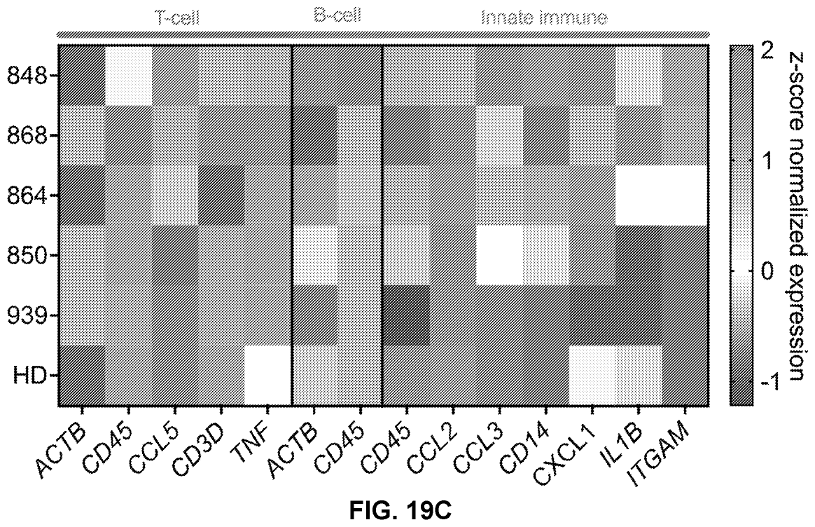

The microfluidic assays herein can be adapted for capturing any desired target cell or target EV. For instance, cell-specific EVs for immune cells can be captured by using capture agents that bind to certain cells (FIG. 19A). For instance, analysis of immune EV cells in melanoma patients indicate that certain exosomes are enriched in blood isolates, as compared to tumor tissue (FIG. 19B). Exosome signatures can be used as predictive markers that allow for non-invasive monitoring of tumor status and host immune status during immunotherapy. This could include capture of innate immune EVs using capture of CD11b and CD66b, T-Cell EVs using capture of CD3, CD4, and CD8, as well as B-Cell EVs using CD19, CD20, and HLA-DR capture. FIG. 19C shows non-limiting results from melanoma patients and one healthy donor, in which targets were captured using T-Cell, B-Cell, and Innate Immune EV chips.

### Example 11: Aryl-Diazonium Salts Offer a Rapid and Cost-Efficient Method to Functionalize Plastic Microfluidic Devices for Immunoaffinity Capture

Microfluidic devices have been used to isolate cells, viruses, and proteins using on-chip immunoaffinity capture strategies. To accomplish this, the inner surface of the chip can be modified to present binding moieties for the desired analyte. While this approach has been successful in research settings, it can be challenging to scale many surface modification strategies. Traditional polydimethylsiloxane (PDMS) devices can be effectively functionalized using silane-based methods, allowing for capture using biotinylated antibodies, proteins, or aptamers. However, it can require high labor hours, cleanroom equipment, and/or hazardous chemicals. Manufacture of microfluidic devices using plastics, including cyclic olefin copolymer (COC), allows chips to be mass produced, but most surface functionalization methods used with PDMS are not compatible with plastic. Described herein are methods to deposit biotin onto the surface of a plastic (COC) microfluidic chips using aryl-diazonium radicals. This method chemically bonds biotin to the surface, allowing for the addition of streptavidin nanoparticles to the surface. Nanoparticles increase the surface area of the chip and allow for proper capture moiety orientation. This process is faster rate than other methods, can be performed outside of a fume hood, and/or is very cost-effective using equipment readily available to laboratories. Additionally, this method allows for more rapid and scalable production of devices, including for diagnostic testing.

Microfluidic strategies can be adapted and developed to quickly and efficiently isolate or enrich numerous types of biomarkers, including cells, extracellular vesicles, and viruses using affinity-based capture approaches. Many lab-made microfluidic devices include a pliable elastomer, polydimethylsiloxane (PDMS), which can be bonded to a glass slide. While they are relatively easy to create, they can be limiting when considering scaling of devices for clinical or commercial use. Generating PDMS-glass devices is very labor-intensive and often requires production in a cleanroom environment and the use of toxic chemicals that must be handled in a glove box inside a chemical hood.

Injection molding is a manufacturing approach that is frequently used to make microfluidic devices at a rate of tens of thousands per day. While ideal for high volume production, the plastics used are challenging to chemically modify such that they are stable over time. Further, difficulties can arise when treating such surfaces, such as for providing antibodies or proteins that are covalently bound to the inner surface of a plastic microfluidic chip. Gels and polymers can be used in microfluidic devices to increase the ability to add functional groups to the inner surface. For instance, thermoresponsive and layer-by-layer deposition approaches are potential solutions for PDMS-based devices, but have yet to be applied to plastic-based chips. However, the inherent thickness of gel and polymer coating cannot conform to devices that have precise three-dimensional features, masking device features and reducing performance.

To chemically bond molecules onto the inner surface of devices, several strategies can be employed. Physical adsorption (or physisorption) of proteins onto the surface of PDMS devices can employ the hydrophobic nature of PDMS. To account for the non-specific nature of this process, often reagents are used at significantly higher concentrations (often 10×) to account for the less efficient deposition process. This can result in prohibitively higher costs, higher variation in coating density, and/or less stable surfaces. Consequently, to create stable, covalent bonding to the inside surface of the device, a free carboxyl group on the surface of the device can be created and employed. This can be achieved either through oxygen plasma, ultraviolet/ozone, or piranha solution (e.g., H₂SO₄ and H₂O₂ at a ratio of 3:1 to 7:1) treatment of the surface. The free carboxyl group can then be reacted with silane to allow further functionalization of proteins to the surface of the device. However, silane is a highly toxic compound, which must be handled in a nitrogen filled glove box with a fume hood to avoid reaction with water prior to use. Depending on the form of silane used, the surface can then be functionalized with NeutrAvidin, proteins, particles (e.g., nanoparticles), or combinations of these. Other methods can employ ultraviolet/ozone treatment of plastics to create a free carboxyl group, which can be reacted to the primary amine of a linker. However, not all these methods can be translated to injection molded plastics, can be time consuming, and/or require use of specialized equipment in a class 1000 cleanroom.

Aryl-diazonium salts can be employed for functionalizing a carbon surface with aryl radicals through the reduction of a diazonium salt. In some non-limiting instances, aryl diazonium salts can be created chemically by reacting a phenolic compound (e.g., NH₂—C₆H₄—R) with sodium nitrite or nitrous acid to form an aryl diazonium salt (e.g., ⁺N₂—C₆H₄—R). Then, with an electron donor source supplied through either an electric current, UV-light, or ultrasonic stimulation, N₂ gas is released, and a C₆H₄-containing radical (e.g., a C₆H₄—R radical) can be formed. This radical can then readily react with inert surfaces including gold, carbon, or plastic forming a covalent bond with the surface of the device.

As described herein, aryl-diazonium salts can function as a relatively inexpensive, stable, and/or consistent source of surface functionalization of cyclic olefin copolymer (COC) plastic microfluidic devices using a UV-light bed. In particular, the reaction strategy can include an efficient reaction of biotin-NHS-esters with aryl-diazonium. A p-phenylenediamine can be reacted with sodium nitrite, which provides a free NH₂ group to react with a biotin-NHS-ester (FIG. 20A). This strategy effectively and evenly coats the entire surface of a microfluidic device within one hour. The process is highly reproducible and does not require access to a cleanroom. The absence of highly volatile chemicals, such as silane, allows for the protocol to be completed on a laboratory bench, if desired. When compared to other methods of functionalizing plastic devices, a dramatic increase of the surface binding capacity of the device was observed, leading to higher levels of analyte capture. In addition to being an effective strategy for chemically modifying plastic microfluidic devices, this strategy is also compatible with PDMS-glass devices. Overall, described herein is an easy, effective, and inexpensive method for functionalizing plastic COC microfluidic devices, leading to higher capture rates and longer stability at room temperature, thereby allowing for easier distribution and scaling.

### Example 12: Non-Limiting Experimental Details

Microfluidic device: A multichannel, single inlet and outlet, microfluidic device was employed, which is referred herein as a non-limiting herringbone chip (HB-Chip). This device has a higher aspect ratio of its inner features (e.g., >1) and a complex three-dimensional geometry, which can provide beneficial fluidic flow and quickly highlights the limitations of any surface modification strategy. Further, this device can be produced with identical features using PDMS-glass methods and injection molding. Injection molded HB-Chips were commercially produced by thinXXS Microtechnology (Germany).

Plastic aryl-diazonium devices: Plastic herringbone chips (FIG. 20B) were inspected for debris and imperfections. 20 mM p-phenylenediamine (Sigma, P6001) in 1 M hydrochloric acid (HCl, Sigma, 258148) and 20 mM sodium nitrite (Sigma, 237213) solution was reacted with EZ-link biotin-NHS-ester (final concentration of 10 mM, Pierce, 20217) for 30 minutes at room temperature to form a biotin aryl-diazonium salt (FIG. 20A). Devices were then flushed with two device volumes of the biotin aryl-diazonium solution through the inlet and exposed to UV light using a UV light bed (UVP 95042001) set to high for 10 minutes. UV light allows for creation of biotin aryl radical intermediates that then react with the plastic surface of the device. Devices were then flushed with five device volumes of ethanol (EtOH, Sigma, 493546) to removed bubbles, followed by five device volumes of PBS (Corning, MT21040CV) through the inlet. Another two device volumes of the biotin aryl diazonium intermediate solution were flushed through the outlet of each device followed by another 10-minute UV exposure. Devices were flushed with five device volumes of ethanol to remove bubbles, followed by 10 device volumes of air to dry them. Herringbone chips were then stored at 25° C. in a vacuum desiccator until used. Prior to use, devices were flushed with two device volumes of a 0.01667% solution of streptavidin nanoparticles in PBS through the inlet of the devices. After a 15-minute incubation, two device volumes of streptavidin nanoparticles were flown through the outlet of the device. Chips were then used immediately or capped and stored up to one week at 4° C.

Plastic physisorption devices: Plastic herringbone chips (FIG. 20C) were inspected for debris and imperfections. Devices were then flushed with five device volumes of EtOH through the inlet, followed by ten device volumes of PBS through the inlet. Four device volumes of 1 mg/ml NeutrAvidin diluted in PBS were flushed through the inlet of the devices, followed by a 30-minute incubation at room temperature. The process was repeated by flushing another four device volumes of NeutrAvidin through the outlet of each device and incubating at room temperature for 30 minutes.

PDMS-glass device—Silane functionalization: Glass-polydimethylsiloxane (PDMS) microfluidic chips were produced. Briefly, in a class 1000 cleanroom, the PDMS and glass surfaces were exposed to oxygen plasma for seven minutes (March Instruments, PX-250) then placed together and put on a hot plate for ten minutes. Within 30 minutes of bonding, the devices were brought into a chemical hood where a 4% (w/v) solution of 3-mercaptopropyl trimethoxysilane (Silane, Gelest, SIM6476.0) in EtOH (FIG. 20C) was manually pushed through the chip using a syringe. Four device volumes of 100 μg/ml N-γ-maleimidobutyryl-oxysuccinimide ester (GMBS, Pierce, 22309) diluted in EtOH were flushed through the inlet and outlet of each device. After a 15-minute incubation period, the GMBS addition was repeated. Following, devices were washed with five device volumes of EtOH, alternating between the device inlet and outlet. Devices were then flushed with five device volumes of 20 μg/mL NeutrAvidin (Thermo Scientific, 31050) diluted in phosphate buffered saline (PBS), again alternating between the inlet and outlet. After a 30-minute incubation at room temperature, the NeutrAvidin addition was repeated.

PDMS-glass device—Aryl-diazonium functionalization: Following device bonding (see above), 20 mM p-phenylenediamine in 1 M HCl and 20 mM sodium nitrite solution were reacted with biotin-NHS-ester (final concentration of 10 mM) for 30 minutes at room temperature to form a biotin aryl diazonium salt (FIG. 20D). Devices were then flushed with two device volumes of the biotin aryl diazonium solution through the inlet and exposed to UV light using a UV light bed (UVP 95042001) set to high for 10 minutes. UV light allows for creation of biotin aryl radical intermediates that then react with the plastic surface of the device. Devices were then flushed with five device volumes of ethanol to removed bubbles, followed by five device volumes of PBS through the inlet. Another two device volumes of the biotin aryl diazonium intermediate solution were flushed through the outlet of each device followed by another 10-minute UV exposure. Devices were flushed with five device volumes of ethanol to remove bubbles. Devices were flushed with five device volumes of PBS. Then two device volumes of a 0.01667% solution of streptavidin nanoparticles were flown through the inlet of the devices. After a 15-minute incubation, two device volumes of streptavidin nanoparticles were flown through the outlet of the device.

R-phycoerythrin (RPE) assay: Devices were flushed with five device volumes of PBS per side and blocked with five device volumes of Intercept (TBS) Blocking Buffer (LICOR, 927-60001). For each device, 10 μL of R-Phycoerythrin (R-PE), Biotin-XX Conjugate (ThermoFisher, P811) in 990 μL 1% BSA (Sigma, A3059) in PBS was flown through each device at 2 mL/hour using a PhD ULTA syringe pump (Harvard Apparatus) protected from light. Devices were incubated at room temperature, protected from light for 30 minutes. Devices were then flushed with 2.5 mL PBS at 2.5 mL/hour using a syringe pump. Nine representative images were taken per device using a Nikon Eclipse 90i microscope with a 10× lens and Andor camera [Model #DR-328G0C01-SIL] with a neutral density 4 (ND4) filter. A TexasRed filter was used, and 100 millisecond (ms) exposure images were taken. Using NIS-Elements, the average fluorescent intensity of each image was measured. Background fluorescence (with no device present) was recorded and subtracted from all values.

### Extracellular Vesicle (EV) Capture on Devices

Cell Culture: MDA-MB-231-BM1 (BM1) cells and MDA-MB-468 cells were employed. BM1 and HEK-293T cells (ATCC, CRL-3216) were propagated in Dulbecco's Modified Eagle Media with glutamine and 4.5 g/L glucose (Corning, 10-013-CV) supplemented with fetal bovine serum (FBS), qualified (Gibco, 26140-079) at a final concentration of 10% and penicillin, streptomycin (P/S, Gibco, 15140163) at a final concentration of 1% at 37° C. with 5% CO₂. MDA-MB-468 Cells were propagated in RMPI-1640 media with glutamine (Corning, 10-040-CV) supplemented with FBS at a final concentration of 10% and P/S at a final concentration of 1% at 37° C. with 5% CO₂. Cells authentication was performed by short tandem repeat analysis compared to the primary MDA-MB-231, HEK-293, and MDA-MB-468 genotypes respectively and cells were checked for Mycoplasma prior to use and every 6 weeks following using the MycoAlert test (Lonza, LT07-318).

Lentiviral Transductions: To fluorescently label EVs, MDA-MB-231 BM1 and MDA-MB-468 cells were transduced with a palmitoylated-tdTomato fluorescent reporter using lentivirus. Third generation lentiviruses were propagated under BL2+ conditions as approved by the Mass General Brigham Institutional Biosafety Committee. pMDLg/pRRE, pRSV-Rev, pVSV-G lentiviral packaging plasmids were combined with pCSCGW2-PalmtdTomato lentiviral vector and TransIT-Lenti (Mirrus, MIR6600) to transfect HEK-293T cells per the manufacturer's protocol. Lentiviral media was collected after 48 hours and filtered through a 0.45 μm filter. 1 μL of TransduceIT Transduction Reagent (Mirrus, MIR6620) was added per 1 mL of lentiviral media. 1.5 mL of viral containing media was then added to transduce cells over 24 hours. Transduced cells were then selected for viral expression of palmitoylated-tdTomato by flow cytometry following transduction.

Antibody biotinylation for EV Capture: Antibodies were incubated at room temperature while rotating with Biotin PEG SCM 2 kDa (Creative PEGworks, PJK-1900) for two hours at a molar ratio of biotin linker: antibody of 20:1. Excess biotin linker was removed using Zeba Desalting Columns (Thermo Scientific, 89882). Antibodies were then aliquoted for single use and stored at −80° C.

Extracellular Vesicle (EV) Capture: MDA-MB-231-BM1, and MDA-MB-468 cells were grown to 90% confluence in 15-cm dishes. They were then washed three times with PBS, to remove any media containing FBS. Cells were incubated in serum free media containing 1% P/S for 48 hours in a 37° C. incubator to collect secreted EVs. Conditioned media was removed from the cells and spun at 2,000×g for 10 minutes to remove any cells, debris, or apoptotic bodies. Media was then concentrated 10-fold using 10-kDa Amicon Ultra-15 filters (Millipore, UFC901024).

For all devices, 2 device volumes of a 20 μg ml⁻¹ solution of either an anti-EGFR antibody (Eli Lilly, Cetuximab) or non-specific IgG (BioLegend, 401402) were added to inlet of each device, and incubated at room temperature for 30 minutes. Then two device volumes of the same antibody were then flown through the outlet of the same device. After a 30 min incubation, devices are blocked with two device volumes of Intercept (TBS) Blocking Buffer (LICOR, 927-60001).

For EV capture alone, 500 μL of 10× concentrated conditioned media was flown through each device. For experiments with normal plasma, 50 μL of 30× concentrated EVs were added per 500 μL of normal human plasma. 500 μL of EV-spiked plasma was flown through a herringbone capture device at 1 mL hour⁻¹. Devices were then washed with 1.5 mL of PBS flown through at 1.5 mL hour⁻¹.

EV Imaging: After washing with PBS, devices were capped and then imaged on a Nikon Eclipse 90i microscope with a 10× lens and Andor camera [Model #DR-328G0C01-SIL], with ND4 and TexasRed filters. Nine representative images were taken per device with a one-second(s) exposure time. Using NIS-Elements, the total fluorescent intensity of each image was measured. Fluorescent intensity values were then normalized to the IgG, no EV devices to show ΔtdTomato signal from background chip fluorescence.

RNA Extraction: RNA was extracted from devices using the MagMAX mirVana Total RNA Isolation Kit (Applied Biosystems, A27828). For each device 99 μL Lysis Buffer (from A27828)+100 μL Isopropanol (Fisher Chemical, A451SK-1)+1 μL β-mercaptoethanol (Sigma Aldrich, M3148) was flown through 12 times by manually pushing between syringes attached to the inlet and exit port of devices. RNA was then isolated with DNase treatment per the manufacturer's manual extraction protocol (Applied Biosystems, A27828).

One-Step Reverse Transcription and ddPCR: RNA levels were measured using the 1-Step RT-ddPCR Advanced Kit for Probes (Bio-Rad, 1864021) and pre-designed primer/probe mixes for each gene (Integrated DNA Technologies, IDT). Reactions were performed using 5.5 μL RNA per reaction with 500 nM of primers (final concentration) and a primer: probe ratio of 4:1. Droplet generation was performed on the QX200 AutoDG, PCR amplification on the C1000 Touch Thermal Cycler, droplet reading on the QX200 Droplet Reader, and analysis using QX Manager (Bio-Rad).

Primer and Probe Sequences (Integrated DNA
Technologies, IDT)
ACTB Assay: Hs.PT.39a.22214847
ATCB-F:
(SEQ ID NO: 1)
CCTTGCACATGCCGGAG
ACTB-R:
(SEQ ID NO: 2)
ACAGAGCCTCGCCTTTG
ACTB-Probe:
(SEQ ID NO: 3)
TCATCCATGGTGAGCTGGCGG
CCL5 Assay: Hs.PT.58.40305992
CCL5-F:
(SEQ ID NO: 4)
GACTCTCCATCCTAGCTCATCT
CCL5-R:
(SEQ ID NO: 5)
GAGTATTTCTACACCAGTGGCA
CCL5-Probe:
(SEQ ID NO: 6)
ATGTACTCCCGAACCCATTTCTTCTCTG
CD14 Assay: Hs.PT.56a.3118607.g
CD14-F:
(SEQ ID NO: 7)
AATCTTCATCGTCCAGCTCAC
CD14-R:
(SEQ ID NO: 8)
CAGAGGTTCGGAAGACTTATCG
CD14-Probe:
(SEQ ID NO: 9)
CGCAGAGACGTGCACCAGC
CNTRL Assay: Hs.PT.58.1241761
CNTRL-F:
(SEQ ID NO: 10)
CATTTTCCACCTCCGTTCATTG
CNTRL-R:
(SEQ ID NO: 11)
GTCTCTTTCCAGTCTTTCTACCTC
CNTRL-Probe:
(SEQ ID NO: 12)
TTGGAAGGTCAGCCAGTAACCACTC
IL1A Assay: Hs.PT.58.2851435
IL1A-F:
(SEQ ID NO: 13)
TCTTCATCTTGGGCAGTCAC
IL1A-R:
(SEQ ID NO: 14)
GCTGCTGCATTACATAATCTGG
IL1A-Probe:
(SEQ ID NO: 15)
TGAAGCAGTGAAATTTGACATGGGTGC
IL20RB Assay: Hs.PT.58.39994983
IL20RB-F:
(SEQ ID NO: 16)
GACCTTCAGTGAGTGAGCAC
IL20RB-R:
(SEQ ID NO: 17)
ACCAACATGAAGCATCTCTTGA
IL20RB-Probe:
(SEQ ID NO: 18)
AGCCTGTACACGAGCCACATCTG
GAPDH Assay: Hs.PT.39a.22214836
GAPDH-F:
(SEQ ID NO: 19)
TGTAGTTGAGGTCAATGAAGGG
GAPDH-R:
(SEQ ID NO: 20)
ACATCGCTCAGACACCATG
GAPDH-Probe:
(SEQ ID NO: 21)
AAGGTCGGAGTCAACGGATTTGGTC
SLPI Assay: Hs.PT.58.3977822
SLPI-F:
(SEQ ID NO: 22)
TGTGGAAGGCTCTGGAAAG
SLPI-R:
(SEQ ID NO: 23)
TGGCACTCAGGTTTCTTGTATC
SLPI-Probe:
(SEQ ID NO: 24)
TGGGCAGATTTCTTAGGAGGACAGACT
FLYWCH1 Assay: Hs.PT.58.40054436.g
FLYWCH1-F:
(SEQ ID NO: 25)
CCAGCCAGCCCTAGAGAT
FLYWCH1-R:
(SEQ ID NO: 26)
CACTGCCTTCTCCTGCTTG
FLYWCH1-Probe:
(SEQ ID NO: 27)
AGGAAGGACTCCAGCACCAGGA

Patient plasma collection: Plasma was collected from healthy donors in BD Vacutainer PPT Plasma Preparation Tubes (BD Biosciences, 362788) according to a protocol approved by the Institutional Review Board (IRB).

### Example 13: Non-Limiting Aryl-Diazonium Reaction Strategy

The lack of covalent binding can result in a less stable surface coating. Described herein is a reaction strategy for depositing a biotinylated aryl-diazonium directly onto the surface of a device (e.g., a cyclic olefin copolymer (COC) plastic device). Briefly, p-phenylenediamine and sodium nitrite can be reacted with biotin-NHS-ester to create a biotin-aryl-diazonium salt. This solution is then flowed into the devices, where UV light is used to produce a biotin-aryl radical through introduction of electrons and loss of N₂ gas. This radical is then able to efficiently react with all plastic surfaces of the herringbone device, coating it in biotin (FIG. 20).

### Example 14: Optimization of Aryl-Diazonium Reaction

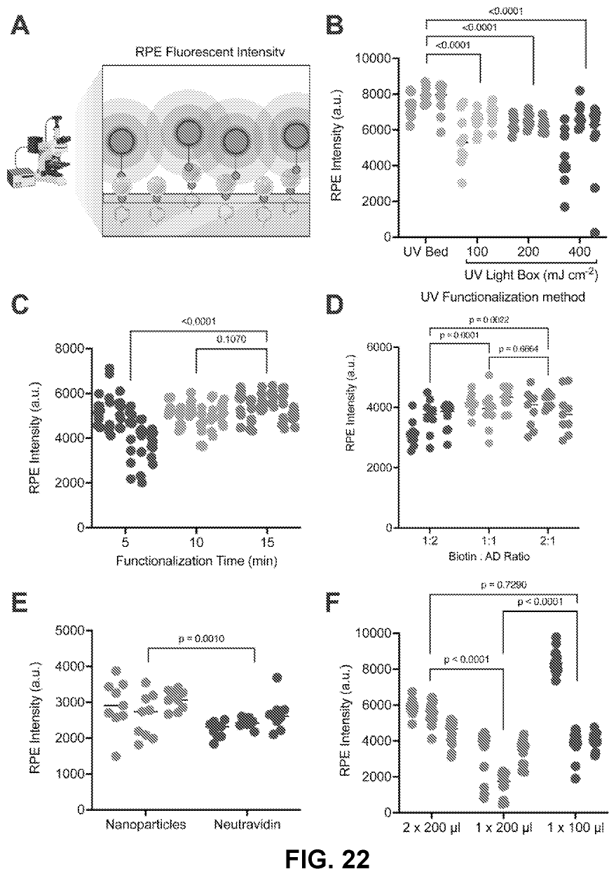

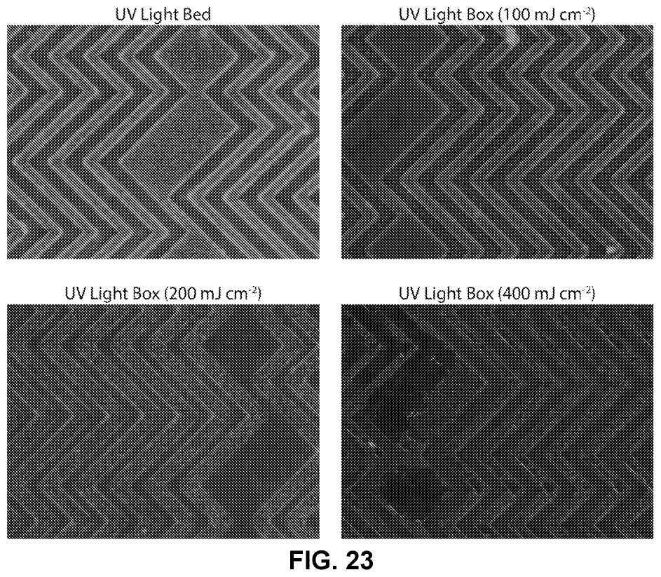

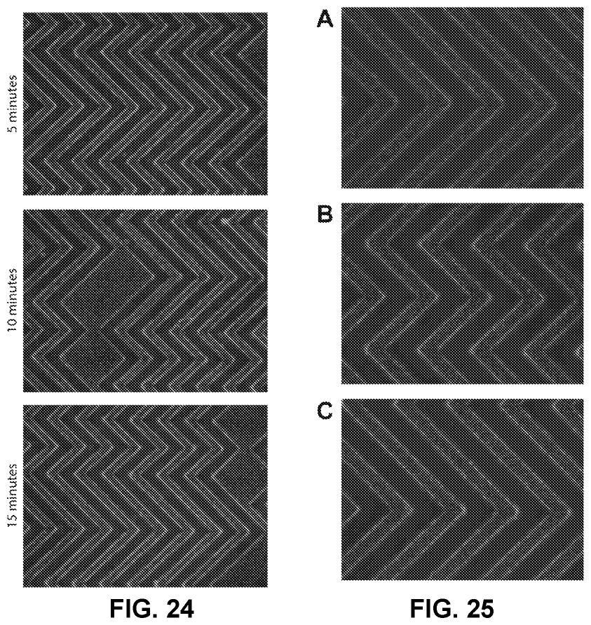

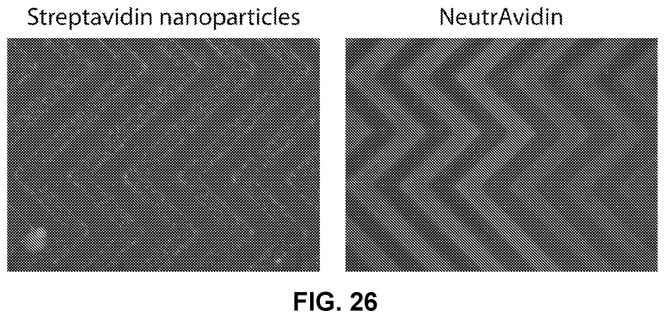

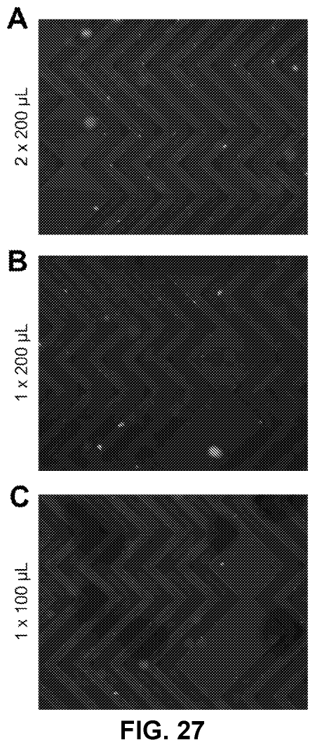

To optimize functionalization of biotin to surfaces within device, a visual assay was used to assess the binding capacity of the surface coatings (FIG. 20A). After biotin functionalization of the surface, streptavidin nanoparticles are added to the device. Following this, a fluorescently tagged biotin molecule (biotin R-phycoerythrin, R-PE) was flown through the devices, such that this red fluorescent reporter would bind to available streptavidin coated nanoparticles on the surface. Once tagged, nine random points were then imaged to determine average fluorescent intensity of the devices. During early optimization of the functionalization strategy, allowing the biotin-NHS-ester to react with the aryl diazonium in solution led to a dramatic increase in the coating of biotin across the surface of the device. Additionally, increasing the concentration of aryl diazonium above 20 mM led to a decrease in biotin functionalization to the surface (FIG. 21). To further test reaction efficiency, various parameters were tested, including the UV source, UV exposure time, ratio of biotin to aryl-diazonium, solution volume, as well as nanoparticles versus NeutrAvidin. When compared functionalization using a UV light box vs a UV light bed, the UV bed produced the most robust and consistent reaction efficiency (FIG. 22B and FIG. 23). Exposure of the devices to UV light for 10 minutes significantly increased binding capacity of the devices, with further exposure time showing no increase in binding capacity (FIG. 22C and FIG. 24). A ratio of biotin to aryl-diazonium of 1:1 showed the best reaction efficiency with increased biotin showing no increase in binding (FIG. 22D and FIG. 25). Using streptavidin coated nanoparticles can increase the binding capacity of the device to both cells as well as extracellular vesicles. When binding capacity with NeutrAvidin to streptavidin nanoparticles was compared, results with streptavidin coated nanoparticles produced increased levels of binding capacity (FIG. 22E and FIG. 26). Also tested was the amount of solution required to flow through the device for optimum binding. Flowing just one round of 200 μL showed the least surface functionalization. Flowing 100 μL through twice showed increased levels of binding capacity, but increased inconsistency between and within devices. Flowing 200 μL of biotin-aryl-diazonium through the device twice produced the highest and most consistent levels of binding (FIG. 22F and FIG. 27).

### Example 15: Device Stability

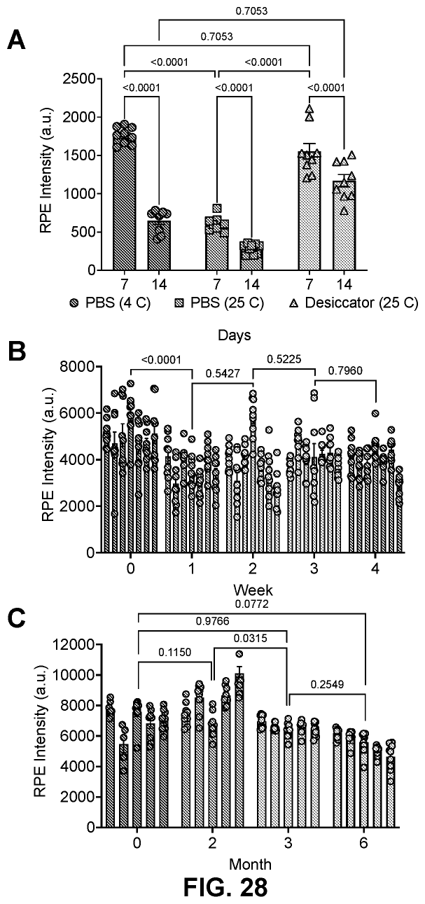

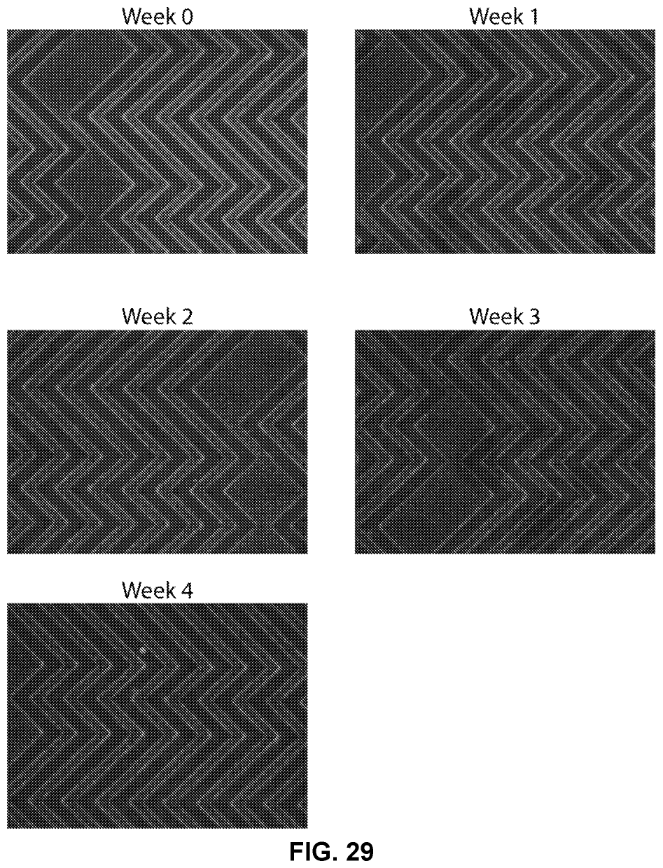

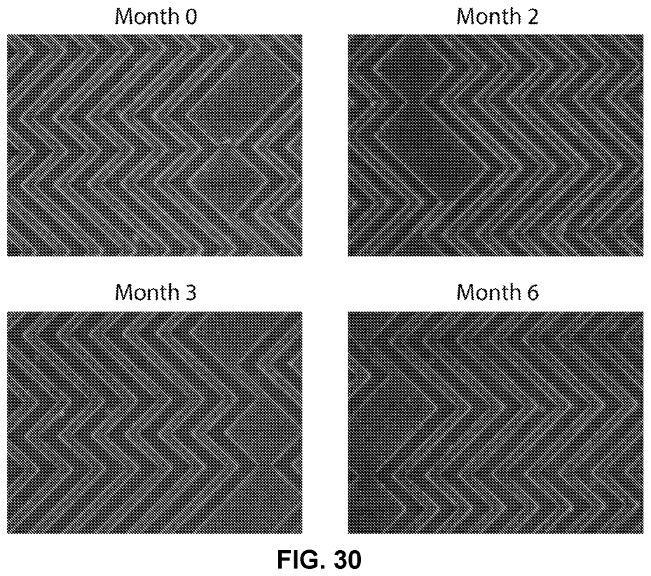

Previous methods of functionalizing plastic devices relied on cold storage in PBS at 4° C. The methods herein for chemical functionalization was tested to determine whether the same storage conditions were required. Following storage, streptavidin nanoparticles were added, and surface binding was assessed through the RPE assay. When storage in PBS at 4° C. to 25° C. was compared, a significant decrease in surface binding of the device stored at room temperature was observed. However, storage of dry devices in a desiccator at 25° C. showed no significant drop in surface binding with a slight increase in stability over time (FIG. 28A). Device stability was tested over time stored in a desiccator. When examined at day 8 after functionalization compared to day 1, a 29% decay in binding capacity was observed. However, subsequent weekly measurements saw no significant decay in binding capacity of devices stored dry in desiccant at 25° C. (FIG. 28B and FIG. 29). For longer term storage experiments, dried devices were stored in a vacuum desiccator for the number of months shown before nanoparticles were added and surface binding was assessed by an R-PE assay. When devices were stored in a vacuum desiccator up to six months, no loss in binding capacity of devices was observed (FIG. 28C and FIG. 30). This further supports the use of this method for functionalizing devices that can be mass produced and shipped to labs for use either as research products or diagnostic tools in clinical labs. Because of their stability at room temperature, production and shipping of these devices will be much easier, allowing for more rapid and cost-effective dissemination.

### Example 16: Aryl-Diazonium Devices have Increased Binding Compared to Other Methods

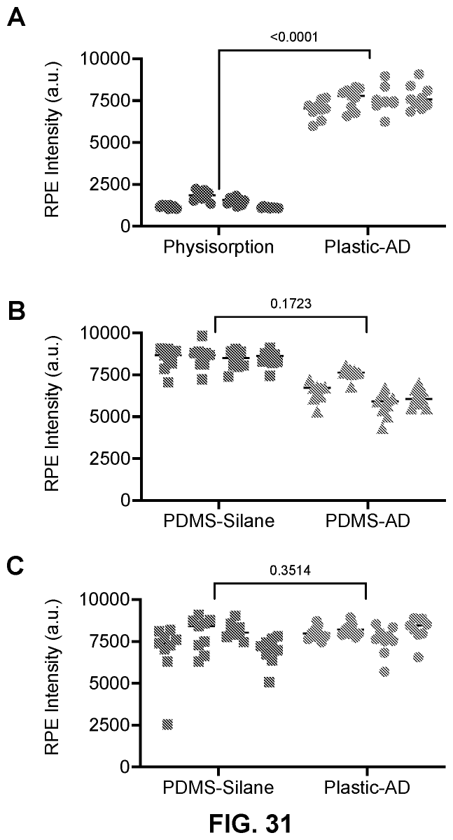

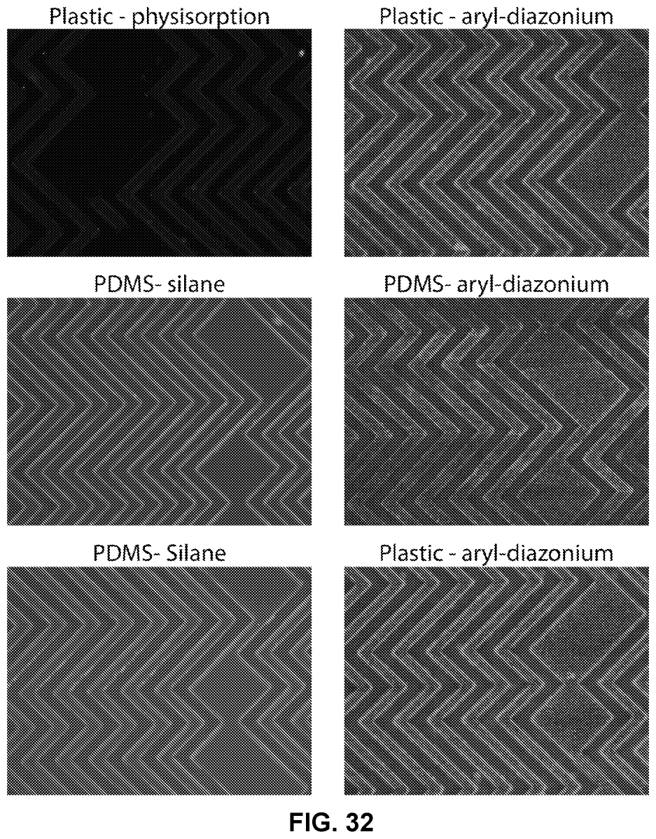

To determine performance, the present method was compared to other methods (FIG. 20B-20E). PDMS devices functionalized with silane can be used to coat the surface of the device with NeutrAvidin. These devices showed high rates of capture, uniform coating, and high stability stored dry in desiccant at 25° C. They additionally require the use of glove boxes and highly toxic chemicals for functionalization. To move to a scalable version of the herringbone device, COC plastic devices were produced using injection molding techniques. One strategy developed for the use of plastic devices involved direct deposition of NeutrAvidin to the surface (physisorption) (FIG. 20D). While these devices work for some applications, the method typically requires significantly higher concentrations of reagents (e.g., NeutrAvidin) to compensate for the less efficient process. This method showed uneven binding at significantly lower levels than the aryl-diazonium method (FIG. 31A and FIG. 32). When the binding capacity of PDMS devices functionalized with GMBS and NeutrAvidin was compared to both PDMS and plastic devices functionalized with Aryl Diazonium and streptavidin nanoparticles, no difference in binding capacity was observed using the RPE assay (FIG. 31B-31C and FIG. 32). Overall, the aryl-diazonium method shows more consistency of binding across the device surface, between devices, and between different batches of devices. Additionally, similar levels of binding to widely used PDMS silane and GMBS functionalization methods were observed, suggesting equivalency for use with immunoaffinity capture applications with patient biofluids. Because of the ability to mass produce plastic devices, the ease of functionalization, and the long-term stability at ambient temperatures, these devices are likely to serve as an improved method for analyzing small biomolecules and nanoparticles from patient samples.

### Example 17: Aryl-Diazonium HB-Chip Bind Tumor EV's at a Higher Rate

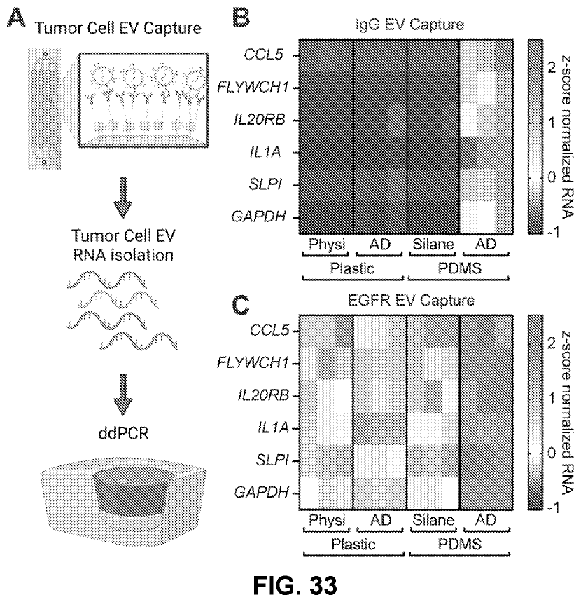

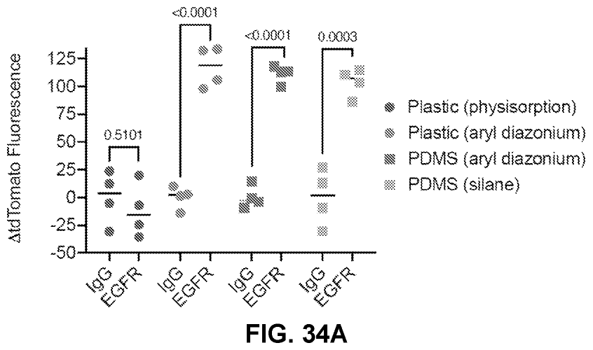

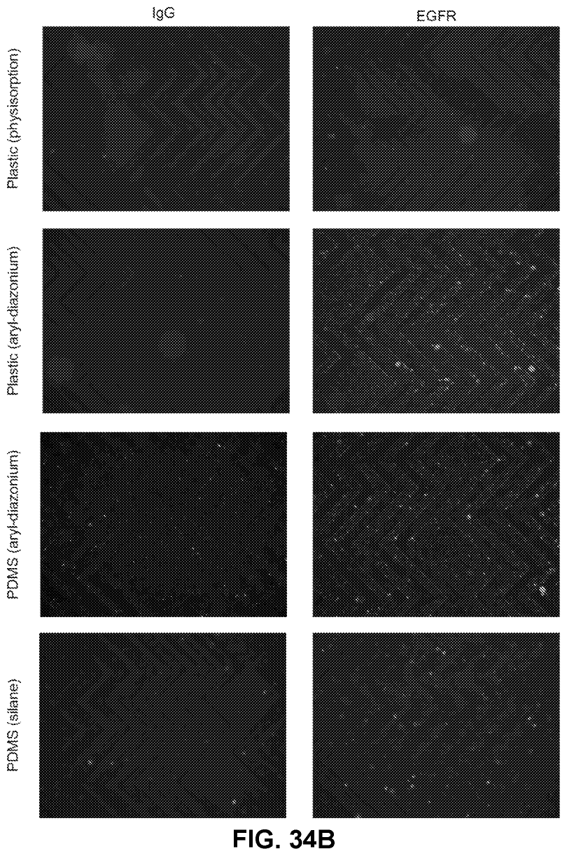

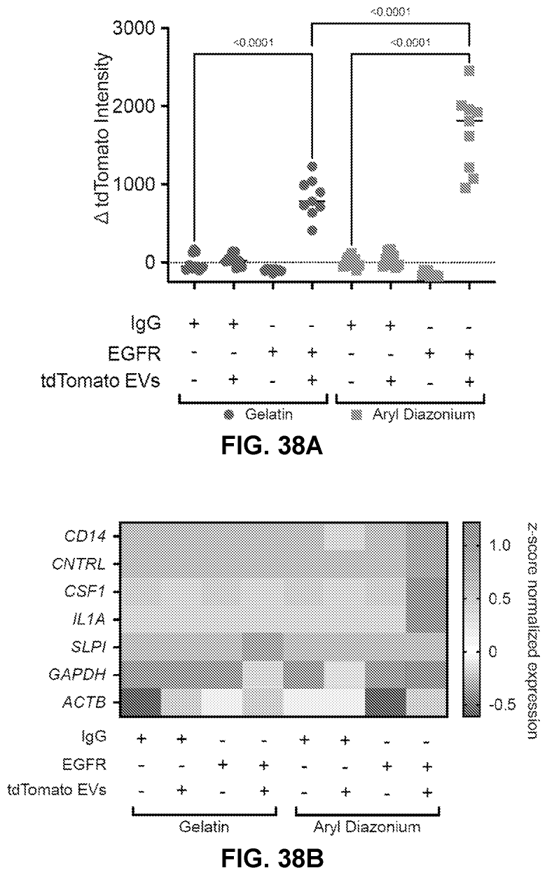

As the functionalization method showed higher rates of surface capture in the R-PE assay, the capability of aryl diazonium functionalized devices to bind tumor EVs was compared to silane-GMBS or physisorption functionalized devices. To do this, serum-free concentrated conditioned media from MDA-MB-231-BM1 tumor cells, containing palmitoylated tdTomato tagged EVs, were flowed through the functionalized devices using a syringe pump. RNA was extracted directly from the devices and analyzed for known EV RNA markers by ddPCR (FIG. 33A and FIG. 34). When non-specific binding of EVs was examined by using an IgG antibody, the aryl diazonium PDMS devices exhibited non-specific capture of EVs (FIG. 33B and FIG. 34). Using specific capture of EGFR+ EVs with the EGFR/EGFRvIII targeting antibody Cetuximab, aryl diazonium functionalized plastic and PDMS devices had higher rates of EV capture compared to GMBS-silane functionalized PDMS and physisorption functionalized plastic devices (FIG. 33C and FIG. 34). Also provided is a comparison with gelatin-coated devices (FIG. 38A-38B).

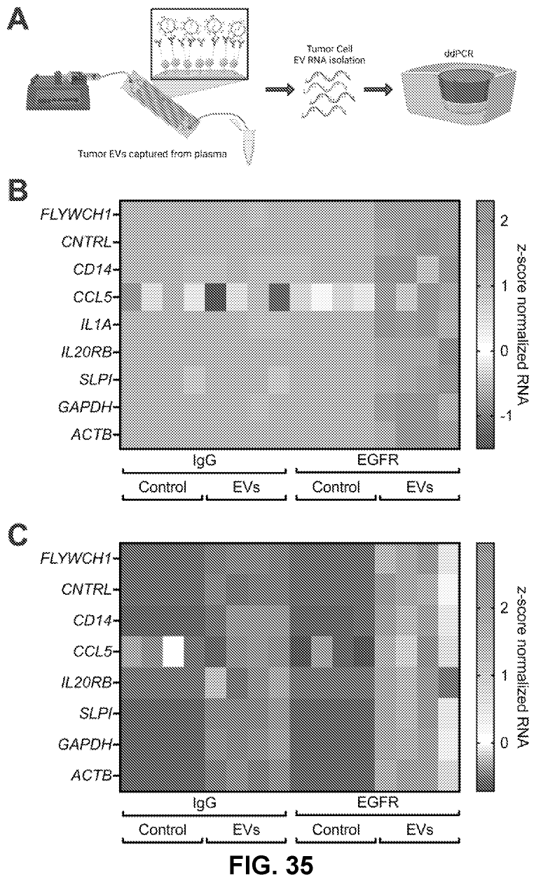

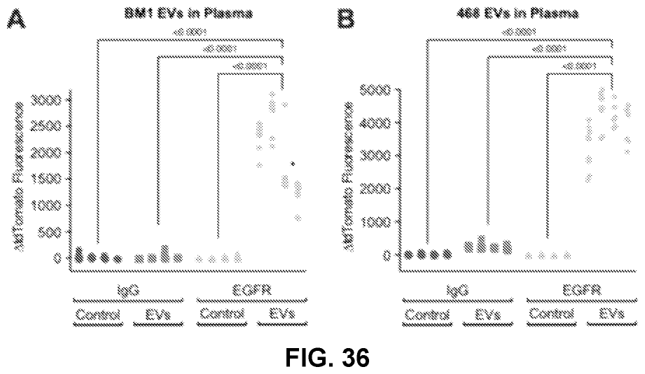

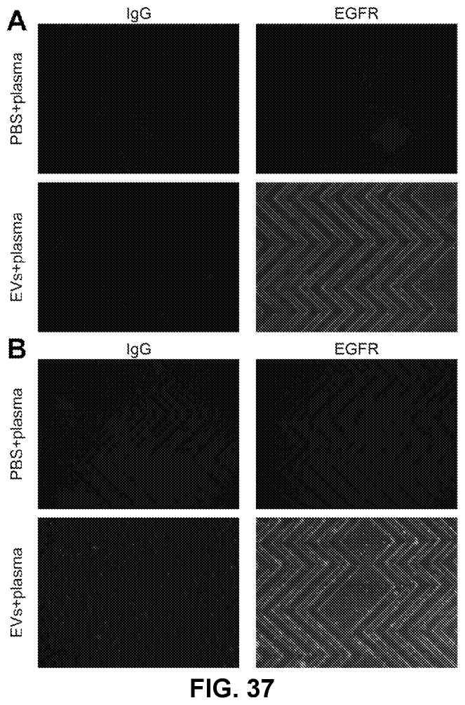

Because of issues with scalability of PDMS devices as well as the higher rates of non-specific capture of EVs on the surface, aryl diazonium functionalized plastic devices were further characterized. Concentrated media containing palmitoylated tdTomato tagged EVs were spiked into normal patient plasma (FIG. 35A). RNA signal from captured tumor cell EVs were compared between plasma alone or plasma spiked with tumor EVs captured on devices with either IgG or anti-EGFR antibody. Only anti-EGFR containing devices captured MDA-MB-231-BM1 or MDA-MB-468 tumor EVs spiked into normal plasma (FIG. 35B-35C, FIG. 36, and FIG. 37). This demonstrates that the functionalization strategy can capture tumor specific EVs from complex biofluids, such as plasma, further demonstrating its potential use in clinical diagnostic assays.

### Example 18: Device Cost and Chemical Safety

While the per device cost is not that high for making PDMS based microfluidic devices, the specialized equipment and time required to create them and functionalize the surface for capture makes them prohibitively expensive at scale. In comparison, microfluidic devices made through injection molding of plastic become cheaper at scale, lowering the per-device cost. Additionally, the procedure for functionalizing devices through aryl diazonium salts requires no expensive equipment purchase by research labs and uses inexpensive chemical reagents (Table 2). This method only requires a low-cost UV bed (frequently available in research labs). Both silane and aryl diazonium wastes require special disposal (Table 3).

When considering the chemical safety of functionalization, silane treatment requires the use of a nitrogen filled glove box to prepare the silane solution, and subsequent steps to functionalize the surface of the devices must be prepared in a chemical fume hood. Additionally, silane functionalization must be performed immediately after oxygen-plasma bonding of PDMS devices. In contrast, use of aryl diazonium only requires a fume hood to weigh powders. The process takes approximately an hour and can be done on the bench. It can be done at a separate time from the bonding procedure, allowing for more flexibility at production. This method works on a variety of surfaces including glass, PDMS, carbon, and plastic. Because it is amenable to a variety of surfaces, it is a convenient method for functionalizing plastic devices. Traditionally, physisorption has been used to add molecules to the surface of devices. However, this process results in a relatively low binding capacity of the device and poor nanoparticle recovery.

TABLE 2
Time, cost, and stability of different methods (all costs in US dollars).

| | Plastic Aryl | Plastic | PDMS-Glass | PDMS-Glass |
|---|---|---|---|---|
| | Diazonium | Physisorption | Aryl Diazonium | Silane |
| Device Cost (chip⁻¹) | $11 | $11 | $30 | $30 |
| Reagent Cost (chip⁻¹) | $5.89 | $11.45 | $5.89 | $1.06 |
| Equipment Cost | $2,500 | $0 | $22,500 | $20,000 |
| Equipment needed | UV light bed | — | UV light bed | ^{#}Clean Room |
| | | | ^{#}Clean Room | Equip. |
| | | | Equip. | |
| Units day⁻¹ | >100,000 | >100,000 | hundreds | hundreds |
| (production) | | | | |
| Time (bonding 10 | — | — | 2.5 hours | 2.5 hours |
| devices) | | | | |
| Time | 2 hours | 2 hours | 2 hours | 2 hours |
| (functionalizing) | | | | |
| Units day⁻¹ | 100-200 | 100-200 | 100-200 | 10-20 |
| (functionalize) | | | | |
| Stability (months) | 6 months, 25° C. | 3 months, 4° C. | Not tested | 6 months, 25° C. |

^{#}Clean Room Equipment includes March Instruments PX-250 plasma asher and Baker BTS-220 SU-8 developer.

TABLE 3
Toxicity of reagents in aryl-diazonium versus silane

| | Toxicity Category | | | | NFPA Ratings | | | |
|---|---|---|---|---|---|---|---|---|
| | Oral | Dermal | Inhalation | Eye | Health | Flammability | Instability | Shipping |
| 3-mercapto | 4 | 3 | 3 | 1 | 3 | 2 | 1 | Class 9 |
| propyl | | | | | | | | |
| trimethoxy | | | | | | | | |
| silane | | | | | | | | |
| p-phenylene | 3 | 3 | 3 | 2 | 3 | 0 | 0 | Group |
| diamine | | | | | | | | III |
| sodium | 3 | 3 | 3 | 3 | 3 | 0 | 2 | Group |
| nitrite | | | | | | | | III |

Overall, the use of PDMS silane-GMBS functionalized microfluidic devices requires high up-front equipment costs as well as access to a clean room for production of silicon wafers and pouring and bonding PDMS devices to glass slides. Because of the specialized equipment and time required for functionalization per device, PDMS devices have limitations when considering scaling of microfluidics for clinical assays. Rapid, mass production of microfluidic devices for clinical assays will require the use of molded plastic devices. To produce an easily translatable method of functionalizing the surface of plastic COC devices, an aryl diazonium salt-based process can be employed, which in turn can include reacting with Biotin-NHS ester or another member of a binding pair to provide the capture agent. This produced a robust, even, stable, and inexpensive method of deposition of biotin across the entire surface of the tested devices. Because these devices are stable for at least three months after functionalization at room temperature, they will be much easier to produce and then distribute to clinical labs for analysis. Additionally, because the instrumentation and chemicals needed for this method are relatively inexpensive and readily available, it will allow research labs to functionalize plastic devices purchased through various vendors. Further, this method has a much higher rate of EV capture from plasma with a device coating compared to other methods of functionalizing plastic devices. This will allow for future analysis of rare cells, vesicles, viruses, or other particles from blood, plasma, or other complex patient biofluids using mass-produced injection molded plastic devices.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed above), scientific articles, patent publications, and any other documents cited herein are hereby incorporated by reference for the substance of their disclosure.

1. A microfluidic device comprising:
a microchannel comprising an inner wall surface;
a first linker covalently attached to the inner wall surface, or a portion thereof;
a particle attached to the first linker; and
a capture agent attached to the particle.

2. The device of claim 1, wherein the first linker comprises an arylene moiety.

3. The device of claim 1, wherein at least one groove is defined in the inner wall surface.

4. The device of claim 1, wherein the first linker comprises —Ar—NR^{N1}—, Ar is optionally substituted arylene, and R^{N1} is hydrogen (H) or C₁₋₆ alkyl.

5. The device of claim 4, wherein Ar is para-phenylene.

6. The device of claim 1, further comprising a first binding pair disposed between first linker and the particle and/or a second binding pair disposed between the particle and the capture agent.

7. The device of claim 1, wherein the capture agent is configured to interact with a surface of a virus in an intact form, a surface of a target cell, or a surface of a target vesicle (e.g., a target extracellular vesicle).

8. The device of claim 1, wherein the capture agent comprises a molecule configured to bind a protein or a nucleic acid (e.g., DNA, RNA, or a modified form thereof).

9. The device of claim 8, wherein the molecule comprises a protein or a nucleic acid.

10. The device of claim 8, wherein the capture agent comprises angiotensin-converting enzyme 2 (ACE2), a mutant form thereof, or a recombinant form thereof.

11. The device of claim 8, wherein the capture agent comprises C—C chemokine receptor type 5 (CCR5), a mutant form thereof, or a recombinant form thereof.

12. The device of claim 8, wherein the capture agent comprises cluster of differentiation 4 (CD4), a mutant form thereof, or a recombinant form thereof.

13. The device of claim 8, wherein the capture agent comprises neutralizing antibody, KZ52, a mutant form thereof, or a recombinant form thereof.

14. The device of claim 8, wherein the capture agent comprises laminin-5, a mutant form thereof, or a recombinant form thereof.

15. The device of claim 8, wherein the capture agent comprises heparin sulfate proteoglycan, a mutant form thereof, or a recombinant form thereof.

16. The device of claim 8, wherein the capture agent comprises cluster of differentiation 46 (CD46), a mutant form thereof, or a recombinant form thereof.

17. The device of claim 8, wherein the capture agent comprises complement receptor type 2 (CR2), a mutant form thereof, or a recombinant form thereof.

18. The device of claim 8, wherein the capture agent comprises an antibody.

19. The device of claim 18, wherein the antibody binds a spike protein of the virus or a receptor binding domain (RBD) of the virus.

20. The device of claim 18, wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR).

21. The device of claim 8, wherein the capture agent comprises an aptamer.

22. The device of claim 21, wherein the aptamer binds a spike protein of the virus.

23. The device of any one of claims 1-22, further comprising:
a second linker disposed between the particle and the capture agent.

24. The device of claim 23, wherein the second linker comprise a flexible linker.

25. The device of claim 24, further comprising a binding pair between the particle and the flexible linker.

26. The device of any one of claims 1-25, wherein the inner wall surface comprises a plurality of grooves arranged and configured to generate chaotic flow within a fluid sample traveling through the microchannel.

27. The device of any one of claims 1-26, wherein the capture agent is configured to capture a virus.

28. The device of claim 27, wherein the virus comprises a cytomegalovirus, a coronavirus, an ebolavirus, an Epstein-Barr virus, a human immunodeficiency virus, an influenza virus, a hepatitis virus, or an oncovirus (e.g., a retrovirus, a herpesvirus, a papillomavirus, a polyomavirus, a hepatitis virus, and the like).

29. A microfluidic system comprising:
a first microchannel comprising a first inner wall surface, wherein at least one groove is defined in the first inner wall surface, and wherein the first microchannel comprises a first capture agent configured to interact with surface of a virus in an intact form.

30. The system of claim 29, wherein the virus comprises a cytomegalovirus, a coronavirus, an ebolavirus, an Epstein-Barr virus, a human immunodeficiency virus, an influenza virus, a hepatitis virus, or an oncovirus (e.g., a retrovirus, a herpesvirus, a papillomavirus, a polyomavirus, a hepatitis virus, and the like).

31. The system of claim 29, further comprising:
a second microchannel comprising a second inner wall surface, wherein at least one groove is defined in the second inner wall surface, and wherein the second microchannel comprises a second capture agent configured to interact with a surface of a target cell or a surface of a target vesicle in an intact form; and
a fluidic interconnect configured to provide fluidic communication between an outlet of the first microchannel to an inlet of the second microchannel.

32. The system of claim 31, wherein the target cell comprises an immune cell (e.g., a T cell, a B cell, or an innate immune cell), an epithelial cell, an endothelial cell, or a neural cell; or wherein the target vesicle comprises an extracellular vesicle, a vesicle from an immune cell (e.g., a T cell, a B cell, or an innate immune cell), a vesicle from an epithelial cell, a vesicle from an endothelial cell, a vesicle from a neural cell, or a vesicle from a damaged cell.

33. The system of any one of claims 29-32, wherein the first microchannel is provided as the microchannel in the microfluidic device of any one of claims 1-28.

34. The system of any one of claims 31-33, wherein the second microchannel is provided as the microchannel in the microfluidic device of any one of claims 1-28.

35. The system of claim 34, wherein the first capture agent and the second capture agent are different.

36. A microfluidic system comprising:
a first microchannel comprising a first inner wall surface, wherein at least one groove is defined in the first inner wall surface, and wherein the first microchannel comprises a first capture agent configured to interact with a surface of a virus in an intact form;
a second microchannel comprising a second inner wall surface, wherein at least one groove is defined in the second inner wall surface, and wherein the second microchannel comprises a second capture agent configured to interact with a surface of a target cell or a surface of a target vesicle in an intact form; and
a first fluidic interconnect configured to provide fluidic communication between an outlet of the first microchannel to an inlet of the second microchannel,
wherein:
the target cell comprises a B cell, an innate immune cell (e.g., a neutrophil, a macrophage, etc.), an epithelial cell, an endothelial cell, or a neural cell; or
the target vesicle comprises a vesicle from a B cell, a vesicle from an innate immune cell, a vesicle from an epithelial cell, a vesicle from an endothelial cell, a vesicle from a neural cell, or a vesicle from a damaged cell.

37. The system of claim 36, further comprising:
a third microchannel comprising a third inner wall surface, wherein at least one groove is defined in the third inner wall surface, and wherein the third microchannel comprises a third capture agent configured to interact with a surface of a target cell or a surface of a target vesicle in an intact form; and
a second fluidic interconnect configured to provide fluidic communication between an outlet of the second microchannel to an inlet of the third microchannel.

38. The system of claim 37, wherein the second capture agent and the third capture agent are different.

39. The system of any one of claims 36-38, wherein the first microchannel is provided as the microchannel in the microfluidic device of any one of claims 1-28.

40. The system of any one of claims 36-39, wherein the second microchannel is provided as the microchannel in the microfluidic device of any one of claims 1-28.

41. The system of any one of claims 36-40, wherein the third microchannel is provided as the microchannel in the microfluidic device of any one of claims 1-28.

42. A method of isolating a virus in a sample, the method comprising:
flowing the sample comprising the virus through a first microchannel comprising a first inner wall surface, wherein at least one groove is defined in the first inner wall surface, and wherein the first inner wall surface comprises a first capture agent configured to interact with a surface of the virus;
capturing the virus in an intact form using the first capture agent in the first microchannel;
lysing the intact form of the virus in the first microchannel, thereby providing a lysate; and
analyzing the lysate to determine the presence of one or more markers of the virus.

43. The method of claim 42, wherein said capturing comprises contacting the virus in the intact form with the first capture agent.

44. The method of claim 42 or 43, wherein the first capture agent comprises a protein.

45. The method of claim 44, wherein the protein comprises angiotensin-converting enzyme 2 (ACE2), a mutant form thereof, or a recombinant form thereof.

46. The method of claim 44, wherein the protein comprises an antibody.

47. The method of claim 46, wherein the antibody binds a spike protein of the virus or a receptor binding domain (RBD) of the virus.

48. The method of claim 42 or 43, wherein the first capture agent comprises an aptamer.

49. The method of claim 49, wherein the aptamer binds a spike protein of the virus.

50. The method of claim 42, wherein said capturing comprises capturing at least one intact form of the virus in a microliter of the sample.

51. The method of any one of claims 42-50, wherein said lysing comprises exposing the first microchannel to an elevated temperature, a lysing agent, or both.

52. The method of claim 42, wherein the virus comprises a coronavirus, an ebolavirus, an influenza virus, a hepatitis virus, or an oncovirus (e.g., a retrovirus, a herpesvirus, a papillomavirus, a polyomavirus, a hepatitis virus, and the like).

53. The method of any one of claims 42-52, wherein the virus in the intact form is a viral particle.

54. The method of any one of claims 42-53, wherein said analyzing comprises amplifying or sequencing the one or more markers.

55. The method of claim 54, wherein said amplifying comprises conducting an isothermal amplification reaction.

56. The method of any one of claims 42-55, wherein the one or more markers comprises a nucleic acid.

57. The method of any one of claims 42-56, wherein the sample comprises a diluted sample, a stabilized sample, a preserved sample, or a combination thereof.

58. The method of any one of claims 42-57, wherein the sample comprises blood, plasma, stool, saliva, urine, sputum, or waste water.

59. The method of any one of claims 42-58, wherein said flowing comprises flowing the sample through the microchannel of the microfluidic device of any one of claims 1-28 or through the first microchannel of the microfluidic system of any one of claims 29-41.

60. The method of any one of claims 42-59, further comprising, prior to said flowing the sample:
diluting the sample with a diluent to provide a diluted sample, wherein the diluted sample is used as the sample during said flowing of the sample through the first microchannel.

61. The method of any one of claims 42-60, further comprising, prior to said flowing the sample:
stabilizing the sample with a stabilizer to provide a stabilized sample (e.g., by use of particular storage temperatures, platelet inhibitor cocktail, chemical additive, and the like), wherein the stabilized sample is used as the sample during said flowing of the sample through the first microchannel.

62. The method of any one of claims 42-61, further comprising, prior to said lysing the intact form of the virus:
determining a concentration of the virus captured by the first capture agent in the first microchannel.

63. The method of claim 62, wherein said determining comprises conducting one or more optical measurements, amplification reactions, sequencing, resistive pulse sensing, or particle analysis to measure a concentration of viral particles captured by the first capture agent in the first microchannel.

64. The method of any one of claims 42-63, further comprising, after said lysing the intact form of the virus:
delivering one or more detection reagents to the first microchannel.

65. The method of claim 64, wherein the one or more detection reagents are employed during said analyzing the lysate to conduct an isothermal amplification reaction within the first microchannel.

66. The method of any one of claims 42-65, further comprising, after said flowing the sample comprising virus though the first microchannel:
collecting the sample after flowing through the first microchannel, thereby providing a collected sample comprising one or more target cells or target vesicles; and
flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle.

67. The method of claim 66, wherein the second capture agent comprises a protein.

68. The method of claim 67, wherein the protein comprises an antibody, and optionally wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR).

69. The method of any one of claims 66-68, wherein the target cell comprises an immune cell (e.g., a T cell or a B cell), an epithelial cell, an endothelial cell, or a neural cell; or wherein the target vesicle comprises an extracellular vesicle, a vesicle from an immune cell (e.g., a T cell or a B cell), a vesicle from an epithelial cell, a vesicle from an endothelial cell, a vesicle from a neural cell, or a vesicle from a damaged cell.

70. A method of capturing targets in a sample, the method comprising:
flowing the sample comprising a virus through a first microchannel comprising a first inner wall surface, wherein at least one groove is defined in the first inner wall surface, and wherein the first inner wall surface comprises a first capture agent configured to interact with a surface of the virus;
capturing the virus in an intact form using the first capture agent in the first microchannel;
collecting the sample after flowing through the first microchannel, thereby providing a collected sample comprising one or more target cells or target vesicles;
flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle; and
capturing the target cell or the target vesicle in an intact form using the second capture agent in the second microchannel,
wherein:
the target cell comprises a B cell, an innate immune cell (e.g., a neutrophil, a macrophage, etc.), an epithelial cell, an endothelial cell, or a neural cell; or
the target vesicle comprises a vesicle from a B cell, a vesicle from an innate immune cell, a vesicle from an epithelial cell, a vesicle from an endothelial cell, a vesicle from a neural cell, or a vesicle from a damaged cell.

71. The method of claim 70, further comprising:
lysing the intact form of the virus in the first microchannel, thereby providing a first lysate; and
analyzing the first lysate to determine the presence of one or more markers of the virus.

72. The method of claim 70 or 71, further comprising:
lysing the target cell or the target vesicle in the second microchannel, thereby providing a second lysate; and
analyzing the second lysate to determine the presence of one or more markers of the target cell or the target vesicle.

73. The method of claim 71 or 72, wherein said lysing comprises exposing the first microchannel to an elevated temperature, a lysing agent, or both.

74. The method of claim 70 or 71, wherein the first capture agent comprises a protein.

75. The method of claim 74, wherein the protein comprises angiotensin-converting enzyme 2 (ACE2), a mutant form thereof, or a recombinant form thereof.

76. The method of claim 74, wherein the protein comprises an antibody.

77. The method of claim 76, wherein the antibody binds a spike protein of the virus or a receptor binding domain (RBD) of the virus.

78. The method of claim 71 or 72, wherein the first capture agent comprises an aptamer.

79. The method of claim 78, wherein the aptamer binds a spike protein of the virus.

80. The method of claim 70, wherein said capturing comprises capturing at least one intact form of the virus in a microliter of the sample.

81. The method of any one of claims 71-80, wherein said lysing comprises exposing the first microchannel and/or the second microchannel to an elevated temperature, a lysing agent, or both.

82. The method of claim 70, wherein the virus comprises a coronavirus, an ebolavirus, an influenza virus, a hepatitis virus, or an oncovirus (e.g., a retrovirus, a herpesvirus, a papillomavirus, a polyomavirus, a hepatitis virus, and the like).

83. The method of any one of claims 70-82, wherein the virus in the intact form is a viral particle.

84. The method of any one of claims 71-83, wherein said analyzing comprises amplifying or sequencing the one or more markers.

85. The method of claim 84, wherein said amplifying comprises conducting an isothermal amplification reaction.

86. The method of any one of claims 71-85, wherein the one or more markers comprises a nucleic acid.

87. The method of any one of claims 70-86, wherein the sample comprises a diluted sample, a stabilized sample, a preserved sample, or a combination thereof.

88. The method of any one of claims 70-87, wherein the sample comprises blood, plasma, stool, saliva, urine, sputum, or waste water.

89. The method of any one of claims 70-88, wherein said flowing comprises flowing the sample through the microchannel of the microfluidic device of any one of claims 1-28 or through the first microchannel of the microfluidic system of any one of claims 29-41.

90. The method of any one of claims 70-89, further comprising, prior to said flowing the sample:
diluting the sample with a diluent to provide a diluted sample, wherein the diluted sample is used as the sample during said flowing of the sample through the first microchannel.

91. The method of any one of claims 70-90, further comprising, prior to said flowing the sample:
stabilizing the sample with a stabilizer to provide a stabilized sample (e.g., by use of particular storage temperatures, platelet inhibitor cocktail, chemical additive, and the like), wherein the stabilized sample is used as the sample during said flowing of the sample through the first microchannel.

92. The method of any one of claims 71-91, further comprising, prior to said lysing the intact form of the virus:
determining a concentration of the virus captured by the first capture agent in the first microchannel.

93. The method of claim 92, wherein said determining comprises conducting one or more optical measurements, amplification reactions, sequencing, resistive pulse sensing, or particle analysis to measure a concentration of viral particles captured by the first capture agent in the first microchannel.

94. The method of any one of claims 71-94, further comprising, after said lysing the intact form of the virus:
delivering one or more detection reagents to the first microchannel.

95. The method of claim 94, wherein the one or more detection reagents are employed during said analyzing the lysate to conduct an isothermal amplification reaction within the first microchannel.

96. The method of any one of claims 70-95, further comprising, after said flowing the sample comprising virus though the first microchannel:
collecting the sample after flowing through the first microchannel, thereby providing a collected sample comprising one or more target cells or target vesicles; and
flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle.

97. The method of claim 96, wherein the second capture agent comprises a protein.

98. The method of claim 97, wherein the protein comprises an antibody, and optionally wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR).

99. The method of any one of claims 70-98, wherein the target cell comprises an immune cell (e.g., a T cell or a B cell), an epithelial cell, an endothelial cell, or a neural cell; or wherein the target vesicle comprises an extracellular vesicle, a vesicle from an immune cell (e.g., a T cell or a B cell), a vesicle from an epithelial cell, a vesicle from an endothelial cell, a vesicle from a neural cell, or a vesicle from a damaged cell.

100. A method of determining viral load in a sample, the method comprising:
flowing the sample through a first microchannel comprising a first inner wall surface, wherein at least one groove is defined in the first inner wall surface, and wherein the first inner wall surface comprises a first capture agent configured to interact with a surface of a viral particle;
capturing the viral particle in an intact form using the first capture agent in the first microchannel; and
measuring a concentration of viral particle captured in the first microchannel.

101. The method of claim 100, wherein said measuring comprises conducting one or more optical measurements, amplification reactions, sequencing, resistive pulse sensing, or particle analysis to measure a concentration of viral particles captured by the first capture agent in the first microchannel.

102. The method of claim 100, further comprising (e.g., after said capturing):
lysing the viral particle in the first microchannel, thereby providing a lysate; and
analyzing the lysate to determine the presence of one or more markers of the viral particle.

103. The method of any one of claims 100-102, further comprising (e.g., after said capturing):
collecting the sample after flowing through the first microchannel, thereby providing a collected sample comprising one or more target cells or target vesicles;
flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle; and
capturing the target cell or the target vesicle in an intact form using the second capture agent in the second microchannel.

104. The method of claim 103, further comprising:
lysing the target cell or the target vesicle in the second microchannel, thereby providing a second lysate; and
analyzing the second lysate to determine the presence of one or more markers of the target cell or the target vesicle.

105. The method of any one of claims 100-104, wherein the first capture agent comprises a protein.

106. The method of claim 105, wherein the protein comprises angiotensin-converting enzyme 2 (ACE2), a mutant form thereof, or a recombinant form thereof.

107. The method of claim 105, wherein the protein comprises an antibody.

108. The method of claim 107, wherein the antibody binds a spike protein of the virus or a receptor binding domain (RBD) of the virus.

109. The method of any one of claims 100-104, wherein the first capture agent comprises an aptamer.

110. The method of claim 109 wherein the aptamer binds a spike protein of the virus.

111. The method of claim 100, wherein said capturing comprises capturing at least one intact form of the virus in a microliter of the sample.

112. The method of any one of claims 102-111, wherein said lysing comprises exposing the first microchannel to an elevated temperature, a lysing agent, or both.

113. The method of claim 100, wherein the virus comprises a coronavirus, an ebolavirus, an influenza virus, a hepatitis virus, or an oncovirus (e.g., a retrovirus, a herpesvirus, a papillomavirus, a polyomavirus, a hepatitis virus, and the like).

114. The method of any one of claims 100-113, wherein the virus in the intact form is a viral particle.

115. The method of any one of claims 102-114, wherein said analyzing comprises amplifying or sequencing the one or more markers.

116. The method of claim 115, wherein said amplifying comprises conducting an isothermal amplification reaction.

117. The method of any one of claims 100-116, wherein the one or more markers comprises a nucleic acid.

118. The method of any one of claims 100-117, wherein the sample comprises a diluted sample, a stabilized sample, a preserved sample, or a combination thereof.

119. The method of any one of claims 100-118, wherein the sample comprises blood, plasma, stool, saliva, urine, sputum, or waste water.

120. The method of any one of claims 100-119, wherein said flowing comprises flowing the sample through the microchannel of the microfluidic device of any one of claims 1-28 or through the first microchannel of the microfluidic system of any one of claims 29-41.

121. The method of any one of claims 100-120, further comprising, prior to said flowing the sample:
diluting the sample with a diluent to provide a diluted sample, wherein the diluted sample is used as the sample during said flowing of the sample through the first microchannel.

122. The method of any one of claims 100-122, further comprising, prior to said flowing the sample:
stabilizing the sample with a stabilizer to provide a stabilized sample (e.g., by use of particular storage temperatures, platelet inhibitor cocktail, chemical additive, and the like), wherein the stabilized sample is used as the sample during said flowing of the sample through the first microchannel.

123. The method of any one of claims 102-122, further comprising, prior to said lysing the intact form of the virus:
determining a concentration of the virus captured by the first capture agent in the first microchannel.

124. The method of claim 123, wherein said determining comprises conducting one or more optical measurements, amplification reactions, sequencing, resistive pulse sensing, or particle analysis to measure a concentration of viral particles captured by the first capture agent in the first microchannel.

125. The method of any one of claims 102-124, further comprising, after said lysing the intact form of the virus:
delivering one or more detection reagents to the first microchannel.

126. The method of claim 128, wherein the one or more detection reagents are employed during said analyzing the lysate to conduct an isothermal amplification reaction within the first microchannel.

127. The method of any one of claims 100-127, further comprising, after said flowing the sample comprising virus though the first microchannel:
collecting the sample after flowing through the first microchannel, thereby providing a collected sample comprising one or more target cells or target vesicles; and
flowing the collected sample through a second microchannel comprising a second inner wall surface, wherein at least one groove is defined in the second inner wall surface, and wherein the second inner wall surface comprises a second capture agent configured to interact with a surface of the target cell or a surface of the target vesicle.

128. The method of claim 127, wherein the second capture agent comprises a protein.

129. The method of claim 128, wherein the protein comprises an antibody, and optionally wherein the antibody binds to CD3, CD4, CD8, CD9, CD11b, CD19, CD20, CD63, CD66b, CD81, HLA-DR, TSG-101, epithelial cell adhesion molecule (EpCAM), or epidermal growth factor receptor (EGFR).

130. The method of any one of claims 103-129, wherein the target cell comprises an immune cell (e.g., a T cell or a B cell), an epithelial cell, an endothelial cell, or a neural cell; or wherein the target vesicle comprises an extracellular vesicle, a vesicle from an immune cell (e.g., a T cell or a B cell), a vesicle from an epithelial cell, a vesicle from an endothelial cell, a vesicle from a neural cell, or a vesicle from a damaged cell.

131. A method of preparing a microfluidic device, the method comprising:
forming an aryl-onium salt comprising an arylene moiety disposed between an onium group and a nucleophilic group;
releasing the onium group to form an aryl radical; and
exposing the aryl radical to a surface of a microfluidic device, thereby providing a functionalized surface.

132. The method of claim 131, further comprising, before said releasing:
reacting the nucleophilic group of the aryl-onium salt with a first member of a first binding pair to form an aryl conjugate, wherein the aryl conjugate comprises the arylene moiety disposed between the onium group and the first member of the binding pair, and wherein the aryl conjugate is employed during said releasing to provide the aryl radical.

133. The method of claim 131, further comprising, after said exposing:
reacting the nucleophilic group of the functionalized surface with a first member of a first binding pair.

134. The method of claim 132 or 133, further comprising:
providing a particle comprising a second member of the first binding pair, wherein the first and second members bind together to form a bond.

135. The method of claim 134, further comprising:
attaching one or more capture agents to the particle.

136. The method of claim 135, wherein a linker is disposed between at least one of the one or more capture agents and the particle.

137. The method of claim 136, wherein the linker comprises a flexible linker.

138. The method of claim 137, wherein a second binding pair is disposed between the particle and the flexible linker, wherein the second member of the first binding pair of the particle is employed as a first member of the second binding pair, and wherein a second member of the second binding pair is attached to the flexible linker.

139. The method of claim 132 or 133, further comprising:
providing a linker comprising a second member of the first binding pair, wherein the first and second members bind together to form a bond.

140. The method of claim 139, further comprising:
attaching one or more capture agents to the linker.

141. The method of claim 140, wherein the linker comprises a flexible linker.

142. The method of claim 141, wherein a second binding pair is disposed between the flexible linker and at least one of the one or more capture agents.

143. The method of any one of claims 131-142, wherein said forming comprises exposing an arylene compound to an oxidant (e.g., nitrous acid or a nitrite salt) and an optional acid (e.g., hydrogen halide).

144. The method of any one of claims 131-143, wherein the onium group and the nucleophilic group are in a para position.

145. The method of any one of claims 131-144, wherein the arylene moiety comprises phenylene; or wherein the onium group comprises diazonium, iodonium, bromonium, or sulfonium; or wherein the nucleophilic group comprises amino (e.g., —NR^{N1}R^{N2}, wherein each of R^{N1} and R^{N2} is, independently, hydrogen or C₁₋₆ alkyl).

146. The method of any one of claims 131-145, wherein a terminal amino group (e.g., of the aryl-onium salt, the arylene moiety, the nucleophilic group, the aryl radical, the aryl conjugate, the particle, or the linker) is reacted prior to addition to the microfluidic device or exposure to the surface of the microfluidic device.

147. The method of any one of claims 131-146, wherein said releasing comprises exposing the onium group to radiation (e.g., ultraviolet radiation), heat, or electric field.

148. The method of any one of claims 131-147, wherein said releasing the onium group occurs in the presence of the surface of the microfluidic device.

149. The method of claim 148, wherein the surface of the microfluidic device comprises an inner wall surface of a microchannel.

150. The method of claim 149, wherein at least one groove is defined in the inner wall surface.

151. The method of claim 150, wherein the inner wall surface comprises a plurality of grooves arranged and configured to generate chaotic flow within a fluid sample traveling through the microchannel.


SEQUENCE LISTING (30 sequences)

SEQ ID NO: 1 - DNA, 17 bp, synthetic construct
  source 1..17: mol_type = other DNA
ccttgcacatgccggag

SEQ ID NO: 2 - DNA, 17 bp, synthetic construct
  source 1..17: mol_type = other DNA
acagagcctcgcctttg

SEQ ID NO: 3 - DNA, 21 bp, synthetic construct
  source 1..21: mol_type = other DNA
tcatccatggtgagctggcgg

SEQ ID NO: 4 - DNA, 22 bp, synthetic construct
  source 1..22: mol_type = other DNA
gactctccatcctagctcatct

SEQ ID NO: 5 - DNA, 22 bp, synthetic construct
  source 1..22: mol_type = other DNA
gagtatttctacaccagtggca

SEQ ID NO: 6 - DNA, 28 bp, synthetic construct
  source 1..28: mol_type = other DNA
atgtactcccgaacccatttcttctctg

SEQ ID NO: 7 - DNA, 21 bp, synthetic construct
  source 1..21: mol_type = other DNA
aatcttcatcgtccagctcac

SEQ ID NO: 8 - DNA, 22 bp, synthetic construct
  source 1..22: mol_type = other DNA
cagaggttcggaagacttatcg

SEQ ID NO: 9 - DNA, 19 bp, synthetic construct
  source 1..19: mol_type = other DNA
cgcagagacgtgcaccagc

SEQ ID NO: 10 - DNA, 22 bp, synthetic construct
  source 1..22: mol_type = other DNA
cattttccacctccgttcattg

SEQ ID NO: 11 - DNA, 24 bp, synthetic construct
  source 1..24: mol_type = other DNA
gtctctttccagtctttctacctc

SEQ ID NO: 12 - DNA, 25 bp, synthetic construct
  source 1..25: mol_type = other DNA
ttggaaggtcagccagtaaccactc

SEQ ID NO: 13 - DNA, 20 bp, synthetic construct
  source 1..20: mol_type = other DNA
tcttcatcttgggcagtcac

SEQ ID NO: 14 - DNA, 22 bp, synthetic construct
  source 1..22: mol_type = other DNA
gctgctgcattacataatctgg

SEQ ID NO: 15 - DNA, 27 bp, synthetic construct
  source 1..27: mol_type = other DNA
tgaagcagtgaaatttgacatgggtgc

SEQ ID NO: 16 - DNA, 20 bp, synthetic construct
  source 1..20: mol_type = other DNA
gaccttcagtgagtgagcac

SEQ ID NO: 17 - DNA, 22 bp, synthetic construct
  source 1..22: mol_type = other DNA
accaacatgaagcatctcttga

SEQ ID NO: 18 - DNA, 23 bp, synthetic construct
  source 1..23: mol_type = other DNA
agcctgtacacgagccacatctg

SEQ ID NO: 19 - DNA, 22 bp, synthetic construct
  source 1..22: mol_type = other DNA
tgtagttgaggtcaatgaaggg

SEQ ID NO: 20 - DNA, 19 bp, synthetic construct
  source 1..19: mol_type = other DNA
acatcgctcagacaccatg

SEQ ID NO: 21 - DNA, 25 bp, synthetic construct
  source 1..25: mol_type = other DNA
aaggtcggagtcaacggatttggtc

SEQ ID NO: 22 - DNA, 19 bp, synthetic construct
  source 1..19: mol_type = other DNA
tgtggaaggctctggaaag

SEQ ID NO: 23 - DNA, 22 bp, synthetic construct
  source 1..22: mol_type = other DNA
tggcactcaggtttcttgtatc

SEQ ID NO: 24 - DNA, 27 bp, synthetic construct
  source 1..27: mol_type = other DNA
tgggcagatttcttaggaggacagact

SEQ ID NO: 25 - DNA, 18 bp, synthetic construct
  source 1..18: mol_type = other DNA
ccagccagccctagagat

SEQ ID NO: 26 - DNA, 19 bp, synthetic construct
  source 1..19: mol_type = other DNA
cactgccttctcctgcttg

SEQ ID NO: 27 - DNA, 22 bp, synthetic construct
  source 1..22: mol_type = other DNA
aggaaggactccagcaccagga

SEQ ID NO: 28-29 - skipped (2 entries)

SEQ ID NO: 30 - protein, 597 aa, synthetic construct
STIEEQAKTFLDFFDSQAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCLPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD